(19) United States
(12) Patent Application Publication
(10) Pub. No.: US 2011/0052600 A1
(43) Pub. Date: Mar. 3, 2011

(54) IL-17RA-IL-17RB ANTAGONISTS AND USES THEREOF

(75) Inventors: Alison L. Budelsky, Seattle, WA (US); Michael R. Comeau, Bainbridge Island, WA (US); Joel E. Tocker, Issaquah, WA (US)

(73) Assignee: AMGEN INC., Thousand Oaks, CA (US)

(21) Appl. No.: 12/918,449

(22) PCT Filed: Feb. 20, 2009
(86) PCT No.: PCT/US09/01085

§ 371 Date: Nov. 15, 2010

Related U.S. Application Data
(60) Provisional application No. 61/066,538, filed on Feb. 21, 2008.
(60) Provisional application No. 61/145,901, filed on Jan. 20, 2009.

(51) Int. Cl. A61K 39/395; C12N 5/071; A61P 37/06

(52) U.S. Cl. 424/158.1; 435/325

(57) ABSTRACT

The present invention relates to Interleukin-17 ligand and receptor family members and the discovery that IL-17 receptor A and IL-17 receptor C form a heteromeric receptor complex that is biologically active. Antagonists of the IL-17RA-IL-17RB heteromeric receptor complex are disclosed, as well as various methods of use.

### CROSS REFERENCE TO RELATED APPLICATIONS

This application claims the benefit under 35 U.S.C. §119 of U.S. Provisional Application Ser. No. 61/145, 901, filed Jan. 20, 2009 and U.S. Provisional Application Ser. No. 61/066,538, filed Feb. 212, 2008, which are hereby incorporated by reference.

### FIELD OF THE INVENTION

The present invention relates to Interleukin-17 ligand and receptor family members and the discovery that IL-17 receptor A and IL-17 receptor B form a heteromeric complex that is biologically active. Antagonists of the IL-17RA-IL-17RB heteromeric receptor complex and methods of use are disclosed.

### BACKGROUND OF THE INVENTION

The Interleukin-17 family is a group of six structurally related cytokines, designated IL-17A through IL-17F, that are important in the regulation of immune responses. At the primary structure level, the greatest similarity appears to be in the C-terminal region, which contains four conserved cysteine residues (reviewed in Kawaguchi et al., J. Allergy Clin Immunol 114:1265, 2004; Kolls and Linden, Immunity 21:467, 2004). The crystal structure of IL-17F has been determined, and found to share structural features with cystine knot family growth factors (Hymowitz, et al., 2001, EMBO J. 20:5532-5341), a group of homodimeric ligands that bind and signal through both homodimeric and heteromeric counterstructures (Lu, et al., 2005, Nat. Rev. Neurosci. 6: 603-614; Barker, 2004, Neuron 42:529-533).

IL-17 receptors (IL-17R) also form a family of related Type I transmembrane proteins. Five different members of this family have been identified (IL-1RA through IL-1RE), several of which also display alternative splicing including soluble forms that may act as decoy receptors (Kolls and Linden, supra; Moseley et al., Cytokine Growth Factor Rev. 14:155, 2003). Although IL-17RA can multimerize, independent of ligand, and has been shown to form a biologically active heteromeric receptor complex with IL-17RC (Toy et al., J Immunol. 177:36; 2007), the possibility of formation of other heteromeric IL-17R complexes (either transmembrane or soluble forms), and resulting biological activity, if any, was previously unknown. This, and other aspects of the various embodiments of the invention, are provided.

### BRIEF DESCRIPTION OF THE FIGURES

FIG. 1 is a graph illustrating the effects on airway hyperresponsiveness of an IL-17RA-IL-17RB antagonist. The open circles depict results obtained in mice (N=4) given MSA and MuFc; the open squares depict results obtained in mice (N=3) given IL-25 and MuFc; the closed triangles depict results obtained in mice (N=3) given IL-25 and M751.

FIG. 2 presents a Western blot, prepared substantially as described in Example 11. Lanes 1 and 4 contain molecular weight markers. Panel A was blotted with anti-IL-17RA, Panel B was blotted with anti-HIS. Lane 2 presents an IL-17RA:HIS positive control, lane 3 shows the results of precipitating IL-17RA:HIS with IL-17RB:Fc. Lane 5 presents an IL-17RD:HIS positive control, lane 6 indicates that IL-17RD:HIS cannot be precipitated with IL-17RB:Fc.

FIG. 3 is a graph illustrating airway hyperresponsiveness (AHR) of mice in an OVA asthma model from Example 14, Experiment 1. Mice were challenged with increasing concentrations of methacholine and the change in PENH above baseline±SEM was calculated.

FIG. 4 illustrates pulmonary resistance (RL) in mice in an OVA asthma model as described in Example 14. Mean airway resistance (R) area under the curve (AUC) is shown for each treatment group±SEM. FIG. 4a presents results from Experiment 2, 4b from Experiment 3.

FIG. 5 presents the analysis of the bronchoalveolar lavage fluid (BALF) cellularity as described in Example 14, experiment 1. Results shown are total BALF (4a), leukocytes, (4b) eosinophils, (4c) neutrophils, (4d) lymphocytes, and (4e) macrophages. Each closed circle represents BALF cellularity from one mouse. Statistical analyses comparisons were performed using a nonparametric one way ANOVA with Dunn's Multiple Comparison Test (*p<0.05).

FIG. 6 is similar to FIG. 5, but presents results from Experiment 2 of Example 14. Results shown are total BALF (4a), leukocytes, (4b) eosinophils, (4c) neutrophils, (4d) lymphocytes, and (4e) macrophages. Statistical analyses comparisons were performed using a one way ANOVA with Bonferroni's Multiple Comparison Test (*p<0.05).

FIG. 7 presents results from Experiment 3, Example 14. Results shown are total BALF (4a), leukocytes, (4b) eosinophils, (4c) neutrophils, (4d) lymphocytes, and (4e) macrophages. Each closed circle represents BALF cellularity from one mouse. Statistical analyses comparisons were performed using a nonparametric one way ANOVA with Dunn's Multiple Comparison Test (*p<0.05).

FIG. 8 illustrates BALF IL-13 concentrations from mice in an OVA asthma model. IL-13 concentrations were measured by ELISA in BALF samples from individual mice in 3 separate experiments as described in Example 14: (a) Experiment 1, (b) Experiment 2, (c) Experiment 3. Each closed circle indicates a value from one mouse. Horizontal lines indicate group means. Comparisons among groups were performed using a one-way ANOVA. *p<0.05.

FIG. 9 presents BALF IL-5 concentrations from mice in an OVA asthma model. IL-5 concentrations were measured by ELISA in BALF samples from individual mice in 3 separate experiments as described in Example 14: (a) Experiment 1, (b) Experiment 2, (c) Experiment 3. Each closed circle indicates a value from one mouse. Horizontal lines indicate group means. Comparisons among groups were performed using a one-way ANOVA. *p<0.05.

FIG. 10 illustrates serum concentrations of IgE determined by ELISA in individual mice in an OVA asthma model as described in Example 14, in (a) Experiment 1 (b) Experiment 2 (c) Experiment 3. The serum IgE concentration for each mouse is indicated with a closed circle. Horizontal lines indicate group means. Comparisons among groups were performed using a one-way ANOVA. *p<0.05.

FIG. 11 presents lung histology scores from groups of mice in an OVA asthma model as described in Example 14, Experiment 3. *p<0.0001 using an unpaired t-test for statistical comparisons.

### DETAILED DESCRIPTION OF THE INVENTION

The section headings used herein are for organizational purposes only and are not to be construed as limiting the subject matter described.

Standard techniques may be used for recombinant DNA, oligonucleotide synthesis, tissue culture and transformation, protein purification etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manual, 3^{rd} ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytical chemistry, organic chemistry, and medicinal and pharmaceutical chemistry described herein are those well known and commonly used in the art. Standard techniques may be used for chemical synthesis, chemical analyses, pharmaceutical preparation, formulation, and delivery and treatment of patients.

The characterization, cloning, and preparation of IL-17RA are described for example in U.S. Pat. No. 6,072,033, issued Jun. 6, 2000, which is incorporated herein by reference in its entirety. The amino acid sequence of the human IL-17RA is shown in SEQ ID NO:10 of U.S. Pat. No. 6,072,033 (GenBank accession number NM_{—}014339). The human IL-17RA has an N-terminal signal peptide with a predicted cleavage site approximately between amino acid 27 and 28. The signal peptide is followed by a 293 amino acid extracellular domain, a 21 amino acid transmembrane domain, and a 525 amino acid cytoplasmic tail. Soluble forms of human IL-17RA (hulL-17RA) that are useful in the methods of the present invention include the extracellular domain (residues 1-320 or residues 28-320 which excludes the signal peptide) or a fragment of the extracellular domain that retains the capacity to bind IL-17A. Other forms of IL-17RA that are useful in the present invention include muteins and variations that are at least between 70% and 99% amino acid identity to the native IL-17RA that retains the capacity to bind IL-17A, as describe in greater detail in U.S. Pat. No. 6,072,033.

IL-17 Receptor B (IL-17RB) and its many isoforms are known in the art, such as those disclosed and described in Tian et al., Oncogene 19:2098 (2000). Further examples include sequences available on public databases, such as, but not limited to GenBank accession no. NM_{—}018725. In addition, as described below, IL-17RB may also include biologically active fragments and/or variants.

IL-17RA associates with IL-17RB to form a heteromeric receptor complex that is biologically active (i.e. upon binding of ligand, the receptor complex is activated and transduces a signal into a cell upon which it is expressed, resulting in a biological activity such as induction of mRNAs, secretion of cytokines, change in morphology or activation state of the cell, etc.). Each member of a heteromeric receptor complex is referred to as a “component” or “subunit” thereof. As used herein, “IL-17RA-IL-17RB heteromeric receptor complex” (or “heteromeric receptor complex”) refers to a complex comprising at least IL-17RA and IL-17RB; additional subunits or components may also form part of the heteromeric receptor complex.

Thus, certain aspects of the invention are drawn to agents (e.g., antigen binding proteins, as described below) and methods for blocking the association of IL-17RA with IL-17RB (and/or with additional receptor subunits) and thereby preventing formation of a functional receptor complex (a receptor complex that is capable of being activated). Other aspects of the invention are drawn to an antagonist that binds the IL-17RA-IL-17RB heteromeric receptor complex, or a subunit or component thereof, and inhibits binding of ligand (i.e., IL-25) and subsequent activation of the receptor complex. Still further aspects of the invention are drawn to antagonists that bind an IL-17RA-IL-17RB heteromeric receptor complex or a subunit thereof, and prevent activation from occurring. Preventing a functional receptor complex from being formed and/or activated would reduce or prevent signal transduction and reduce the downstream proinflammatory effects of IL-17RA/IL-17RB activation. Such methods and antagonists would be useful in the treatment of various inflammation and autoimmune disorders that are influenced by the IL-17/IL-17R pathway. Embodiments of the invention are useful for in vitro assays to screen for antagonists or agonists of the IL-17RA-IL-17RB heteromeric receptor complex and/or to identify cells expressing the IL-17RA-IL-17RB heteromeric receptor complex.

Moreover, the knowledge that both IL-17RA and IL-17RB are required to form a functional IL-25 receptor complex leads to additional agents (such as antigen binding proteins) that are useful in inhibiting or antagonizing a biological activity of IL-25. For example, an antigen binding protein that binds to one or more subunits of the receptor complex (for example, an antibody that binds IL-17RA, or an antibody that binds IL-17RB) and inhibits binding or activation of the receptor complex by IL-25 will be a useful antagonist of IL-25.

In some embodiments, the antagonists of the invention are “isolated” or “substantially pure” (or “substantially homogeneous”) molecules. The term “isolated molecule” (where the molecule is, for example, a polypeptide, a peptide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or synthesized in a cellular system different from the cell from which it naturally originates, will be “isolated” from its naturally associated components.

A molecule also may be rendered substantially free of naturally associated components by isolation, using purification techniques well known in the art (i.e., a “purified” protein). Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

An “isolated” antagonist (i.e., a protein, a polypeptide, a peptide, or an antibody) is unaccompanied by at least some of the material with which it is normally associated in its natural state, in one embodiment constituting at least about 5%, in another embodiment at least about 50%, by weight, of the total protein in a given sample. A “substantially pure” protein comprises at least about 75% by weight of the total protein, with at least about 80% being specific, and at least about 90% being particularly specific. The definition includes the production of a protein from one organism in a different organism or host cell. Alternatively, the protein may be made at a significantly higher concentration than is normally seen, through the use of an inducible promoter or high expression promoter, such that the protein is made at increased concentration levels.

These are but a few of the many aspects of the various embodiments of the invention described herein.

### 1. IL-17RA-IL-17RB Antagonists

IL-17RA associates with IL-17RB to form a heteromeric receptor complex that is biologically active (that is, when activated by binding of ligand, a signal is transduced to a cell that results in a change in the biological activity of the cell, for example, induction of mRNAs, secretion of cytokines, change in morphology or activation state of the cell, etc.). An IL-17RA-IL-17RB heteromeric receptor complex is defined as an association (such as, but not limited to, protein-protein interactions) of at least IL-17RA and IL-17RB proteins displayed as a heteromeric receptor complex on the extracellular membrane of cells. This heteromeric receptor complex, at a minimum, is required for IL-25 signaling, i.e., IL-17RA and/or IL-17RB activation. It is understood that the IL-17RA-IL-17RB heteromeric receptor complex may further comprise additional proteins (i.e., “accessory” proteins). For example, a signalling molecule known as Act-1 is part of the IL-17A signalling cascade, and recent evidence indicates that it may be involved in IL-25 signalling as well (Claudio et al., J. Immunol. 182:1617, 2009; Swaidani et al., J. Immunol. 182:1631, 2009). IL-17RA-IL-17RB heteromeric receptor complex activation is effectuated through binding of IL-17 ligand family members, such as, but not limited to, IL-25 (IL-17E). IL-17RA-IL-17RB heteromeric receptor complex activation includes, but is not limited to, initiation of intracellular signaling cascade(s) and downstream events such as gene transcription and translation.

Embodiments are directed to antagonists, including antigen binding proteins, that inhibit the association of subunits (i.e., IL-17RA and IL-17RB and/or accessory proteins) in forming an IL-17RA -IL-17RB heteromeric receptor complex, as well as to antagonists (i.e., antigen binding proteins) that inhibit the binding of ligand (i.e., IL-25) to an IL-17RA-IL-17RB heteromeric receptor complex or subunit thereof. Additional embodiments are directed to antagonists (including antigen binding proteins) that bind to one or more subunits of an IL-17RA-IL-17RB heteromeric receptor complex and result in a conformational change that prevents association of the subunits of the complex, the binding of ligand thereto, or activation thereof.

“Antigen binding protein” as used herein is a protein that specifically binds an identified target protein (for example, a subunit of an 17RA-IL-17RB heteromeric receptor complex, or a heteromeric receptor complex itself). “Specifically binds” means that the antigen binding protein has higher affinity for the identified target protein than for another protein. Typically, “specifically binds” mean that the equilibrium dissociation constant is <10⁻⁷ to 10⁻¹¹ M, or <10⁻⁸ to <10⁻¹⁰ M, or <10⁻⁹ to <10⁻¹⁰ M.

Antigen binding proteins include an antibody, or fragment thereof, that specifically binds an identified target protein, as variously defined herein, as well as a peptide or polypeptide that specifically binds the identified target protein. Antigen binding proteins that inhibit the formation of an IL-17RA-IL-17RB heteromeric receptor complex or that inhibit the binding of ligand thereto or signaling thereby are referred to herein as IL-17RA-IL-17RB antagonists. Embodiments of IL-17RA-IL-17RB antagonists may thus bind to any part of the IL-17RA-IL-17RB heteromeric receptor complex (i.e., to the complex itself or to a subunit thereof) and inhibit receptor activation. Subgenera of the genus of IL-17RA-IL-17RB antagonists comprise antibodies, as variously defined herein, as well as peptides and polypeptides.

Activating or activation of a receptor is defined herein as the engagement of one or more intracellular signaling pathway(s) and the transduction of intracellular signaling (i.e., signal transduction) in response to a molecule binding to a membrane-bound receptor, such as but not limited to, a receptor: ligand interaction. Signal transduction, as used herein, is the relaying of a signal by conversion from one physical or chemical form to another; for example, in cell biology, the process by which a cell converts an extracellular signal into a response (such as cytokine secretion, proliferation or change in activation state of the cell).

“Inhibition” may be measured as a decrease in an activity of an IL-17RA-IL-17RB heteromeric receptor complex, for example, a decrease in the formation of a heteromeric receptor complex, a decrease in the binding of ligand (i.e. IL-17A IL-17F and/or IL-25) to a heteromeric receptor complex (or at least one subunit thereof), or a decrease in a biological activity in response to ligand such as IL-17A, IL-17F and/or IL-25 (i.e., stimulation of secretion of cytokines, changes in numbers or activation states of cells, or other biological effects) by at least 10%, 15%, 20%, 25%, 30%, 35%, 40%, 45%, 50%, 55%, 60%, 65%, 70%, 75%, 80%, 85%, 90%, 95%, or 100%. In one embodiment, the antagonists of the invention decrease an IL-17RA-IL-17RB heteromeric receptor complex activity by at least 30%, 40%, 50%, 60%, 70%, 80%, 90%, or 100%; in another embodiment, the antagonists of the invention inhibit an activity by at least 35%, 45%, 55%, 65%, 75%, 85%, 95% or more.

The inhibition of formation of a heteromeric receptor complex may be measured by any means known in the art, such as but not limited to the co-immunoprecipitation methods described herein. Other examples include Forster Resonance Energy Transfer (FRET) analysis and other methods that are known in the art and that can be used to quantitatively or qualitatively analyse ligand/receptor interaction. Inhibition of binding of ligand may also be measured by any means known in the art, such as FACS, EIA, RIA, the aforementioned assays, and methods that are known in the art for evaluating the interaction of two or more molecules, including those described herein and in U.S. Ser. No. 11/906,094.

In addition, “inhibition” may be measured as a loss of IL-25 activation of an IL-17RA-IL-17RB heteromeric receptor complex as measured by biologically relevant readout(s), such as but not limited to upregulated gene transcription (for example, increased levels of IL-5, IL-13, eotaxin, MCP-1, and/or IL-17RB mRNAs) and/or gene translation, and/or release of various factors associated with activation of the IL-17RA-IL-17RB heteromeric receptor complex, which includes IL-5 and/or IL-13, as well as any other proinflammatory mediator known in the art to be released from any cells expressing IL-17RA and/or IL-17RB. Additional biologically relevant readouts include changes in the numbers and/or appearance of cells in a biological sample (such as increased cellularity in bronchoalveolar lavage samples, goblet cell hyperplasia and/or vascular/perivascular inflammation in lung tissue samples).

Other embodiments of an IL-17RA-IL-17RB antagonist are directed to IL-17RA-IL-17RB antagonists that bind to IL-17RA. In one embodiment, the antagonists partially inhibit or fully inhibit association of subunits of IL-17RA-IL-17RB heteromeric receptor complex and thereby prevent heteromeric receptor complex formation. In some embodiments, the IL-17RA-IL-17RB antagonist need not block the binding of IL-25 to the IL-17RA-IL-17RB heteromeric receptor complex. In alternative embodiments, the IL-17RA-IL-17RB antagonist may block the binding of IL-25 to the IL-17RA-IL-17RB heteromeric receptor complex (or to a subunit thereof).

Additional embodiments of an IL-17RA-IL-17RB antagonist are directed to IL-17RA-IL-17RB antagonists that bind to IL-17RB. In one embodiment, the antagonists partially inhibit or fully inhibit association of subunits of IL-17RA-IL-17RB heteromeric receptor complex and thereby prevent heteromeric receptor complex formation. In some embodiments, the IL-17RA-IL-17RB antagonist need not block the binding of IL-25 to the IL-17RA-IL-17RB heteromeric receptor complex. In alternative embodiments, the IL-17RA-IL-17RB antagonist may block the binding of IL-25 to the IL-17RA-IL-17RB heteromeric receptor complex (or to a subunit thereof).

Further embodiments of an IL-17RA-IL-17RB antagonist are directed to IL-17RA-IL-17RB antagonists that bind to both IL-17RA and IL-17RB, including those that bind a heteromeric receptor complex. In one embodiment, the antagonists partially inhibit or fully inhibit association of subunits of IL-17RA-IL-17RB heteromeric receptor complex and thereby prevent heteromeric receptor complex formation. In some embodiments, the IL-17RA-IL-17RB antagonist need not block the binding of IL-25 to the IL-17RA-IL-17RB heteromeric receptor complex. In alternative embodiments, the IL-17RA-IL-17RB antagonist may block the binding of IL-25 to the IL-17RA-IL-17RB heteromeric receptor complex (or to a subunit thereof).

The various embodiments of IL-17RA-IL-17RB antagonists described above include IL-17RA-IL-17RB antagonists that bind to IL-17RA, or IL-17RB, or to the heteromeric receptor complex, and sterically inhibit or hinder the association of the subunits of the heteromeric receptor complex, thereby preventing IL-17RA-IL-17RB heteromeric receptor complex formation. In an example of steric hindrance of the association of subunits of the heteromeric receptor complex, the binding of an antagonist to a subunit occurs at a site that is required for association of that subunit with other subunits of the receptor complex, or near enough to the site that the spatial arrangement of the antagonist prevents association of the heteromeric receptor complex subunits. Alternatively, the various embodiments of IL-17RA-IL-17RB antagonists described above include IL-17RA-IL-17RB antagonists that bind to IL-17RA, or IL-17RB, or the heteromeric receptor complex, and induce (or prevent) a conformational alteration in one or more of the subunits of the heteromeric receptor complex, thereby inhibiting the formation of an IL-17RA-IL-17RB heteromeric receptor complex. In an example of conformational change preventing association of subunits of the heteromeric receptor complex, the binding of an antagonist to a subunit occurs at a site that may be distal from a site that is required for association of that subunit with other subunits of the receptor complex, and causes a change in the conformation of the subunit that prevents association thereof with other subunits, or prevents a conformational change that is necessary for association of the subunits. Similarly, the various embodiments of IL-17RA-IL-17RB antagonists described above include IL-17RA-IL-17RB antagonists that bind to IL-17RA, or IL-17RB, or the heteromeric receptor complex, and induce (or prevent) a conformational alteration that inhibits signal transduction, or that sterically hinder signal transduction by the heteromeric receptor complex.

In another alternative embodiment, the various IL-17RA-IL-17RB antagonists described above include IL-17RA-IL-17RB antagonists that bind to IL-17RA, or IL-17RB, or the heteromeric receptor complex, and induce a conformational alteration in the heteromeric receptor complex (or a subunit thereof) and thereby inhibit the binding of IL-25 (or another ligand) to the IL-17RA-IL-17RB heteromeric receptor complex. The embodiments further include IL-17RA-IL-17RB antagonists that bind to IL-17RA, or IL-17RB, or both IL-17RA and IL-17RB and sterically hinder or inhibit the binding of ligand (such as IL-25) to the IL-17RA-IL-17RB heteromeric receptor complex. Also included in the present embodiments are antagonists that bind to IL-17RA, or IL-17RB, or both IL-17RA and IL-17RB and inhibit (partially or fully) a signaling pathway of the receptor complex, thereby inhibit signaling via the IL-17RA-IL-17RB heteromeric receptor complex.

In an additional alternative embodiment, an IL-17RA-IL-17RB antagonist binds to a ligand (i.e., IL-17A, IL-25, etc), and inhibits signalling via IL-17RA-IL-17RB heteromeric receptor complex. Such antagonists may act by inhibiting binding to one subunit of a IL-17RA-IL-17RB heteromeric receptor complex, or to more than one such subunit. Thus, for example, an antagonist may permit a ligand to bind a first receptor subunit, but prevent interaction of a second receptor subunit to either the ligand or to the first receptor subunit. Such inhibition may occur as described above, for example by steric hindrance of binding, induction of a conformational alteration, etc. in such a way as to inhibit (partially or fully) signaling via the IL-17RA-IL-17RB heteromeric receptor complex.

### 1.1 IL-17RA-IL-17RB Antagonists: Antibodies

Embodiments of IL-17RA-IL-17RB antagonists comprise antibodies, or fragments thereof, as variously defined herein. Accordingly, the IL-17RA-IL-17RB antagonists include polyclonal antibodies, monoclonal antibodies, bispecific antibodies, diabodies, minibodies, domain antibodies, synthetic antibodies (sometimes referred to herein as “antibody mimetics”), chimeric antibodies, humanized antibodies, fully human antibodies, antibody fusions (sometimes referred to as “antibody conjugates”), as well as fragments thereof.

IL-17RA-IL-17RB antagonist antibodies may also comprise single-domain antibodies that comprise dimers of two heavy chains and include no light chains, such as those found in camels and llamas (see, for example Muldermans, et al., 2001, J. Biotechnol. 74:277-302; Desmyter, et al., 2001, J. Biol. Chem. 276:26285-26290).

IL-17RA-IL-17RB antagonist antibodies may comprise a tetramer, or fragments thereof. Each tetramer is typically composed of two identical pairs of polypeptide chains, each pair having one “light” (typically having a molecular weight of about 25 kDa) and one “heavy” chain (typically having a molecular weight of about 50-70 kDa). The amino-terminal portion of each chain includes a variable region is primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. IL-17RA-IL-17RB antagonist antibodies include all such isotypes. For exemplary purposes, antibody fragments include but are not limited to, F(ab), F(ab′), F(ab′)2, Fv, and single chain Fv fragments (scfv), as well as single-chain antibodies. IL-17RA-IL-17RB antagonist antibodies may comprise any of the foregoing examples.

The structure of antibodies is well known in the art and need not be reproduced here, but by way of example, the variable regions of the heavy and light chains typically exhibit the same general structure of relatively conserved framework regions (FR) joined by three hypervariable regions, also called complementarity determining regions or CDRs. The CDRs are the hypervariable regions of an antibody (or antigen binding protein, as outlined herein), that are responsible for antigen recognition and binding. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. In some embodiments, the assignment of amino acids to each domain may be in accordance with the definitions of Kabat Sequences of Proteins of Immunological Interest. See, Chothia, et al., 1987, J. Mol. Biol. 196:901-917; Chothia, et al., 1989, Nature 342:878-883.

A “complementary determining region” or “CDR,” as used herein, refers to a binding protein region that constitutes the major surface contact points for antigen binding. A binding protein of the invention may have six CDRs, for example one heavy chain CDR1 (“CDRH1”), one heavy chain CDR2 (“CDRH2”), one heavy chain CDR3 (“CDRH3”), one light chain CDR1 (“CDRL1”), one light chain CDR2 (“CDRL2”), one light chain CDR3 (“CDRL3”). CDRH1 typically comprises about five (5) to about seven (7) amino acids, CDRH2 typically comprises about sixteen (16) to about nineteen (19) amino acids, and CDRH3 typically comprises about three (3) to about twenty five (25) amino acids. CDRL1 typically comprises about ten (10) to about seventeen (17) amino acids, CDRL2 typically comprises about seven (7) amino acids, and CDRL3 typically comprises about seven (7) to about ten (10) amino acids

At a minimum, an IL-17RA-IL-17RB antagonist antibody comprises all or part of a light or heavy chain variable region, or all or part of both a light and heavy chain variable region that specifically binds to IL-17RA, or IL-17RB, or both IL-17RA and IL-17RB. Examples of fragments (i.e., “part”) of variable regions comprise the CDRs. Stated differently, at a minimum, an IL-17RA-IL-17RB antagonist antibody comprises at least one CDR of a variable region, wherein the CDR specifically binds IL-17RA, or IL-17RB, or both IL-17RA and IL-17RB. In alternative embodiments, an IL-17RA-IL-17RB antagonist antibody comprises at least two, or at least three, or at least four, or at least five, or at least all six CDRs of a/the variable region(s), wherein at least one of the CDRs specifically binds IL-17RA, or IL-17RB, or both IL-17RA and IL-17RB. The CDR may be from a heavy or light chain, and may be one of any of the three CDRs within each chain, that is, the CDRs are each independently selected from CDRH1, CDRH2, CDRH3, CDRL1, CDRL2 and CDRL3.

Embodiments of the IL-17RA-IL-17RB antagonist antibodies may comprise a scaffold structure into which useful CDR(s) are grafted. Some embodiments include human scaffold components for humanized antibodies. In one embodiment, the scaffold structure is a traditional, tetrameric antibody structure. Thus, embodiments of the IL-17RA-IL-17RB antagonist antibodies may include the additional components such as framework, J and D regions, constant regions, etc. that make up a heavy or light chain. Embodiments of the IL-17RA-IL-17RB antagonist antibodies may comprise antibodies that have a modified Fc domain, referred to as an Fc variant. An “Fc variant” refers to a molecule or sequence that is modified from a native Fc but still comprises a binding site for the salvage receptor, FcRn. Other examples of an “Fc variant” include a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

Embodiments of IL-17RA-IL-17RB antagonist antibodies comprise human monoclonal antibodies. Human monoclonal antibodies directed against human IL-17RA, or IL-17RB, or both IL-17RA and IL-17RB may be made using any known methods known in the art, such as but not limited to XenoMouse™ technology (see, for example U.S. Pat. Nos. 6,114,598; 6,162,963; 6,833,268; 7,049,426; 7,064,244; Green et al, 1994, Nature Genetics 7:13-21; Mendez et al., 1997, Nature Genetics 15:146-156; Green and Jakobovitis, 1998, J. Ex. Med. 188:483-495). Other examples of making fully human antibodies include UltiMab Human Antibody Development System™ and Trans-Phage Technology™ (Medarex Corp., Princeton, N.J.), phage-display technologies, ribosome-display technologies (see for example Cambridge Antibody Technology, Cambridge, UK), as well as any other method known in the art.

Certain embodiments of IL-17RA-IL-17RB antagonist antibodies comprise chimeric and humanized antibodies, or fragments thereof. In general, both chimeric antibodies and humanized antibodies refer to antibodies that combine regions from more than one species. For example, chimeric antibodies traditionally comprise variable region(s) from a non-human species and the constant region(s) from a human. Humanized antibodies generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is well known in the art (see, for example Jones, 1986, Nature 321:522-525; Verhoeyen et al., 1988, Science 239:1534-1536). Humanized antibodies can also be generated using mice with a genetically engineered immune system or by any other method or technology known in the art (see for example Roque, et al., 2004, Biotechnol. Prog. 20:639-654). In some embodiments, the CDRs are human, and thus both humanized and chimeric antibodies in this context can include some non-human CDRs; for example, humanized antibodies may be generated that comprise the CDRH3 and CDRL3 regions, with one or more of the other CDR regions being of a different special origin.

In one embodiment, the IL-17RA-IL-17RB antagonist antibodies comprise a multispecific antibody. These are antibodies that bind to two (or more) different antigens. An example of a bispecific antibody known in the art are “diabodies”. Diabodies can be manufactured in a variety of ways known in the art, e.g., prepared chemically or from hybrid hybridomas (Holliger and Winter, 1993, Current Opinion Biotechnol. 4:446-449). A specific embodiment of a multispecific IL-17RA-IL-17RB antagonist antibody is an antibody that has the capacity to bind to both IL-17RA and IL-17RB.

In alternative embodiments, the IL-17RA-IL-17RB antagonist antibodies comprise a minibody. Minibodies are minimized antibody-like proteins comprising a single chain Fv (scFv; described below) joined to a CH3 domain (see, for example Hu, et al., 1996, Cancer Res. 56:3055-3061).

In alternative embodiments, the IL-17RA-IL-17RB antagonist antibodies comprise a domain antibody; for example those described in U.S. Pat. No. 6,248,516. Domain antibodies (dAbs) are functional binding domains of antibodies, corresponding to the variable regions of either the heavy (VH) or light (VL) chains of human antibodies. dAbs have a molecular weight of approximately 13 kDa, or less than one-tenth the size of a full antibody. dAbs are well expressed in a variety of hosts including bacterial, yeast, and mammalian cell systems. In addition, dAbs are highly stable and retain activity even after being subjected to harsh conditions, such as freeze-drying or heat denaturation. See, for example, U.S. Pat. Nos. 6,291,158; 6,582,915; 6,593,081; 6,172,197; U.S. Ser. No. 2004/0110941; European Patent 0368684; U.S. Pat. No. 6,696,245, WO04/058821, WO04/003019 and WO03/002609.

As mentioned previously, the IL-17RA-IL-17RB antagonist antibodies may comprise an antibody fragment, i.e., a fragment of any of the antibodies mentioned herein that retain binding specificity to IL-17RA, or IL-17RB, or both IL-17RA and IL-17RB. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (see for example Ward, et al., 1989, Nature 341:544-546) which consists of a single variable, (v) isolated CDR regions, (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (see, for example Bird, et al., 1988 Science 242:423-426; Huston, et al., 1988, Proc. Natl. Acad. Sci. 85:5879-5883), (viii) bispecific single chain Fv dimers, and (ix) “diabodies” or “triabodies”, multivalent or multispecific fragments constructed by gene fusion (see, for example, Tomlinson, et. al., 2000, Methods Enzymol. 326:461-479; WO94/13804; Holliger, et al., 1993, Proc. Natl. Acad. Sci. 90:6444-6448). The antibody fragments may be modified. For example, the molecules may be stabilized by the incorporation of disulphide bridges linking the VH and VL domains (see, for example, Reiter, et al., 1996, Nature Biotech. 14:1239-1245). Again, as outlined herein, the non-CDR components of these fragments are preferably human sequences.

In further embodiments, the IL-17RA-IL-17RB antagonist antibodies comprise an antibody fusion protein (sometimes referred to herein as an “antibody conjugate”). The conjugate partner can be proteinaceous or non-proteinaceous; the latter generally being generated using functional groups on the antigen binding protein (see the discussion on covalent modifications of the antigen binding proteins) and on the conjugate partner. For example linkers are known in the art; for example, homo- or hetero-bifunctional linkers as are well known (see, for example, 1994 Pierce Chemical Company catalog, technical section on cross-linkers, pages 155-200, incorporated herein by reference). Suitable conjugates include, but are not limited to, labels as described below, drugs and cytotoxic agents including, but not limited to, cytotoxic drugs (e.g., chemotherapeutic agents) or toxins or active fragments of such toxins. Suitable toxins and their corresponding fragments include diptheria A chain, exotoxin A chain, ricin A chain, abrin A chain, curcin, crotin, phenomycin, enomycin and the like. Cytotoxic agents also include radiochemicals made by conjugating radioisotopes to antigen binding proteins, or binding of a radionuclide to a chelating agent that has been covalently attached to the antigen binding protein. Additional embodiments utilize calicheamicin, auristatins, geldanamycin and maytansine.

In one embodiment, the IL-17RA-IL-17RB antagonist antibodies comprise an antibody analog, sometimes referred to as “synthetic antibodies.” For example, a variety of alternative protein scaffolds or artificial scaffolds may be grafted with CDRs from IL-17RA-IL-17RB antagonist antibodies. Such scaffolds include, but are not limited to, mutations introduced to stabilize the three-dimensional structure of the binding protein as well as wholly synthetic scaffolds consisting for example of biocompatible polymers. See, for example, Korndorfer, et al., 2003, Proteins: Structure, Function, and Bioinformatics, Volume 53, Issue 1:121-129; Roque, et al., 2004, Biotechnol. Prog. 20:639-654. In alternative embodiments the IL-17RA-IL-17RB antagonist antibodies may comprise peptide antibody mimetics, or “PAMs”, as well as antibody mimetics utilizing fibronection components as a scaffold.

### 1.2 IL-17RA-IL-17RB Antagonists: Peptides/Polypeptides

Embodiments of IL-17RA-IL-17RB antagonists comprise proteins in the form of peptides and polypeptides that specifically bind to IL-17RA, or IL-17RB, or both IL-17RA and IL-17RB, that inhibit an activity of IL-17A, IL-17F and/or IL-25. In some embodiments, IL-17RA-IL-17RB antagonists inhibit the association of the subunits of the IL-17RA-IL-17RB heteromeric receptor complex; induce (or prevent) a conformational change in the receptor subunits thereby inhibiting their interaction; inhibit the binding of ligand (i.e., IL-25) to the heteromeric receptor complex (or a subunit thereof) or induce a conformational change in the heteromeric receptor complex (or a subunit thereof) that inhibits the binding of ligand thereto.

Embodiments include recombinant IL-17RA-IL-17RB antagonists. A “recombinant protein” is a protein made using recombinant techniques, i.e., through the expression of a recombinant nucleic acid using methods known in the art.

A “peptide,” as used herein refers to molecules of 1 to 100 amino acids. Exemplary peptides that bind to IL-17RA, or IL-17RB, or both IL-17RA and IL-17RB that inhibit the association of IL-17RA and IL-17RB in forming an IL-17RA-IL-17RB heteromeric receptor complex or inhibit IL-17RA-IL-17RB heteromeric receptor complex signaling may comprise those generated from randomized libraries. For example, peptide sequences from fully random sequences (e.g., selected by phage display methods or RNA-peptide screening) and sequences in which one or more residues of a naturally occurring molecule is replaced by an amino acid residue not appearing in that position in the naturally occurring molecule. Exemplary methods for identifying peptide sequences include phage display, E. coli display, ribosome display, RNA-peptide screening, chemical screening, and the like.

By “protein,” as used herein, is meant at least two covalently attached amino acids, which includes proteins, polypeptides, oligopeptides and peptides. In some embodiments, the two or more covalently attached amino acids are attached by a peptide bond. The protein may be made up of naturally occurring amino acids and peptide bonds, for example when the protein is made recombinantly using expression systems and host cells, as outlined below. Alternatively, in some embodiments (for example when proteinaceous candidate agents are screened for the ability to inhibit IL-17RA and IL-17RB association) the protein may include synthetic amino acids (e.g., homophenylalanine, citrulline, ornithine, and norleucine), or peptidomimetic structures, i.e., “peptide or protein analogs”, such as peptoids (see, Simon et al., 1992, Proc. Natl. Acad. Sci. U.S.A. 89:9367, incorporated by reference herein), which can be resistant to proteases or other physiological and/or storage conditions. Such synthetic amino acids may be incorporated in particular when the protein is synthesized in vitro by conventional methods well known in the art. In addition, any combination of peptidomimetic, synthetic and naturally occurring residues/structures can be used. “Amino acid” also includes imino acid residues such as proline and hydroxyproline. The amino acid “R group” or “side chain” may be in either the (L)- or the (S)-configuration. In a specific embodiment, the amino acids are in the (L)- or (S)-configuration.

One example of an antagonistic protein is a soluble IL-17RA-IL-17RB heteromeric receptor. Methods of preparing such soluble heteromeric receptors are known in the art, and are described, for example, in U.S. Pat. No. 6,589,764, issued Jul. 8, 2003, incorporated by reference herein. IL-17A-IL-17B receptor complexes include IL-17RA and IL-17RB (and/or additional subunits) as proteins coexpressed in the same cell, or as receptor subunits linked to each other (for example, via covalent linkages by any suitable means, such as via a cross-linking reagent or a polypeptide linker). In one embodiment, a heteromeric receptor is formed from a fusion protein of each receptor component with a portion of an antibody molecule, such as an Fc region. Alternatively, the heteromeric IL-17A-IL-17B receptor may be formed through non-covalent interactions, such as that of biotin with avidin.

### 2.0 IL-17RA-IL-17RB Antagonists

As mentioned above, IL-17RA-IL-17RB antagonists include IL-17RA-IL-17RB antigen binding proteins, which includes, but is not limited to, antibodies, peptides, and polypeptides, as well as other antagonists (including other polypeptides or proteins). Alternative embodiments of IL-17RA-IL-17RB antagonists (e.g., IL-17RA-IL-17RB antigen binding proteins) comprise covalent modifications of IL-17RA-IL-17RB antagonists. Such modifications may be done post-translationally. For example, several types of covalent modifications of the IL-17RA-IL-17RB antagonists are introduced into the molecule by reacting specific amino acid residues of the antagonist with an organic derivatizing agent that is capable of reacting with selected side chains or the N- or C-terminal residues. The following represent examples of such modifications to the IL-17RA-IL-17RB antagonists.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in IL-17RAdioimmunoassay, the chloramine T method described above being suitable. Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Derivatization with bifunctional agents is useful for crosslinking IL-17RA-IL-17RB antagonists to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W. H. Freeman & Co., San Francisco, pp. 79-86 [1983]), acetylation of the N-terminal amine, and amidation of any C-terminal carboxyl group.

Another type of covalent modification of the IL-17RA-IL-17RB antagonists included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used. Addition of glycosylation sites to the IL-17RA-IL-17RB antagonists is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the IL-17RA-IL-17RB antagonists is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston, 1981, CRC Crit. Rev. Biochem., pp. 259-306.

Removal of carbohydrate moieties present on the starting IL-17RA-IL-17RB antagonists may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Another type of covalent modification of the IL-17RA-IL-17RB antagonists comprises linking the antigen binding protein to various nonproteinaceous polymers, including, but not limited to, various polyols such as polyethylene glycol (PEG), polypropylene glycol or polyoxyalkylenes, in the manner set forth in U.S. Pat. No. 4,640,835; 4,496,689; 4,301,144; 4,670,417; 4,791,192 or 4,179,337. In addition, as is known in the art, amino acid substitutions may be made in various positions within the antigen binding protein to facilitate the addition of polymers such as PEG.

Covalent modifications of IL-17RA-IL-17RB antagonists are included within the scope of this invention, and are generally, but not always, done post-translationally. For example, several types of covalent modifications of the IL-17RA-IL-17RB antagonists are introduced into the molecule by reacting specific amino acid residues of the IL-17RA-IL-17RB antagonists with an organic derivatizing agent that is capable of reacting with selected side chains or the N- or C-terminal residues.

In some embodiments, the covalent modification of the antigen binding proteins of the invention comprises the addition of one or more labels. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, beta-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labeling group is coupled to the antigen binding protein via spacer arms of various lengths to reduce potential steric hindrance. Various methods for labelling proteins are known in the art and may be used in performing the present invention.

Specific labels include optical dyes, including, but not limited to, chromophores, phosphors and fluorophores, with the latter being specific in many instances. Fluorophores can be either “small molecule” fluores, or proteinaceous fluores. By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malachite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), f3 galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

All of the above-described modifications of antigen binding proteins could also be made to any other proteinaceous IL-17RA-IL-17RB antagonist, for example a heteromeric IL-17RA-IL-17RB complex, or a polypeptide or peptide antagonist as described herein.

### 3.0 Methods of Use

The present invention also provides methods of using IL-17RA-IL-17RB antagonists, including for example, use of IL-17RA-IL-17RB antagonists for diagnostic purposes, or for treatment purposes. It is understood that, for treatment, use of IL-17RA-IL-17RB antagonists is generally for the reduction or amelioration of signs and/or symptoms of the disease or condition for which treatment is given. The invention provides IL-17RA-IL-17RB antagonists as described throughout this specification that may be used in the preparation or manufacture of a medicament for the treatment of various conditions and diseases described herein. Additionally, an effective amount of an IL-17RA-IL-17RB antagonist and a therapeutically effective amount of one or more additional active agents as described herein may be used in the preparation or manufacture of a medicament useful for the described treatments. Some embodiments include a kit of parts comprising an IL-17RA-IL-17RB antagonist; optionally, such a kit may include at least one additional active ingredient for separate, simultaneous or subsequent administration to a subject in need thereof.

Additional embodiments include methods of inhibiting IL-17RA and/or IL-17RB activation in cells expressing IL-17RA and IL-17RB using one or more of the IL-17RA-IL-17RB antagonists described herein. For example, a method of inhibiting IL-17RA and/or IL-17RB activation in cells expressing IL-17RA and IL-17RB comprises exposing said cells to an IL-17RA-IL-17RB antagonist, wherein the IL-17RA-IL-17RB antagonist binds at least one subunit or component of the heteromeric receptor complex and partially inhibits or fully inhibits association thereof with another subunit or component of the heteromeric receptor complex (either via steric hindrance or conformational change) thereby preventing IL-17RA-IL-17RB heteromeric receptor complex formation. In some embodiments, the IL-17RA-IL-17RB antagonist binds one subunit of the heteromeric receptor complex. In alternative embodiments, the IL-17RA-IL-17RB antagonist binds more than one subunit of the heteromeric receptor complex, or binds the heteromeric receptor complex itself. In some embodiments, the IL-17RA-IL-17RB antagonist need not inhibit the binding of ligand (such as IL-25) to one or more components of the heteromeric receptor complex to inhibit IL-17RA and/or IL-17RB activation. In alternative embodiments, the IL-17RA-IL-17RB antagonist inhibits the binding of ligand (i.e., IL-25) to IL-17RA and/or IL-17RB, and inhibits IL-17RA and/or IL-17RB activation. Additional embodiments comprise a method wherein said IL-17RA-IL-17RB antagonist is an antigen binding protein, as defined herein; optionally the antigen binding protein is in the form of a pharmaceutical composition.

Further embodiments include methods of inhibiting IL-17RA and/or IL-17RB activation in cells expressing at least IL-17RA and IL-17RB in vivo using one or more of the IL-17RA-IL-17RB antagonists described herein. For example, a method of inhibiting IL-17RA and/or IL-17RB activation in cells expressing IL-17RA and IL-17RB in vivo comprises exposing said cells to an IL-17RA-IL-17RB antagonist, wherein the IL-17RA-IL-17RB antagonist binds at least one subunit or component of the heteromeric receptor complex and partially inhibits or fully inhibits association thereof with another subunit or component of the heteromeric receptor complex (either via steric hindrance or conformational change) thereby inhibiting IL-17RA-IL-17RB heteromeric receptor complex activation. In some embodiments, the IL-17RA-IL-17RB antagonist binds one subunit of the heteromeric receptor complex. In alternative embodiments, the IL-17RA-IL-17RB antagonist binds more than one subunit of the heteromeric receptor complex, or binds the heteromeric receptor complex itself. In some embodiments, the IL-17RA-IL-17RB antagonist need not block the binding of ligand (such as IL-25) to one or more components of the heteromeric receptor complex to inhibit IL-17RA and/or IL-17RB activation. In alternative embodiments, the IL-17RA-IL-17RB antagonist inhibits the binding of ligand (i.e., IL-25) to IL-17RA and/or IL-17RB, and inhibits IL-17RA and/or IL-17RB activation. Additional embodiments comprise a method wherein said IL-17RA-IL-17RB antagonist is an antigen binding protein, as defined herein; optionally the antigen binding protein is in the form of a pharmaceutical composition.

Additional embodiments include methods of reducing proinflammatory mediators released after IL-17RA-IL-17RB heteromeric receptor complex activation in cells expressing said complex in vivo using one or more of the IL-17RA-IL-17RB antagonists described herein. For example, a method of reducing release of proinflammatory mediators after IL-17RA-IL-17RB heteromeric receptor complex activation in cells expressing said complex in vivo comprises exposing said cells to an IL-17RA-IL-17RB antagonist, wherein the IL-17RA-IL-17RB antagonist binds at least one subunit or component of the heteromeric receptor complex and partially inhibits or fully inhibits formation or activation of an IL-17RA-IL-17RB heteromeric receptor complex heteromeric receptor complex (either via steric hindrance or conformational change) thereby reducing release of proinflammatory mediators. In some embodiments, the IL-17RA-IL-17RB antagonist binds one subunit of the heteromeric receptor complex. In alternative embodiments, the IL-17RA-IL-17RB antagonist binds more than one subunit of the heteromeric receptor complex, or binds the heteromeric receptor complex itself. In some embodiments, the IL-17RA-IL-17RB antagonist need not inhibit the binding of ligand (such as IL-25) to one or more components of the heteromeric receptor complex to reduce release of proinflammatory mediators. In alternative embodiments, the IL-17RA-IL-17RB antagonist inhibits the binding of ligand (i.e., IL-25) to IL-17RA and/or IL-17RB, and reduces release of proinflammatory mediators. Additional embodiments comprise a method wherein said IL-17RA-IL-17RB antagonist is an antigen binding protein, as defined herein; optionally the antigen binding protein is in the form of a pharmaceutical composition.

Additional embodiments comprise methods, as described above, wherein the proinflammatory mediator is at least one of the following: IL-5, IL-6, IL-8, IL-12, IL-13, CXCL1, CXCL2, GM-CSF, G-CSF, M-CSF, IL-1β, TNFα, RANK-L, LIF, PGE2, MMP3, MMP9, GROα, NO, eotaxin, MCP-1, and IL-17RB, as well as any other proinflammatory mediator known in the art to be released from any cells through activation of IL-17RA and/or IL-17RB.

Further embodiments include methods, as described above, of treating IL-17 family member-associated disorders, such as but not limited to, inflammatory and autoimmune disorders with the IL-17RA-IL-17RB antagonists.

Additional embodiments include methods of treating inflammation, wherein the IL-17RA-IL-17RB heteromeric receptor complex is partially or fully blocked from being activated by administering one or more of the IL-17RA-IL-17RB antagonists described herein. For example, a method of treating inflammation in a patient in need thereof comprises administering to said patient an IL-17RA-IL-17RB antagonist, wherein the IL-17RA-IL-17RB antagonist binds at least one subunit or component of the heteromeric receptor complex and partially inhibits or fully inhibits formation or activation of the heteromeric receptor complex (either via steric hindrance or conformational change) thereby facilitating treatment of inflammation. In some embodiments, the IL-17RA-IL-17RB antagonist binds one subunit of the heteromeric receptor complex. In alternative embodiments, the IL-17RA-IL-17RB antagonist binds more than one subunit of the heteromeric receptor complex, or binds the heteromeric receptor complex itself. In some embodiments, the IL-17RA-IL-17RB antagonist need not block the binding of ligand (such as IL-25) to one or more components of the heteromeric receptor complex to be useful in treating inflammation. In alternative embodiments, the IL-17RA-IL-17RB antagonist inhibits the binding of ligand (i.e., IL-25) to IL-17RA and/or IL-17RB, and is useful in treating inflammation. Additional embodiments comprise a method wherein said IL-17RA-IL-17RB antagonist is an antibody, as defined herein; optionally the antibody is in the form of a pharmaceutical composition.

Further embodiments include methods of treating an autoimmune disorder, wherein the IL-17RA-IL-17RB heteromeric receptor complex is partially or fully blocked from being activated by administering one or more of the IL-17RA-IL-17RB antagonists described herein. For example, a method of treating an autoimmune disorder in a patient in need thereof comprises administering to said patient an IL-17RA-IL-17RB antagonist, wherein the IL-17RA-IL-17RB antagonist binds at least one subunit or component of the heteromeric receptor complex and partially inhibits or fully inhibits formation or activation of the heteromeric receptor complex thereby facilitating treatment of the autoimmune disorder. In some embodiments, the IL-17RA-1-17RB antagonist binds one subunit of the heteromeric receptor complex. In alternative embodiments, the IL-17RA-IL-17RB antagonist binds more than one subunit of the heteromeric receptor complex, or binds the heteromeric receptor complex itself. In some embodiments, the IL-17RA-IL-17RB antagonist need not block the binding of ligand (such as IL-25) to one or more components of the heteromeric receptor complex to be useful in treatment of an autoimmune disorder. In alternative embodiments, the IL-17RA-IL-17RB antagonist inhibits the binding of ligand (i.e., IL-25) to IL-17RA and/or IL-17RB, and is useful in treatment of an autoimmune disorder. Additional embodiments comprise a method wherein said IL-17RA-IL-17RB antagonist is an antibody, as defined herein; optionally the antibody is in the form of a pharmaceutical composition.

Additional embodiments include methods of treating inflammation and/or autoimmune disorders, as described above, wherein the disorders include, but are not limited to, cartilage inflammation, and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma (including extrinsic and intrinsic asthma as well as related chronic inflammatory conditions, or hyperresponsiveness, of the airways), chronic obstructive pulmonary disease (COPD. i.e., chronic bronchitis, emphysema), Acute Respiratory Disorder Syndrome (ARDS), respiratory distress syndrome, cystic fibrosis, pulmonary hypertension, pulmonary vasoconstriction, acute lung injury, allergic bronchopulmonary aspergillosis, hypersensitivity pneumonia, eosinophilic pneumonia, bronchitis, allergic bronchitis bronchiectasis, tuberculosis, hypersensitivity pneumonitis, occupational asthma, asthma-like disorders, sarcoid, reactive airway disease (or dysfunction) syndrome, byssinosis, interstitial lung disease, hyper-eosinophilic syndrome, rhinitis, sinusitis, and parasitic lung disease, airway hyperresponsiveness associated with viral-induced conditions (for example, respiratory syncytial virus (RSV), parainfluenza virus (PIV), rhinovirus (RV) and adenovirus), Guillain-Barre disease, Type I diabetes mellitus, Graves' disease, Addison's disease, Raynaud's phenomenon, autoimmune hepatitis, GVHD, and the like.

Additional embodiments include pharmaceutical compositions comprising a therapeutically effective amount of one or more of an IL-17RA-IL-17RB antagonist together with a pharmaceutically acceptable diluent, carrier, solubilizer, emulsifier, preservative, and/or adjuvant. In addition, the invention provides methods of treating a patient by administering such pharmaceutical composition as well as methods for preparing or manufacturing a medicament for use in treating the afore-mentioned conditions.

Acceptable formulation materials are nontoxic to recipients at the dosages and concentrations employed. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the IL-17RA-IL-17RB antagonist. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute. In certain embodiments, IL-17RA-IL-17RB antagonist compositions may be prepared for storage by mixing the selected composition having the desired degree of purity with optional formulation agents (REMINGTON′S PHARMACEUTICAL SCIENCES, supra) in the form of a lyophilized cake or an aqueous solution. Further, in certain embodiments, the IL-17RA-IL-17RB antagonist product may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the IL-17RA-IL-17RB antagonists may be provided in the form of a pyrogen-free, parenterally acceptable aqueous solution comprising the desired IL-17 receptor antigen binding protein in a pharmaceutically acceptable vehicle. A particularly suitable vehicle for parenteral injection is sterile distilled water in which the IL-17RA-IL-17RB antagonist is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Pharmaceutical compositions of the invention can be formulated for inhalation. In these embodiments, IL-17RA-IL-17RB antagonist may be formulated as a dry, inhalable powder. Inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins.

It is also contemplated that formulations can be administered orally. IL-17RA-IL-17RB antagonists that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the IL-17RA-IL-17RB antagonist. Diluents, flavorings, low melting point waxes, vegetable oils, lubricants, suspending agents, tablet disintegrating agents, and binders may also be employed.

A pharmaceutical composition of the invention is preferably provided to comprise an effective quantity of one or more IL-17RA-IL-17RB antagonists in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving IL-17RA-IL-17RB antagonists in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer, et al., 1981, J. Biomed. Mater. Res. 15:167-277 and Langer, 1982, Chem. Tech. 12:98-105), ethylene vinyl acetate (Langer, et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein, et al., 1985, Proc. Natl. Acad. Sci. U.S.A. 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The therapeutically effective amount of an IL-17RA-IL-17RB antagonist-containing pharmaceutical composition to be employed will depend, for example, upon the therapeutic context and objectives. One skilled in the art will appreciate that the appropriate dosage levels for treatment will vary depending, in part, upon the molecule delivered, the indication for which the IL-17RA-IL-17RB antagonist is being used, the route of administration, and the size (body weight, body surface or organ size) and/or condition (the age and general health) of the patient. In certain embodiments, the clinician may titer the dosage and modify the route of administration to obtain the optimal therapeutic effect. A typical dosage may range from about 0.1 μg/kg to up to about 30 mg/kg or more, depending on the factors mentioned above. In specific embodiments, the dosage may range from 0.1 μg/kg up to about 30 mg/kg, optionally from 1 μg/kg up to about 30 mg/kg or from 10 μg/kg up to about 5 mg/kg. Of course, it is understood that this is to be determined by qualified physicians and that these doses are merely exemplary. Dosing frequency will depend upon the pharmacokinetic parameters of the particular IL-17RA-IL-17RB antagonist in the formulation used. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In certain embodiments, the IL-17RA-IL-17RB antagonists can be administered to patients throughout an extended time period. Chronic administration of an IL-17RA-IL-17RB antagonist may minimize the adverse immune or allergic response commonly associated with IL-17RA-IL-17RB antagonist that are not fully human, for example an antibody raised against a human antigen in a non-human animal, for example, a non-fully human antibody or non-human antibody produced in a non-human species.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

The composition also may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

The IL-17RA-IL-17RB antagonists described herein may be used in combination (pre-treatment, post-treatment, or concurrent treatment) with pharmaceutical agents used in treating the diseases and conditions described herein. In one embodiment, the IL-17RA-IL-17RB antagonists described herein may be used in combination (pre-treatment, post-treatment, or concurrent treatment) with any of one or more TNF inhibitors for the treatment or prevention of the diseases and disorders recited herein, such as but not limited to, all forms of soluble TNF receptors including Etanercept (such as ENBREL®), as well as all forms of monomeric or multimeric p75 and/or p55 TNF receptor molecules and fragments thereof; anti-human TNF antibodies, such as but not limited to, Infliximab (such as REMICADE®), and D2E7 (such as HUMIRA®), and the like. Such TNF inhibitors include compounds and proteins which block in vivo synthesis or extracellular release of TNF. In a specific embodiment, the present invention is directed to the use of an IL-17RA-IL-17RB antagonist in combination (pre-treatment, post-treatment, or concurrent treatment) with any of one or more of the following TNF inhibitors: TNF binding proteins (soluble TNF receptor type-I and soluble TNF receptor type-II (“sTNFRs”), as defined herein), anti-TNF antibodies, granulocyte colony stimulating factor; thalidomide; BN 50730; tenidap; E 5531; tiapafant PCA 4248; nimesulide; panavir; rolipram; RP 73401; peptide T; MDL 201,449A; (1R,3S)-Cis-1-[9-(2,6-diaminopurinyl)]-3-hydroxy-4-cyclopentene hydrochloride; (1R,3R)-trans-1-(9-(2,6-diamino)purine]-3-acetoxycyclopentane; (1R,3R)-trans-1-[9-adenyl)-3-azidocyclopentane hydrochloride and (1R,3R)-trans-1-(6-hydroxy-purin-9-yl)-3-azidocyclopentane. TNF binding proteins are disclosed in the art (EP 308 378, EP 422 339, GB 2 218 101, EP 393 438, WO 90/13575, EP 398 327, EP 412 486, WO 91/03553, EP 418 014, JP 127,800/1991, EP 433 900, U.S. Pat. No. 5,136,021, GB 2 246 569, EP 464 533, WO 92/01002, WO 92/13095, WO 92/16221, EP 512 528, EP 526 905, WO 93/07863, EP 568 928, WO 93/21946, WO 93/19777, EP 417 563, WO 94/06476, and PCT International Application No. PCT/US97/12244). For example, EP 393 438 and EP 422 339 teach the amino acid and nucleic acid sequences of a soluble TNF receptor type I (also known as “sTNFR-I” or “30 kDa TNF inhibitor”) and a soluble TNF receptor type II (also known as “sTNFR-II” or “40 kDa TNF inhibitor”), collectively termed “sTNFRs”, as well as modified forms thereof (e.g., fragments, functional derivatives and variants). EP 393 438 and EP 422 339 also disclose methods for isolating the genes responsible for coding the inhibitors, cloning the gene in suitable vectors and cell types and expressing the gene to produce the inhibitors. Additionally, polyvalent forms (i.e., molecules comprising more than one active moiety) of sTNFR-I and sTNFR-II have also been disclosed. In one embodiment, the polyvalent form may be constructed by chemically coupling at least one TNF inhibitor and another moiety with any clinically acceptable linker, for example polyethylene glycol (WO 92/16221 and WO 95/34326), by a peptide linker (Neve et al. (1996), Cytokine, 8(5):365-370, by chemically coupling to biotin and then binding to avidin (WO 91/03553) and, finally, by combining chimeric antibody molecules (U.S. Pat. No. 5,116,964, WO 89/09622, WO 91/16437 and EP 315062. Anti-TNF antibodies include the MAK 195F Fab antibody (Holler et al. (1993), 1st International Symposium on Cytokines in Bone Marrow Transplantation, 147); CDP 571 anti-TNF monoclonal antibody (Rankin et al. (1995), British Journal of Rheumatology, 34:334-342); BAY X 1351 murine anti-tumor necrosis factor monoclonal antibody (Kieft et al. (1995), 7th European Congress of Clinical Microbiology and Infectious Diseases, page 9); CenTNF cA2 anti-TNF monoclonal antibody (Elliott et al. (1994), Lancet, 344:1125-1127 and Elliott et al. (1994), Lancet, 344:1105-1110).

The IL-17RA-IL-17RB antagonists described herein may be used in combination with all forms of IL-1 inhibitors, such as but not limited to, kiniret (for example ANAKINRA®) (pretreatment, post-treatment, or concurrent treatment). Interleukin-1 receptor antagonist (IL-1ra) is a human protein that acts as a natural inhibitor of interleukin-1. Interleukin-1 receptor antagonists, as well as the methods of making and methods of using thereof, are described in U.S. Pat. No. 5,075,222; WO 91/08285; WO 91/17184; AU 9173636; WO 92/16221; WO 93/21946; WO 94/06457; WO 94/21275; FR 2706772; WO 94/21235; DE 4219626; WO 94/20517; WO 96/22793 and WO 97/28828.

The proteins include glycosylated as well as non-glycosylated IL-1 receptor antagonists. Specifically, three forms of IL-1ra (IL-1raα, IL-1raβ and IL-1 rax), each being encoded by the same DNA coding sequence and variants thereof, are disclosed and described in U.S. Pat. No. 5,075,222. Methods for producing IL-1 inhibitors, particularly IL-1 ras, are also disclosed in the U.S. Pat. No. 5,075,222. An additional class of interleukin-1 inhibitors includes compounds capable of specifically preventing activation of cellular receptors to IL-1. Such compounds include IL-1 binding proteins, such as soluble receptors and monoclonal antibodies. Such compounds also include monoclonal antibodies to the receptors. A further class of interleukin-1 inhibitors includes compounds and proteins that block in vivo synthesis and/or extracellular release of IL-1. Such compounds include agents that affect transcription of IL-1 genes or processing of IL-1 preproteins.

The IL-17RA-IL-17RB antagonists described herein may be used in combination with all forms of CD28 inhibitors, such as but not limited to, abatacept (for example ORENCIA®) (pretreatment, post-treatment, or concurrent treatment). The IL-17RA-IL-17RB antagonists may be used in combination with one or more cytokines, lymphokines, hematopoietic factor(s), and/or an anti-inflammatory agent (pretreatment, post-treatment, or concurrent treatment).

Treatment of the diseases and disorders recited herein can include the use of first line drugs for control of pain and/or inflammation in combination (pretreatment, post-treatment, or concurrent treatment) with treatment with one or more of the IL-17RA-IL-17RB antagonists provided herein. These drugs are classified as non-steroidal, anti-inflammatory drugs (NSAIDs). Secondary treatments include corticosteroids, slow acting antirheumatic drugs (SAARDs), or disease modifying (DM) drugs. Information regarding the following compounds can be found in The Merck Manual of Diagnosis and Therapy, Eighteenth Edition, Merck, Sharp & Dohme Research Laboratories, Merck & Co., Rahway, N.J. (2006) and in Pharmaprojects, PJB Publications Ltd.

In a specific embodiment, the present invention is directed to the use of an IL-17RA-IL-17RB antagonist and any of one or more NSAIDs for the treatment of the diseases and disorders recited herein (pretreatment, post-treatment, or concurrent treatment). NSAIDs owe their anti-inflammatory action, at least in part, to the inhibition of prostaglandin synthesis (Goodman and Gilman in “The Pharmacological Basis of Therapeutics,” MacMillan 7th Edition (1985)). NSAIDs can be characterized into at least nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

In another specific embodiment, the present invention is directed to the use of an IL-17RA-IL-17RB antagonist in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate, magnesium salicylate, choline salicylate, diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate, sodium salicylate and sulfasalazine. Structurally related salicylic acid derivatives having similar analgesic and anti-inflammatory properties are also intended to be encompassed by this group.

In an additional specific embodiment, the present invention is directed to the use of IL-17RA-IL-17RB antagonist in combination (pretreatment, post-treatment, or concurrent treatment) with any of one or more propionic acid derivatives, prodrug esters or pharmaceutically acceptable salts thereof. The propionic acid derivatives, prodrug esters, and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, naproxen sodium, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group.

In yet another specific embodiment, the present invention is directed to the use of an IL-17RA-IL-17RB antagonist in combination (pretreatment, post-treatment, or concurrent treatment) with any of one or more acetic acid derivatives, prodrug esters or pharmaceutically acceptable salts thereof. The acetic acid derivatives, prodrug esters, and pharmaceutically acceptable salts thereof comprise: acemetacin, alclofenac, amfenac, bufexamac, cinmetacin, clopirac, delmetacin, diclofenac potassium, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, tolmetin sodium, zidometacin and zomepirac. Structurally related acetic acid derivatives having similar analgesic and anti-inflammatory properties are also intended to be encompassed by this group.

In another specific embodiment, the present invention is directed to the use of an IL-17RA-IL-17RB antagonist in combination (pretreatment, post-treatment, or concurrent treatment) with any of one or more fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefenamic acid, niflumic acid, talniflumate, terofenamate, tolfenamic acid and ufenamate. Structurally related fenamic acid derivatives having similar analgesic and anti-inflammatory properties are also intended to be encompassed by this group.

In an additional specific embodiment, the present invention is directed to the use of an IL-17RA-IL-17RB antagonist in combination (pretreatment, post-treatment, or concurrent treatment) with any of one or more carboxylic acid derivatives, prodrug esters or pharmaceutically acceptable salts thereof. The carboxylic acid derivatives, prodrug esters, and pharmaceutically acceptable salts thereof which can be used comprise: clidanac, diflunisal, flufenisal, inoridine, ketorolac and tinoridine. Structurally related carboxylic acid derivatives having similar analgesic and anti-inflammatory properties are also intended to be encompassed by this group.

In yet another specific embodiment, the present invention is directed to the use of an IL-17RA-IL-17RB antagonist in combination (pretreatment, post-treatment, or concurrent treatment) with any of one or more butyric acid derivatives, prodrug esters or pharmaceutically acceptable salts thereof. The butyric acid derivatives, prodrug esters, and pharmaceutically acceptable salts thereof comprise: bumadizon, butibufen, fenbufen and xenbucin. Structurally related butyric acid derivatives having similar analgesic and anti-inflammatory properties are also intended to be encompassed by this group.

In another specific embodiment, the present invention is directed to the use of an IL-17RA-IL-17RB antagonist in combination (pretreatment, post-treatment, or concurrent treatment) with any of one or more oxicams, prodrug esters, or pharmaceutically acceptable salts thereof. The oxicams, prodrug esters, and pharmaceutically acceptable salts thereof comprise: droxicam, enolicam, isoxicam, piroxicam, sudoxicam, tenoxicam and 4-hydroxyl-1,2-benzothiazine 1,1-dioxide 4-(N-phenyl)-carboxamide. Structurally related oxicams having similar analgesic and anti-inflammatory properties are also intended to be encompassed by this group.

In still another specific embodiment, the present invention is directed to the use of an IL-17RA-IL-17RB antagonist in combination (pretreatment, post-treatment, or concurrent treatment) with any of one or more pyrazoles, prodrug esters, or pharmaceutically acceptable salts thereof. The pyrazoles, prodrug esters, and pharmaceutically acceptable salts thereof which may be used comprise: difenamizole and epirizole. Structurally related pyrazoles having similar analgesic and anti-inflammatory properties are also intended to be encompassed by this group.

In an additional specific embodiment, the present invention is directed to the use of an IL-17RA-IL-17RB antagonist in combination (pretreatment, post-treatment or, concurrent treatment) with any of one or more pyrazolones, prodrug esters, or pharmaceutically acceptable salts thereof. The pyrazolones, prodrug esters and pharmaceutically acceptable salts thereof which may be used comprise: apazone, azapropazone, benzpiperylon, feprazone, mofebutazone, morazone, oxyphenbutazone, phenylbutazone, pipebuzone, propylphenazone, ramifenazone, suxibuzone and thiazolinobutazone. Structurally related pyrazalones having similar analgesic and anti-inflammatory properties are also intended to be encompassed by this group.

In another specific embodiment, the present invention is directed to the use of an IL-17RA-IL-17RB antagonist in combination (pretreatment, post-treatment, or concurrent treatment) with any of one or more of the following NSAIDs: c-acetamidocaproic acid, S-adenosyl-methionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixirn, lefetamine HCl, leflunomide, lofemizole, lotifazole, lysin clonixinate, meseclazone, nabumetone, nictindole, nimesulide, orgotein, orpanoxin, oxaceprol, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, AI77B, AP504, AU8001, BPPC, BW540C, CHINOIN 127, CN100, EB382, EL508, F1044, FK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770. Structurally related NSAIDs having similar analgesic and anti-inflammatory properties to the NSAIDs are also intended to be encompassed by this group.

In still another specific embodiment, the present invention is directed to the use of an IL-17RA-IL-17RB antagonist in combination (pretreatment, post-treatment or concurrent treatment) with any of one or more corticosteroids, prodrug esters or pharmaceutically acceptable salts thereof for the treatment of the diseases and disorders recited herein. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flucinolone acetonide, flunisolide, fluocinonide, fluorocinolone acetonide, fluocortin butyl, fluocortolone, fluocortolone hexanoate, diflucortolone valerate, fluorometholone, fluperolone acetate, fluprednidene acetate, fluprednisolone, flurandenolide, formocortal, halcinonide, halometasone, halopredone acetate, hydro-cortamate, hydrocortisone, hydrocortisone acetate, hydro-cortisone butyrate, hydrocortisone phosphate, hydrocortisone 21-sodium succinate, hydrocortisone tebutate, mazipredone, medrysone, meprednisone, methylprednisolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

In another specific embodiment, the present invention is directed to the use of an IL-17RA-IL-17RB antagonist in combination (pretreatment, post-treatment, or concurrent treatment) with any of one or more slow-acting antirheumatic drugs (SAARDs) or disease modifying antirheumatic drugs (DMARDS), prodrug esters, or pharmaceutically acceptable salts thereof for the treatment of the diseases and disorders recited herein. SAARDs or DMARDS, prodrug esters and pharmaceutically acceptable salts thereof comprise: allocupreide sodium, auranofin, aurothioglucose, aurothioglycanide, azathioprine, brequinar sodium, bucillamine, calcium 3-aurothio-2-propanol-1-sulfonate, chlorambucil, chloroquine, clobuzarit, cuproxoline, cyclo-phosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxychloroquine sulfate, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine. Structurally related SAARDs or DMARDs having similar analgesic and anti-inflammatory properties are also intended to be encompassed by this group.

In another specific embodiment, the present invention is directed to the use of an IL-17RA-IL-17RB antagonist (pretreatment, post-treatment, or concurrent treatment) with any of one or more COX2 inhibitors, prodrug esters or pharmaceutically acceptable salts thereof for the treatment of the diseases and disorders recited herein. Examples of COX2 inhibitors, prodrug esters or pharmaceutically acceptable salts thereof include, for example, celecoxib. Structurally related COX2 inhibitors having similar analgesic and anti-inflammatory properties are also intended to be encompassed by this group. Examples of COX-2 selective inhibitors include but not limited to etoricoxib, valdecoxib, celecoxib, licofelone, lumiracoxib, rofecoxib, and the like.

Treatment of the diseases and disorders recited herein can include the use of first line drugs for control of inflammatory responses such as hyperresponsiveness in the airway of an affected individual in combination (pretreatment, post-treatment, or concurrent treatment) with treatment with one or more of the IL-17RA-IL-17RB antagonists provided herein. Drugs that are frequently used in treatment of such diseases or conditions include beta2-agonists, leukotriene inhibitors, methylxanthines, anti-inflammatory agents, anticholinergic agents, bronchodilators, corticosteroids, and combinations of such agents. Information regarding the following compounds can be found in The Merck Manual of Diagnosis and Therapy, Eighteenth Edition, Merck, Sharp & Dohme Research Laboratories, Merck & Co., Rahway, N.J. (2006) and in Pharmaprojects, PJB Publications Ltd.

In a further specific embodiment, the present invention is directed to the use of an IL-17RA-IL-17RB antagonist (pretreatment, post-treatment, or concurrent treatment) with any of one or more beta-2 agonists, prodrug esters or pharmaceutically acceptable salts thereof for the treatment of the diseases and disorders recited herein. Examples of beta-2 agonists, prodrug esters or pharmaceutically acceptable salts thereof include, for example, albuterol (Accuneb®, Proair HFA®, Proventil® HFA, Ventolin HFA®), metaproterenol (Alupent®, Alupent® Inhalation Solution, Alupent® Syrup), pirbuterol acetate (Maxair Autohaler®), and terbutaline sulfate (Brethair®, Brethine®,). Long-acting beta-2 agonists, some of which are combined with other agents (for example, Advair®, Symbicort®, Serevent®, and Foradil®) are also known, and are useful in combination with the IL-17RA-IL-17RB antagonists.

An additional embodiment of the present invention is directed to the use of an IL-17RA-IL-17RB antagonist (pretreatment, post-treatment, or concurrent treatment) with any of one or more leukotriene inhibitors, prodrug esters or pharmaceutically acceptable salts thereof for the treatment of the diseases and disorders recited herein. Examples of leukotriene inhibitors, prodrug esters or pharmaceutically acceptable salts thereof include, for example, zileuton (Zyflo®), zafirlukast (Accolate®), and montelukast (Singulair®).

In a further specific embodiment, the present invention is directed to the use of an IL-17RA-IL-17RB antagonist (pretreatment, post-treatment, or concurrent treatment) with any of one or more methylxanthines, prodrug esters or pharmaceutically acceptable salts thereof for the treatment of the diseases and disorders recited herein. Examples of methylxanthines, prodrug esters or pharmaceutically acceptable salts thereof include, for example, theophylline (for example, Bronkodyl®, Elixophyllin®, Slo-bid®, Slo-Phyllin®, Theo-24®, Theo-Dur®, Theolair®, Uniphyl®) and aminophylline (for example, Phyllocontin®, Truphylline®).

In another specific embodiment, the present invention is directed to the use of an IL-17RA-IL-17RB antagonist (pretreatment, post-treatment, or concurrent treatment) with any of one or more anti-inflammatory agents, prodrug esters or pharmaceutically acceptable salts thereof for the treatment of the diseases and disorders recited herein. Examples of such anti-inflammatory agents include but are not limited to Cromolyn (Nasalcrom®, Intal®, Opticrom®) and nedocromil (Tilade®).

An additional embodiment of the present invention is directed to the use of an IL-17RA-IL-17RB antagonist (pretreatment, post-treatment, or concurrent treatment) with any of one or more anticholinergic agents, prodrug esters or pharmaceutically acceptable salts thereof for the treatment of the diseases and disorders recited herein. Examples of such anticholinergic agents, prodrug esters or pharmaceutically acceptable salts include but are not limited to ipratropium bromide (Atrovent®) and tiotropium (Spiriva®).

In an additional specific embodiment, the present invention is directed to the use of an IL-17RA-IL-17RB antagonist (pretreatment, post-treatment, or concurrent treatment) with any of one or more corticosteroids, prodrug esters or pharmaceutically acceptable salts thereof for the treatment of the diseases and disorders recited herein. Examples of inhaled corticosteroids include beclomethasone dipropionate (Beclovent®, Beconase®, Vancenase®, and Vanceril®), triamcinolone acetonide (Azmacort®, Nasacort®, Tri-Nasal®), and flunisolide (Aerobid®, Nasalide®). Examples of other corticosteroids usefull I the present invention include predisone (Prednisone Intensol®, Sterapred®) and prednisolone (Orapred®, Pediapred®, Prelone®).

Yet another specific embodiment of the present invention is directed to the use of an IL-17RA-IL-17RB antagonist (pretreatment, post-treatment, or concurrent treatment) with any of one or more inhaled beta-2 agonists, prodrug esters or pharmaceutically acceptable salts thereof for the treatment of the diseases and disorders recited herein. Examples of corticosteroids, prodrug esters or pharmaceutically acceptable salts thereof include, for example, albuterol (Ventolin®, Proventil®), metaproterenol (Alupent®), pirbuterol acetate (Maxair®), terbutaline (Brethine®, Brethaire®), isoetharine (Bronkosol®) and levalbuterol (Xopenex®).

In a further specific embodiment, the present invention is directed to the use of an IL-17RA-IL-17RB antagonist (pretreatment, post-treatment, or concurrent treatment) with any of one or more bronchodilators (or anticholinergic agents), prodrug esters or pharmaceutically acceptable salts thereof for the treatment of the diseases and disorders recited herein. Examples of bronchodilators include ipratropium (Atrovent®) and tiotropium (Spiriva®).

Treatment of the diseases and disorders recited herein can include the use of first line drugs for treatment or control of an infectious disease in combination (pretreatment, post-treatment, or concurrent treatment) with treatment with one or more of the IL-17RA-IL-17RB antagonists provided herein. Drugs that are frequently used in treatment of such diseases or conditions include antibiotics, antimicrobials, antiviral agents, and combinations thereof. Information regarding the following compounds can be found in The Merck Manual of Diagnosis and Therapy, Eighteenth Edition, Merck, Sharp & Dohme Research Laboratories, Merck & Co., Rahway, N.J. (2006) and in Pharmaprojects, PJB Publications Ltd.

In still another specific embodiment, the present invention is directed to the use of an IL-17RA-IL-17RB antagonist in combination (pretreatment, post-treatment, or concurrent treatment) with any of one or more antimicrobials, prodrug esters or pharmaceutically acceptable salts thereof for the treatment of the diseases and disorders recited herein. Antimicrobials include, for example, the broad classes of penicillins, cephalosporins and other beta-lactams, aminoglycosides, azoles, quinolones, macrolides, rifamycins, tetracyclines, sulfonamides, lincosamides and polymyxins. The penicillins include, but are not limited to penicillin G, penicillin V, methicillin, nafcillin, oxacillin, cloxacillin, dicloxacillin, floxacillin, ampicillin, ampicillin/sulbactam, amoxicillin, amoxicillin/clavulanate, hetacillin, cyclacillin, bacampicillin, carbenicillin, carbenicillin indanyl, ticarcillin, ticarcillin/clavulanate, azlocillin, mezlocillin, peperacillin, and mecillinam. The cephalosporins and other beta-lactams include, but are not limited to cephalothin, cephapirin, cephalexin, cephradine, cefazolin, cefadroxil, cefaclor, cefamandole, cefotetan, cefoxitin, ceruroxime, cefonicid, ceforadine, cefixime, cefotaxime, moxalactam, ceftizoxime, cetriaxone, cephoperazone, ceftazidime, imipenem and aztreonam. The aminoglycosides include, but are not limited to streptomycin, gentamicin, tobramycin, amikacin, netilmicin, kanamycin and neomycin. The azoles include, but are not limited to fluconazole. The quinolones include, but are not limited to nalidixic acid, norfloxacin, enoxacin, ciprofloxacin, ofloxacin, sparfloxacin and temafloxacin. The macrolides include, but are not limited to erythomycin, spiramycin and azithromycin. The rifamycins include, but are not limited to rifampin. The tetracyclines include, but are not limited to spicycline, chlortetracycline, clomocycline, demeclocycline, deoxycycline, guamecycline, lymecycline, meclocycline, methacycline, minocycline, oxytetracycline, penimepicycline, pipacycline, rolitetracycline, sancycline, senociclin and tetracycline. The sulfonamides include, but are not limited to sulfanilamide, sulfamethoxazole, sulfacetamide, sulfadiazine, sulfisoxazole and co-trimoxazole (trimethoprim/sulfamethoxazole). The lincosamides include, but are not limited to clindamycin and lincomycin. The polymyxins (polypeptides) include, but are not limited to polymyxin B and colistin.

### 4.0 Screening Assays

Additional embodiments include methods of screening for antagonists of the IL-17RA-IL-17RB heteromeric receptor complex. Screening assay formats that are known in the art and are adaptable to identifying antagonists of the IL-17RA-IL-17RB heteromeric receptor complex are contemplated. For example: a method of screening for an antagonist of an IL-17RA-IL-17RB heteromeric receptor complex, comprising providing an IL-17RA and an IL-17RB in an IL-17RA-IL-17RB heteromeric receptor complex; exposing a candidate agent to said receptor complex; and determining the amount of receptor complex formation relative to not having been exposed to the candidate agent. The step of exposing a candidate agent to the receptor complex may be before, during, or after IL-17RA and IL-17RB form an IL-17RA-IL-17RB heteromeric receptor complex.

Additional embodiments include a method of screening for an antagonist of IL-17RA-IL-17RB heteromeric receptor complex activation, comprising providing an IL-17RA and an IL-17RB in an IL-17RA-IL-17RB heteromeric receptor complex; exposing a candidate agent to said receptor complex; adding one or more IL-17 ligands; and determining the amount of IL-17RA-IL-17RB heteromeric receptor complex activation relative to not having been exposed to the candidate agent. Candidate agents that decrease IL-17RA-IL-17RB heteromeric receptor complex activation in the presence of one or more IL-17 ligands, as measured by a biologically relevant readout (see below), are considered positive. The IL-17 ligand may be IL-17A, IL-17F, IL-25 or any other IL-17 ligand that binds and activates IL-17RA, IL-17RB, or the IL-17RA-IL-17RB heteromeric receptor complex. Activation is defined elsewhere in the specification. Relevant biological readouts include IL-5, IL-6, IL-8, IL-13, CXCL1, CXCL2, GM-CSF, G-CSF, M-CSF, IL-1β, TNFα, RANK-L, LIF, PGE2, IL-12, MMP3, MMP9, GROα, NO, as well as any other molecule known in the art to be released from any cells expressing IL-17RA and/or IL-17RB. The step of exposing a candidate agent to the receptor complex may be before, during, or after IL-17RA and IL-17RB form an IL-17RA-IL-17RB heteromeric receptor complex. It is understood that a candidate agent may partially inhibit IL-17RA-IL-17RB heteromeric receptor complex, i.e., less than 100% inhibition. Under certain assay conditions a candidate agent may completely inhibit IL-17RA-IL-17RB heteromeric receptor complex.

In one aspect, the invention provides for cell-based assays to detect the effect of candidate agents on the association of IL-17RA and IL-17RB, the 17RA-IL-17RB heteromeric receptor complex, as well as activation of the 17RA-IL-17RB heteromeric receptor complex. Thus the invention provides for the addition of candidate agents to cells to screen for 17RA-IL-17RB heteromeric receptor complex antagonists.

By “candidate agent” or “candidate drug” as used herein describes any molecule, such as but not limited to peptides, fusion proteins of peptides (e.g., peptides that bind IL-17RA, IL-17RB, or the 17RA-IL-17RB heteromeric receptor complex that are covalently or non-covalently bound to other proteins, such as fragments of antibodies or protein-based scaffolds known in the art), proteins, antibodies, small organic molecules including known drugs and drug candidates, polysaccharides, fatty acids, vaccines, nucleic acids, etc. that can be screened for activity as outlined herein.

Candidate agents encompass numerous chemical classes. In one embodiment, the candidate agent is an organic molecule, preferably small organic compounds having a molecular weight of more than 100 and less than about 2,500 daltons. Included are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In alternative embodiments, the candidate bioactive agents may be proteins or fragments of proteins. Thus, for example, cellular extracts containing proteins, or random or directed digests of proteinaceous cellular extracts, may be used. In this way libraries of procaryotic and eucaryotic proteins may be made for screening in the systems described herein. Included in this embodiment are libraries of bacterial, fungal, viral, and mammalian proteins, including human proteins.

In some embodiments, the candidate agents are peptides. In this embodiment, it can be useful to use peptide constructs that include a presentation structure. By “presentation structure” or grammatical equivalents herein is meant a sequence, which, when fused to candidate bioactive agents, causes the candidate agents to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures. Particular presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc. See U.S. Pat. No. 6,153,380, incorporated by reference.

Of particular use in screening assays are phage display libraries; see e.g., U.S. Pat. Nos. 5,223,409; 5,403,484; 5,571,698; and 5,837,500, all of which are expressly incorporated by reference in their entirety for phage display methods and constructs. In general, phage display libraries can utilize synthetic protein (e.g. peptide) inserts, or can utilize genomic, cDNA, etc. digests.

Depending on the assay and desired outcome, a wide variety of cell types may be used, including eukaryotic and prokaryotic cells, with mammalian cells, and human cells, finding particular use in the invention. In one embodiment, the cells may be genetically engineered, for example they may contain exogenous nucleic acids, such as those encoding IL-17RA and IL-17RB. In some instances, the IL-17RA and IL-17RB proteins of the invention are engineered to include labels such as epitope tags, such as but not limited to those for use in immunoprecipitation assays or for other uses.

The candidate agents are added to the cells and allowed to incubate for a suitable period of time. The step of exposing a candidate agent to the receptor complex may be before, during, or after IL-17RA and IL-17RB form an IL-17RA-IL-17RB heteromeric receptor complex. In one embodiment, the association of IL-17RA and IL-17RB is evaluated in the presence and absence of the candidate agents. For example, by using tagged constructs and antibodies, immunoprecipitation experiments can be done. Candidate agents that interfere with IL-17RA and IL-17RB association are then tested for IL-17 ligand family member (such as IL-17A and IL-17F) signaling activity, such as by testing for expression of genes that are activated by IL-17 ligand family member, as mentioned above.

In some embodiments, the IL-17RA and/or IL-17RB proteins are fusion proteins. For example, receptor proteins may be modified in a way to form chimeric molecules comprising an apoprotein (i.e., the protein moiety of a chimeric molecule or complex) fused to another, heterologous polypeptide or amino acid sequence. In one embodiment, such a chimeric molecule comprises a fusion of one or more receptors with a tag polypeptide which provides an epitope to which an anti-tag antibody can selectively bind. The epitope tag is generally placed at the amino- or carboxyl-terminus of the receptor protein. The presence of such epitope-tagged forms of the receptor can be detected using an antibody against the tag polypeptide. Also, provision of the epitope tag enables the receptor polypeptide to be readily purified by affinity purification using an anti-tag antibody or another type of affinity matrix that binds to the epitope tag. These epitope tags can be used for immobilization to a solid support, as outlined herein.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the FLAGG™-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

A variety of expression vectors can be made. The expression vectors may be either self-replicating extrachromosomal vectors or vectors which integrate into a host genome. Generally, these expression vectors include transcriptional and translational regulatory nucleic acid operably linked to the nucleic acid encoding the metalloprotein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

In some embodiments, the inhibition of binding to IL-17RA-IL-17RB heteromeric receptor complex assays are run in vitro. For example, components of the assay mixture (candidate agent, IL-17RA and IL-17RB) are immobilized on a surface, the other components are added (one of which is labeled in some embodiments). For example, IL-17RA or IL-17RB can be attached to a surface, a candidate agent and a labeled IL-17RA and/or IL-17RB is added. After washing, the presence of the label is evaluated. In this embodiment, the IL-17RA and IL-17RB proteins are isolated as is known in the art.

In general, attachment will generally be done as is known in the art, and will depend on the composition of the two materials to be attached. In general, attachment linkers are utilized through the use of functional groups on each component that can then be used for attachment. Functional groups for attachment are amino groups, carboxy groups, oxo groups, hydroxyl groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, incorporated herein by reference). Attachment linkers include, but are not limited to, alkyl groups (including substituted alkyl groups and alkyl groups containing heteroatom moieties), including short alkyl groups, esters, amide, amine, epoxy groups and ethylene glycol and derivatives. Alternatively, fusion partners are used; suitable fusion partners include other immobilization components, such as histidine tags for attachment to surfaces with nickel, functional components for the attachment of linkers and labels, etc., and proteinaceous labels.

In one embodiment, particularly when the candidate agents are immobilized on a solid support, a suitable fusion partner is an autofluorescent protein label. Suitable proteinaceous fluorescent labels also include, but are not limited to, green fluorescent protein (GFP), including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

The insoluble supports may be made of any composition to which the compositions can be bound, is readily separated from soluble material, and is otherwise compatible with the overall method of screening. The surface of such supports may be solid or porous and of any convenient shape. Examples of suitable supports include microtiter plates, arrays, membranes and beads, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica based materials including silicon and modified silicon, carbon, metals, inorganic glasses, plastics, ceramics, and a variety of other polymers. In some embodiments, the solid supports allow optical detection and do not themselves appreciably fluoresce. In addition, as is known the art, the solid support may be coated with any number of materials, including polymers, such as dextrans, acrylamides, gelatins, agarose, etc. Exemplary solid supports include silicon, glass, polystyrene and other plastics and acrylics. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable.

The candidate agents are contacted with the other components of the assay under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away, in the case of solid phase assays. Assay formats are discussed below.

A variety of other reagents may be included in the assays. These include reagents like salts, neutral proteins, e.g. albumin, detergents, etc which may be used to facilitate optimal apoprotein-agent binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

In one embodiment, any of the assays outlined herein can utilize robotic systems for high throughput screening. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which may be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In one embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In one embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In one embodiment, thermocycler and thermoregulating systems are used for stabilizing the temperature of the heat exchangers such as controlled blocks or platforms to provide accurate temperature control of incubating samples from 4° C. to 100° C.

In some embodiments, the instrumentation will include a detector, which may be a wide variety of different detectors, depending on the labels and assay. In one embodiment, useful detectors include a microscope(s) with multiple channels of fluorescence; plate readers to provide fluorescent, ultraviolet and visible spectrophotometric detection with single and dual wavelength endpoint and kinetics capability, fluoroescence resonance energy transfer (FRET), SPR systems, luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

The 17RA-IL-17RB heteromeric receptor complex is the biologically active form of the receptor and has been shown herein to respond to ligand-specific activation by release of proinflammatory mediators. It is known in the art that various disease states, as exemplified herein, are associated with increased physiological levels of IL-17 ligand family members. In one embodiment, the IL-17RA-IL-17RB antigen binding proteins are useful for detecting IL-17RA-IL-17RB heteromeric receptor complexes in biological samples and identification of cells or tissues that express said complex. This would be of considerable value to the research community.

The antigen binding proteins of the invention can be used for diagnostic purposes to detect, diagnose, or monitor diseases and/or conditions associated with IL-17 or the IL-17RA or IL-17RB receptor. The invention provides for the detection of the presence of the IL-17 receptor in a sample using classical immunohistological methods known to those of skill in the art (e.g., Tijssen, 1993, Practice and Theory of Enzyme Immunoassays, vol 15 (Eds R. H. Burdon and P. H. van Knippenberg, Elsevier, Amsterdam); Zola, 1987, Monoclonal Antibodies: A Manual of Techniques, pp. 147-158 (CRC Press, Inc.); Jalkanen et al., 1985, J. Cell. Biol. 101:976-985; Jalkanen et al., 1987, J. Cell Biol. 105:3087-3096). The detection of the IL-17 receptor can be performed in vivo or in vitro.

Diagnostic applications provided herein include use of the antigen binding proteins to detect expression of the IL-17 IL-17RA and IL-17RB proteins and binding of ligand(s) to the IL-17 receptor. Examples of methods useful in the detection of the presence of the IL-17 receptor include immunoassays, such as the enzyme linked immunosorbent assay (ELISA) and the radioimmunoassay (RIA). As outlined above, the use of co-immunoprecipitation is very useful to detect the IL-17RA-IL-17RB heteromeric receptor complex. For diagnostic applications, the antigen binding protein typically may be labeled with a detectable labeling group as defined herein.

One aspect of the invention provides for identifying a cell or cells that express the IL-17RA-IL-17RB heteromeric receptor complex. In a specific embodiment, the antigen binding protein is labeled with a labeling group and the binding of the labeled antigen binding protein to the IL-17 receptor is detected. In a further specific embodiment, the binding of the antigen binding protein to the IL-17 receptor detected in vivo. In a further specific embodiment, the antigen binding protein-IL-17 receptor is isolated and measured using techniques known in the art. See, for example, Harlow and Lane, 1988, Antibodies: A Laboratory Manual, New York: Cold Spring Harbor (ed. 1991 and periodic supplements); John E. Coligan, ed., 1993, Current Protocols In Immunology New York: John Wiley & Sons.

### 5.0 Making of IL-17RA-IL-17RB Antagonists

Suitable host cells for expression of IL-17RA-IL-17RB antagonists include prokaryotes, yeast, or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, New York, (1985). Cell-free translation systems could also be employed to produce LDCAM polypeptides using RNAs derived from DNA constructs disclosed herein.

Prokaryotes include gram negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, an IL-17RA-IL-17RB antagonist may include an N-terminal methionine residue to facilitate expression of the recombinant polypeptide in the prokaryotic host cell. The N-terminal Met may be cleaved from the expressed recombinant IL-17RA-IL-17RB antagonist.

IL-17RA-IL-17RB antagonists may be expressed in yeast host cells, preferably from the Saccharomyces genus (e.g., S. cerevisiae). Other genera of yeast, such as Pichia, K. lactis or Kluyveromyces, may also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EPA-73,657 or in et. al., Gene, 107:285-195 (1991); and van den Berg et. al., Bio/Technology, 8:135-139 (1990). Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli may be constructed by inserting DNA sequences from pBR322 for selection and replication in E. coli (Amp^{r} gene and origin of replication) into the above-described yeast vectors.

The yeast α-factor leader sequence may be employed to direct secretion of the IL-17RA-IL-17RB antagonist. The α-factor leader sequence is often inserted between the promoter sequence and the structural gene sequence. See, e.g., Kurjan et al., Cell 30:933, 1982; Bitter et al., Proc. Natl. Acad. Sci. USA 81:5330, 1984; U.S. Pat. No. 4,546,082; and EP 324,274. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 μg/ml adenine and 20 μg/ml uracil. Yeast host cells transformed by vectors containing ADH2 promoter sequence may be grown for inducing expression in a “rich” medium. An example of a rich medium is one consisting of 1% yeast extract, 2% peptone, and 1% glucose supplemented with 80 μg/ml adenine and 80 pg/ml uracil. Derepression of the ADH2 promoter occurs when glucose is exhausted from the medium.

Mammalian or insect host cell culture systems could also be employed to express recombinant IL-17RA-IL-17RB antagonists. Baculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line derived from the African green monkey kidney cell line CVI (ATCC CCL 70) as described by McMahan et al. (EMBO J. 10: 2821, 1991).

Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide other genetic elements for expression of a structural gene sequence in a mammalian host cell. Viral early and late promoters are particularly useful because both are easily obtained from a viral genome as a fragment which may also contain a viral origin of replication (Fiers et al., Nature 273:113, 1978). Smaller or larger SV40 fragments may also be used, provided the approximately 250 by sequence extending from the Hind III site toward the Bgl I site located in the SV40 viral origin of replication site is included.

Exemplary expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984 has been deposited as ATCC 39890. Additional useful mammalian expression vectors are described in EP-A-0367566, and in U.S. patent application Ser. No. 07/701,415, filed May 16, 1991, incorporated by reference herein. The vectors may be derived from retroviruses. In place of the native signal sequence, and in addition to an initiator methionine, a heterologous signal sequence may be added, such as the signal sequence for IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type II IL-1 receptor signal peptide described in EP 460,846.

IL-17RA-IL-17RB antagonists, as an isolated, purified or homogeneous protein according to the invention, may be produced by recombinant expression systems as described above or purified from naturally occurring cells.

One process for producing IL-17RA-IL-17RB antagonists comprises culturing a host cell transformed with an expression vector comprising a DNA sequence that encodes at least one IL-17RA-IL-17RB antagonist under conditions sufficient to promote expression of said IL-17RA-IL-17RB antagonist. IL-17RA-IL-17RB antagonist is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. For example, when expression systems that secrete the recombinant protein are employed, the culture medium first may be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, (e.g., silica gel having pendant methyl or other aliphatic groups) can be employed to further purify IL-17RA-IL-17RB antagonists. Some or all of the foregoing purification steps, in various combinations, are well known and can be employed to provide a substantially homogeneous recombinant protein.

It is possible to utilize an affinity column comprising the IL-17RA, or IL-17RB, or both IL-17RA and IL-17RB, or a IL-17RA-IL-17RB heteromeric receptor complex proteins to affinity-purify expressed IL-17RA-IL-17RB antagonists. IL-17RA-IL-17RB antagonists can be removed from an affinity column using conventional techniques, e.g., in a high salt elution buffer and then dialyzed into a lower salt buffer for use or by changing pH or other components depending on the affinity matrix utilized. Alternatively, the affinity column may comprise an antibody that binds IL-17RA-IL-17RB antagonists.

Recombinant protein produced in bacterial culture can be isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

Transformed yeast host cells may be employed to express IL-17RA-IL-17RB antagonists as a secreted polypeptide in order to simplify purification. Secreted recombinant polypeptide from a yeast host cell fermentation can be purified by methods analogous to those disclosed by Urdal et al. 1984, J. Chromatog. 296:171. Urdal et al. describe two sequential, reversed-phase HPLC steps for purification of recombinant human IL-2 on a preparative HPLC column.

All references cited within the body of the instant specification are hereby expressly incorporated by reference in their entirety. The following examples, both actual and prophetic, are provided for the purpose of illustrating specific embodiments or features of the instant invention and do not limit its scope.

### EXAMPLES

Human IL-17RD.HIS, goat anti-hIL-17RA polyclonal antibody, goat anti-hIL-17RB polyclonal antibody, goat anti-hIL-17RC polyclonal antibody, and all ELISA kits were obtained from R & D Systems (Minneapolis, Minn.) and used according to manufacturer's specifications. Murine IL-13 was obtained from Invitrogen Biosource (Carlsbad, Calif.). Murine serum albumen (MSA) was obtained from Sigma-Aldrich (St. Louis, Mo.). Monoclonal antibodies against human and mouse IL-25, IL-17RA and IL-17RB were generated substantially as described by Yao et al. (Yao, et al., 1995, Immunity 3: 811-821; Yao, et al., 1995, J. Immunol. 155:5483-5486; Yao, 1997, Cytokine 9:794-800). cDNAs encoding human and mouse IL-17RA have been described previously (see the three Yao references, supra). Human and mouse IL-17RB encode open reading frames identical to that previously described (Tian, et al., 2000, Oncogene 19(17):2098-2109). cDNAs encoding murine IL-25 have been described previously (Hurst, et al., 2002, J. Immunol. 169(1):443-453.). Murine IL-25 was expressed in E. coli and purified as described (Hurst et al. supra). The extracellular region of human IL-17RA was fused to either poly HIS or human Fc IgG1 (IL-17RA:HIS or IL-17RA:Fc, respectively); the extracellular region of human IL-17RB was fused to either poly HIS (IL-17RB.HIS) or human Fc IgG1 (IL-17RB.Fc) substantially as described in Yao, et al., 1995, Immunity (supra). In some experiments, commercially available murine and human IL-25, IL-17RA Fc, and IL-17RB Fc were used (R & D Systems).

### Example 1

This example demonstrates the requirement for IL-17RB for a response to IL-25 in vivo. IL-17RB−/− mice were generated using methods that are known in the art. Briefly, a gene targeting vector was constructed by replacing genomic sequence containing exon 3 of the murine IL-17RB with a PGKneo cassette. A thymidine kinase cassette (MC-TK) was inserted into the 5′ end of the vector. 129 derived embryonic stem (ES) cells were electroporated with the targeting vector and selected in the presence of G418 and ganciclovir as described (Kolls, J, et al. 1994. Proc. Natl. Acad. Sci. USA. 91:215-219). ES clones carrying a targeted mutation in IL-17RB were identified by a combination of PCR and genomic Southern blot analyses and were injected into Swiss Black blastocysts. Male chimeras were crossed to Swiss Black females to generate mice heterozygous for the IL-17RB mutation which were subsequently intercrossed to generate IL-17RB-deficient mice. These mice were moved to a C57BL/6 background by 5 successive backcrosses to C57BL/6 mice, using Marker-Assisted Accelerated Backcrossing (MAX-BAX^{SM}) technology (Charles River Laboratories, Wilmington, Mass.). Mice that were identified to be 99.5% C57BL/6 were used to establish a breeding colony to produce mice for experimental use.

Control C57BL/6 mice (WT) or IL-17RB−/− mice (KO) were given 50 microL MSA (Sigma-Aldrich, St. Louis Mo.; 10 micrograms/mL) or mouse IL-25 (Amgen; 10 micrograms/mL) intranasally (IN), once per day for four days, substantially as described by Hurst, et al (J. Immunol. 169:443, 2002). On Day 5, bronchoalveolar lavage fluid (BALF) and lung tissue were harvested from the mice and analyzed. Bronchoalveolar lavage (BAL) was performed by intubating mice anesthetized with a 300 microL IP injection of 2.5% Avertin (2-2-2-tribromoethanol, Sigma) and flushing the lungs with two 600 microL volumes of ice-cold Dulbecco's PBS (Gibco). The BAL fluid cells were pelleted by centrifugation at 1000 rpm for 10 minutes and re-suspended with PBS+5% fetal bovine serum (FBS; HyClone; Logan, Utah) for counting and analysis for total leukocyte cellularity as well as for changes in the numbers of various cell types using an ADVIA® 120 hematology machine (a benchtop analyzer for processing and analyzing hematology specimens; Siemens Diagnostics, Tarrytown, N.Y.). The BALF was also tested for IL-5 and IL-13 protein concentrations by ELISA (R&D Systems; limit of detection: IL-5, 31 pg/mL; IL-13, 62 pg/mL).

The levels of mRNAs for various inflammatory mediators in lung tissue were determined by TaqMan® (a rapid, fluorophore-based real-time polymerase chain reaction method) expression using Assays-On-Demand TaqMan® primers (Applied Biosystems, Foster City, Calif.) substantially as described previously (Hartel, C., et al, 1999 Scand.J. Immunol. 49(6):649-654). The TaqMan® analysis was performed on the ABI Prism 7900HT Fast RT-PCR System (Applied Biosystems). The relative expression of each gene to beta-actin, HPRT, or GAPDH gene expression in each treatment group was determined by Sequence Detection System 2.2.3 (Applied Biosystems). Results for two separate experiments are shown in Tables 1-4 below.

TABLE 1
Analysis of BALF cellularity, IL-5 concentrations, and IL-13 concentrations
in IL-17RB KO and WT mice intranasally dosed with IL-25

| Genotype, IN | | | | | | IL-5 | IL-13 |
|---|---|---|---|---|---|---|---|
| treatment | Cellularity | Eosinophils | Neutrophils | Lymphocytes | Macrophages | (pg/mL) | (pg/mL) |
| WT, MSA | 59000 ± 21622 | 220 ± 491 | 520 ± 483 | 980 ± 828 | 57280 ± 21493 | 31 | 63 ± 1 |
| IL-17RB | 48750 ± 24958 | 187 ± 375 | 537 ± 485 | 750 ± 501 | 47275 ± 24110 | 31 | 65 ± 7 |
| KO, MSA | | | | | | | |
| WT, IL-25 | 261000 ± 61482 | 99850 ± 20802 | 67320 ± 20230 | 10020 ± 6376 | 83810 ± 39933 | 206 ± 48 | 296 ± 85 |
| IL-17RB | 90000 ± 15411 | 700 ± 416 | 7470 ± 8945 | 2790 ± 2707 | 79040 ± 13483 | 31 | 64 ± 4 |
| KO, IL-25 | | | | | | | |

N = 5/group; values shown are (average ± SD); samples below the range of detection of the IL-5 ELISA were assigned the value of 31 pg/mL.

TABLE 2
Analysis of IL-5, IL-13, and IL-17RA mRNAs in IL-17RB
KO and WT mice lungs in response to IN IL-25 challenge

| Genotype, IN | | | |
|---|---|---|---|
| treatment | IL-5 | IL-13 | IL-17RA |
| WT, MSA | 0.0001524 ± 0.0002235 | 0.004738 ± 0.005697 | 0.03015 ± 0.01009 |
| IL-17RB KO, | 0.0004190 ± 0.0003338 | 0.006084 ± 0.007098 | 0.02895 ± 0.009634 |
| MSA | | | |
| WT, IL-25 | 0.001011 ± 0.0002767 | 0.02066 ± 0.005046 | N/A |
| IL-17RB KO, | 2.310e−005 ± 1.173e−005 | 3.146e−005 ± 2.068e−005 | N/A |
| IL-25 | | | |

N = 5/group; values shown are (average ± SD); IL-5 and IL-13 values shown are gene expression relative to β-actin (2E−ΔCt) (average ± SD). IL-17RA values shown are gene expression relative to HPRT (2E−ΔCt) (average ± SD). N/A = Not analyzed

The IL-17RB KO mouse with IN IL-25 challenge experiment was repeated in substantially the same manner, with the addition of a mouse interleukin-13 challenge arm (IL-13; Invitrogen Biosource™, Carlsbad, Calif.; dosed once per day for four days, with 50 microL at 10 micrograms/mL); results are shown in Tables 3-4 below.

TABLE 3
Analysis of BALF cellularity in IL-17RB KO and WT
mice in response to IN IL-13 or IL-25 challenge

| Genotype, IN | | | | | |
|---|---|---|---|---|---|
| treatment | Celluarity | Eosinphils | Neutrophils | Lymphocytes | Macrophages |
| WT, MSA | 41625 ± 12479 | 0 | 9500 ± 4143 | 5378 ± 3772 | 26750 ± 7309 |
| IL-17RB KO, | 15875 ± 9578 | 0 | 2625 ± 3351 | 1125 ± 2250 | 12125 ± 4366 |
| MSA | | | | | |
| WT, IL-13 | 49700 ± 38836 | 12600 ± 8459 | 20000 ± 27611 | 6300 ± 1037 | 10800 ± 4192 |
| IL-17RB KO, | 27100 ± 11475 | 8200 ± 3328 | 4200 ± 5586 | 6200 ± 2168 | 8500 ± 5948 |
| IL-13 | | | | | |
| WT, IL-25 | 185400 ± 103332 | 55600 ± 31053 | 70200 ± 74492 | 14600 ± 5355 | 45000 ± 6255 |
| IL-17RB KO, | 23800 ± 4550 | 0 | 4600 ± 3070 | 2500 ± 2318 | 16700 ± 2683 |
| IL-25 | | | | | |

N = 5/group; values shown are (average ± SD)

TABLE 4
Analysis IL-5, IL-13, eotaxin, MCP-1, IL-9, IL-10, IL-17A, and IL-17RA
mRNAs in IL-17RB KO and WT mice lungs in response to IN IL-25 challenge

| Genotype, | | | | |
|---|---|---|---|---|
| IN treatment | IL-13 | IL-5 | Eotaxin | MCP-1 |
| WT, MSA | 1.404e−02 ± 1.159e−02 | 1.111e−03 ± 8.247e−04 | 5.558e−02 ± 4.341e−02 | 1.777e−03 ± 1.056e−03 |
| IL-17RB KO, | 1.546e−02 ± 1.523e−02 | 1.237e−03 ± 1.142e−03 | 5.541e−02 ± 4.541e−02 | 1.621e−03 ± 1.039e−03 |
| MSA | | | | |
| Genotype, | | | | |
| IN treatment | IL-9 | IL-10 | IL-17A | IL-17RA |
| WT, IL-25 | 7.523e−01 ± 2.561e−01 | 5.668e−02 ± 2.070e−02 | 5.795e−01 ± 1.474e−01 | 1.138e−02 ± 3.890e−03 |
| IL-17RB KO, | 8.828e−04 ± 4.888e−04 | 5.335e−04 ± 1.725e−04 | 1.091e−02 ± 3.142e−03 | 1.485e−03 ± 6.347e−04 |
| IL-25 | | | | |
| WT, MSA | 5.098e−04 ± 4.573e−04 | 1.240e−04 ± 5.739e−05 | 2.735e−04 ± 2.080e−04 | 1.208e−02 ± 1.615e−03 |
| IL-17RB KO, | 6.091e−04 ± 5.795e−04 | 1.624e−04 ± 8.018e−05 | 2.551e−04 ± 2.221e−04 | 9.86e−03 ± 1.599e−03 |
| MSA | | | | |
| WT, IL-25 | 4.820e−03 ± 1.108e−03 | 60688e−03 ± 2.235e−03 | 2.310e−03 ± 1.588e−03 | 3.255e−02 ± 1.287e−02 |
| IL-17RB KO, | 1.385e−05 ± 2.399e−05 | 5.868e−04 ± 1.391e−04 | 2.725e−04 ± 4.720e−04 | 1.748e−02 ± 8.050e−03 |
| IL-25 | | | | |

N = 4 lungs from individual mice; values shown are gene expression relative to HPRT (2E−ΔCt) (ave. ± SD)

For lung histopathology experiments, mice were euthanized by CO₂ asphyxiation. Lungs were harvested, fixed in 10% neutral buffered formalin (NBF), processed, sectioned at 6 microm and stained with hematoxylin and eosin (H&E) or periodic acid Schiff (PAS) stain substantially as described (Harkema, J. R., and J. A. Hotchkiss, Am. J. Pathol. 141:307; 1992); the grading scale used in analyzing the tissues sections is shown below for four different categories. The average total inflammation score for each group is reported in Table 5.

TABLE 5
Histological analysis of lung tissue inflammation and goblet cell
hyperplasia in IL-17RB KO and WT mice challenged with IN IL-25

| | IL-17RB KO, | IL-17RB KO, | WT, IL-25 | WT, MSA |
|---|---|---|---|---|
| | IL-25 (N = 5) | MSA (N = 4) | (N = 5) | (N = 5) |
| Goblet cell hyperplasia | 0 | 0 | 2.2 ± 1.3 | 0 |
| Peribronchial inflammation | 0.6 ± 0.5 | 0 | 1.6 ± 0.9 | 0 |
| Bronchopneumonia | 0.8 ± 0.4 | 0 | 1 ± 0 | 0 |
| Pulmonary perivasculitis/vasculitis | 0 | 0 | 0.6 ± 1.3 | 0 |
| Total Score | 2.2 ± 1.1 | 0 | 7.4 ± 1.9 | 0 |

Average score reported ± SD.
Goblet cell hyperplasia (PAS stain)
0 = normal
1 = minimal, goblet cell hyperplasia in large bronchioles
2 = mild, goblet cell hyperplasia in large and medium bronchioles
3 = moderate, goblet cell hyperplasia in large, medium, and some small bronchioles
4 = Marked, goblet cell hyperplasia in all airways
Peribronchial Inflammation
0 = normal
1 = minimal eosinophil/macrophage/lymphocyte cuffs (discontinuous to single layer), no edema
2 = mild eosinophil/macrophage/lymphocyte cuffs (2-5 cells); minimal edema, fibroplasia
3 = moderate eosinophil/macrophage/lymphocyte cuffs (5-10 cells); edema and fibroplasia present
4 = marked eosinophil/macrophage/lymphocyte cuffs (>10 cells); marked edema and fibroplasia
Bronchopneumonia
0 = normal
1 = minimal, focal accumulation of macrophages/neutrophils/eosinophils/MNGCs
2 = mild, focal accumulation of macrophages/neutrophils/eosinophils/MNGCs
3 = moderate, multifocal accumulation of macrophages/neutrophils/eosinophils/MNGCs
4 = marked, multifocal accumulation of macrophages/neutrophils/eosinophils/MNGCs
Pulmonary perivasculitis/vasculitis
0 = normal
1 = minimal eosinophil/lymphocyte/macrophage cuffs (discontinuous to single layer), no intimal infiltration/hyperplasia
2 = mild eosinophil/lymphocyte/macrophage cuffs (2-5 cells); focal eosinophil intimal infiltration and endothelial hyperplasia
3 = moderate eosinophil/lymphocyte/macrophage cuffs (5-10 cells); focally extensive intimal eosinophil infiltration and endothelial hyperplasia with few MNGCs
4 = marked eosinophil/lymphocyte/macrophage cuffs (>10 cells); wall of vessel sometimes effaced and MNGCs prominent; discreet vasculitis present

In wild type C57BL/6 mice, the effects of intranasal administration of IL-25 included (1) increased total BALF leukocyte numbers, including increased numbers of BALF eosinophils, neutrophils, lymphocytes, and macrophages, and increased BALF IL-5 and IL-13 concentrations (Tables 1 and 3), (2) increased lung mRNA levels of IL-5, IL-13, eotaxin, and MCP-1 (Tables 2 and 4), and (3) goblet cell hyperplasia in large and medium airways, and robust perivascular/vascular inflammation involving both arteries and veins, but not alveolar capillaries (Table 5). None of these effects were observed upon intranasal administration of IL-25 to IL-17RB KO mice (Tables 1-5). IL-17RA mRNA is present in IL-17RB KO mice (Tables 2 and 4). These data demonstrate that IL-17RB is required for all of the IL-activities in the lung that have been measured to date.

### Example 2

This example demonstrates the requirement for IL-17RA for a response to IL-25 in vivo. The generation of C57BL/6 IL-17RA−/− mice has been described previously (Ye, P., et al, 2001 J. Exp. Med. 194:519-527). Control C57BL/6 mice (WT) or IL-17RA−/− mice (KO) were treated substantially as described in Example 1 for IL-17RB−/− mice; results are shown in Tables 6 and 7 below.

TABLE 6
Analysis of BALF in IL-17RA KO vs. C57BL/6 WT mice: Cellularity and Protein

| | | | | | | IL-5 | IL-13 |
|---|---|---|---|---|---|---|---|
| | Cellularity | Eosinophils | Neutrophils | Lymphocytes | Macrophages | (pg/mL) | (pg/mL) |
| WT, MSA | 44900 ± 9283 | 5300 ± 5718 | 6400 ± 4022 | 4700 ± 1304 | 28500 ± 5420 | 31 | 76.39 ± 26.88 |
| KO, MSA | 43300 ± 10634 | 1800 ± 1643 | 8400 ± 8422 | 7300 ± 4778 | 25800 ± 4056 | 31 | 94.74 ± 30.19 |
| WT, IL-13 | 191600 ± 54874 | 69100 ± 25297 | 74000 ± 49050 | 22400 ± 10268 | 26100 ± 10096 | 31 | N/A |
| KO, IL-13 | 161900 ± 56720 | 40300 ± 25250 | 62100 ± 19064 | 38400 ± 27869 | 21100 ± 9134 | 31 | N/A |
| WT, IL-25 | 520500 ± 129960 | 206100 ± 36906 | 220000 ± 97248 | 27300 ± 15470 | 67100 ± 21355 | 213.6 ± 115.2 | 2413 ± 1206 |
| KO, IL-25 | 30300 ± 15123 | 0 | 10300 ± 7621 | 4700 ± 3309 | 15300 ± 6048 | 31 | 85.06 ± 12.65 |

N = 5; values shown are (average ± SD). N/A = Not tested. Samples below the range of detection of the IL-5 ELISA were assigned the value of the lower limit of detection which was 31 pg/mL.

TABLE 7
Analysis of Lung Tissue in IL-17RA KO vs. C57BL/6 WT mice: Levels of mRNA

| | IL-5 | IL-13 | Eotaxin | MCP-1 | IL-17RB |
|---|---|---|---|---|---|
| WT, MSA | 4.347e−005 ± 1.198e−005 | 2.105e−005 ± 8.093e−006 | 0.001674 ± 0.0004063 | 0.0001465 ± 2.076e−005 | 0.0001526 ± 6.955e−005 |
| KO, MSA | 7.767e−005 ± 5.119e−005 | 0.0001564 ± 0.0002616 | 0.003062 ± 0.0008565 | 0.0002408 ± 4.665e−005 | 0.0005272 ± 0.0004737 |
| WT, IL-25 | 0.004601 ± 0.002347 | 0.005463 ± 0.003147 | 0.06500 ± 0.01270 | 0.001429 ± 0.0005115 | 0.01030 ± 0.009463 |
| KO, IL-25 | 0.0001166 ± 7.309e−005/− | 9.084e−006 ± 6.040e−006 | 0.002149 ± 0.0009068 | 0.0005612 ± 0.0003233 | 0.001637 ± 0.0006343 |

N = 4; values shown are gene expression relative to β-actin (2E−ΔCt) (average ± SD). Lung tissue from IL-13-treated mice was not analyzed in this experiment

Lung tissues were sectioned, prepared for histological analysis, stained and analyzed substantially as described in Example 1. The average total inflammation score for each group is reported in Table 8.

TABLE 8
Histological analysis of Lung Tissue in IL-17 RA KO vs. WT mice

| | KO, | KO, | WT, | WT, |
|---|---|---|---|---|
| | IL-25 | MSA | IL-25 | MSA |
| Goblet cell | 0.6 ± 0.5 | 0 | 2.8 ± 0.4 | 0 |
| hyperplasia | | | | |
| Peribronchial | 0.8 ± 0.4 | 0.8 ± 0.9 | 3.2 ± .5 | 0 |
| inflammation | | | | |
| Bronchopneumonia | 1.0 ± 0 | 1.0 ± 0 | 1.2 ± 0.5 | 0.6 ± 0.5 |
| Pulmonary | 1.6 ± 0.5 | 1.2 ± 0.5 | 3.4 ± 0.5 | 0.8 ± 0.4 |
| perivasculitis/ | | | | |
| vasculitis | | | | |
| Total Score | 4.0 ± 1 | 3.0 ± 1.4 | 10.6 ± 1.3 | 1.4 ± 0.9 |

N = 5 for all groups except for IL-17RA KO mice treated with MSA, for which N = 4. Average score reported ± SD.

The experiment was repeated in substantially the same manner; results are shown in Tables 9 and 10 below. Histological analysis of the lungs was not performed in this experiment.

TABLE 9
Analysis of BALF in KO vs. WT mice

| | Cellularity | Eosinophils | Neutrophils | Lymphocytes | Macrophages |
|---|---|---|---|---|---|
| WT, MSA | 32200 ± 11066 | 1400 ± 1949 | 3800 ± 2225 | 2700 ± 2490 | 24300 ± 8983 |
| KO, MSA | 44167 ± 4252 | 0 | 7333 ± 763 | 5333 ± 3175 | 31500 ± 1323 |
| WT, IL-13 | 126300 ± 52821 | 60500 ± 39019 | 37300 ± 19110 | 12500 ± 5160 | 16000 ± 4138 |
| KO, IL-13 | 87000 ± 20788 | 19800 ± 9358 | 38000 ± 12525 | 19200 ± 5563 | 10000 ± 3606 |
| WT, IL-25 | 327600 ± 144145 | 129100 ± 63800 | 136900 ± 65040 | 18400 ± 8828 | 43200 ± 16799 |
| KO, IL-25 | 65800 ± 80169 | 1300 ± 1789 | 30100 ± 58729 | 4700 ± 4894 | 29700 ± 17946 |

N = 5; values shown are (average ± SD)

TABLE 10
Analysis of Lung Tissue in KO vs. WT mice: Levels of mRNA

| | IL-13 | IL-5 | IL-17RB |
|---|---|---|---|
| WT, MSA | 3.200e−011 ± 9.360e−012 | 8.280e−005 ± 2.122e−005 | 4.290e−010 ± 2.512e−011 |
| KO, MSA | 5.768e−011 ± 7.292e−011 | 0.0001578 ± 6.837e−005 | 4.373e−010 ± 4.755e−011 |
| WT, IL-13 | 3.953e−011 ± 1.572e−011 | 9.838e−005 ± 5.447e−006 | 3.205e−010 ± 5.943e−011 |
| KO, IL-13 | 2.353e−009 ± 3.857e−009 | 0.0003726 ± 0.0004745 | 6.198e−010 ± 6.295e−010 |
| WT, IL-25 | 6.675e−008 ± 1.662e−008 | 0.008895 ± 0.0008927 | 1.436e−008 ± 4.626e−009 |
| KO, IL-25 | 5.135e−011 ± 2.720e−011 | 0.000196 ± 6.249e−005 | 3.865e−010 ± 7.159e−011 |

| | Eotaxin | MCP-1 |
|---|---|---|
| WT, MSA | 3.377e−009 ± 7.773e−010 | 7.743e−010 ± 1.499e−010 |
| KO, MSA | 2.755e−009 ± 9.058e−010 | 8.060e−010 ± 3.463e−010 |
| WT, IL-13 | 9.333e−009 ± 1.588e−009 | 4.378e−009 ± 1.612e−009 |
| KO, IL-13 | 2.308e−008 ± 7.232e−009 | 9.863e−009 ± 3.469e−009 |
| WT, IL-25 | 1.181e−007 ± 1.734e−008 | 8.005e−009 ± 1.252e−009 |
| KO, IL-25 | 2.690e−009 ± 1.547e−010 | 1.157e−009 ± 4.762e−010 |

N = 4; values shown are gene expression relative to GAPDH (2E−ΔCt) (average ± SD)

In wild type C57BL/6 mice, the effects of IN administration of IL-25 included: (1) increased total BALF leukocyte numbers, increased numbers of BALF eosinophils, neutrophils, lymphocytes, and macrophages, and increased BALF IL-5 and IL-13 concentrations (Tables 1 and 4), (2) goblet cell hyperplasia in large and medium airways, and robust perivascular/vascular inflammation involving both arteries and veins, but not alveolar capillaries (Table 3), and (3) increased lung mRNA levels of IL-5, IL-13, eotaxin, MCP-1, and IL-17RB (Tables 2 and 5). None of these effects were observed upon intranasal administration of IL-25 to IL-17RA KO mice (Tables 1-5), even though IL-17RB mRNA is present in IL-17RA KO mice. These data demonstrate that IL-17RA is required for IL-25 activities in the lung.

### Example 3

This example demonstrates the requirement for IL-17RA and IL-17RB for a response to IL-25 in vitro. The generation of splenocytes has been described previously (Hamilton, et al., 1978, J Clin Invest. 62(6):1303-12). Briefly, individual spleens from C57BL/6 WT, C57BL/6 IL-17RB KO, and C57BL/6 IL-17RA KO mice were removed aseptically and treated with 0.4 mg/mL collagenase D (Roche Applied Science, Indianapolis, Ind.) and 0.1% DNAse-I (Roche Applied Science) in RPMI 1640 (Gibco-Invitrogen, Carlsbad, Calif.) to generate single cell suspensions. Splenocytes were cultured at 2.0×10⁷ cells/ml in complete DMEM media (Gibco-Invitrogen) alone or with the addition of 1 microgram/mL Concanavalin A (Con A; Sigma-Aldrich), or IL-25 (Amgen) at the indicated final concentrations. The cells were cultured for 72 hours at 37° C. in a 5% CO₂ humidified incubator. The supernatants were examined for IL-5 and IL-13 concentrations by ELISA (R&D Systems). The splenocyte assays were repeated twice for each genotype using different litters of IL-17RA KO, IL-17RB KO and WT animals; data from two separate experiments are shown below (Tables 11-14).

TABLE 11
IL-5 and IL-13 production by IL-25 stimulated
IL-17RA KO and WT splenocytes

| IL-25, | IL-17RA KO, | WT, | IL-17RA KO, | WT, |
|---|---|---|---|---|
| ng/mL | IL-5 pg/mL | IL-5 pg/mL | IL-13 pg/mL | IL-13 pg/mL |
| 500.0 | 31 | 1044 ± 423 | 62 | 1581 ± 389 |
| 250.0 | 31 | 1435 ± 580 | 85 ± 43 | 2363 ± 459 |
| 125.0 | 31 | 1481 ± 542 | 119 ± 87 | 2442 ± 103 |
| 62.0 | 32 ± 14 | 1721 ± 641 | 141 ± 87 | 3133 ± 239 |
| 31.0 | 33. ± 19 | 1737 ± 634 | 122 ± 40 | 2520 ± 185 |
| 15.0 | 31 | 1405 ± 624 | 97 ± 55 | 1897 ± 479 |
| 0.1 | 36 ± 6 | 41 ± 16 | 80 ± 38 | 103 ± 82 |
| 0.0 | 31 | 31 | 62 | 62 |

N = 2 individual spleens; values shown are (average ± SD). Samples below the range of detection of the IL-5 ELISA were assigned the value of 31 pg/mL. Samples below the range of detection of the IL-13 ELISA were assigned the value of 62 pg/ml.

TABLE 12
IL-5 and IL-13 production by IL-25 stimulated
WT and IL-17RA KO splenocytes

| IL-25, | IL-17RA KO, | WT, | IL-17RA KO, | WT, |
|---|---|---|---|---|
| ng/mL | IL-5 pg/mL | IL-5 pg/mL | IL-13 pg/mL | IL-13 pg/mL |
| 60 | 31 | 538 ± 200 | 109 ± 7 | 864 ± 335 |
| 20 | 31 | 491 ± 181 | 188 ± 6 | 892 ± 424 |
| 6.66 | 31 | 364 ± 219 | 129 ± 1 | 657 ± 316 |
| 2.22 | 31 | 191 ± 105 | 113 ± 4 | 367 ± 214 |
| 0.7 | 31 | 115 ± 82 | 143 ± 14 | 212 ± 133 |
| 0.2 | 31 | 46 ± 16 | 138 ± 29 | 112 ± 44 |
| 0 | 31 | 31 | 161 ± 13 | 68 ± 27 |
| Con A | 168 ± 64 | 116 ± 28 | 1730 ± 118 | 674 ± 151 |

TABLE 13
IL-5 and IL-13 production by IL-25 stimulated
IL-17RB KO and WT splenocytes

| IL-25, | IL-17RB KO, | WT, | IL-17RB KO, | WT, |
|---|---|---|---|---|
| ng/mL | IL-5 pg/mL | IL-5 pg/mL | IL-13 pg/mL | IL-13 pg/mL |
| 60 | 31 | 358 ± 181 | 62 | 990 ± 470 |
| 20 | 31 | 228 ± 120 | 71 ± 33 | 875 ± 441 |
| 6.66 | 31 | 217 ± 110 | 95 ± 27 | 680 ± 361 |
| 2.22 | 31 | 223 ± 170 | 80 ± 51 | 528 ± 254 |
| 0.7 | 31 | 82 ± 41 | 95 ± 32 | 369 ± 131 |
| 0.2 | 31 | 43 ± 19 | 104 ± 59 | 265 ± 116 |
| 0 | 31 | 31 | 110 ± 30 | 197 ± 66 |
| Con A | 31 | 130 ± 57 | 342 ± 97 | 628 ± 241 |

N = 3 individual spleens; values shown are (average ± SD); Samples below the range of detection of the IL-5 ELISA were assigned the value of 31 pg/mL. Samples below the range of detection of the IL-13 ELISA were assigned the value of 62 pg/ml.

TABLE 14
IL-5 and IL-13 production by IL-25 stimulated
IL-17RB KO and WT splenocytes

| IL-25, | IL-17RB KO, | WT, IL-5 | IL-17RB KO, | WT, IL-13 |
|---|---|---|---|---|
| ng/mL | IL-5 pg/mL | pg/mL | IL-13 pg/mL | pg/mL |
| 60 | 31 | 114 ± 88 | 61 | 250 ± 151 |
| 20 | 31 | 99 ± 62 | 61 | 181 ± 90 |
| 6.66 | 31 | 62 ± 33 | 61 | 140 ± 63 |
| 2.22 | 31 | 36 ± 13 | 67 ± 32 | 108 ± 38 |
| 0.7 | 31 | 32 ± 27 | 61 | 77 ± 22 |
| 0.2 | 31 | 31 | 61 | 61 |
| 0 | 31 | 31 | 61 | 61 |
| Con A | 80 ± 47 | 113 ± 17 | 460 ± 104 | 744 ± 61 |

N = 3 individual spleens; values shown are (average ± SD); Samples below the range of detection of the IL-5 ELISA were assigned the value of 31 pg/mL. Samples below the range of detection of the IL-13 ELISA were assigned the value of 62 pg/ml.

IL-25 stimulation induced production of IL-5 and IL-13 by cultured wild type C57BL/6 splenocytes.

This cytokine production was not induced by IL-25 stimulation of either IL-17RB KO or IL-17RA KO splenocytes (Tables 11-14). Con A, a positive control for splenocyte activation, induced IL-17RB KO splenocytes to produce IL-13 and IL-17RA KO splenocytes to produce IL-5 and IL-13. Con A stimulation did not induce IL-17RB KO splenocytes to produce IL-5 in one experiment, but did induce IL-5 production from IL-17RB KO splenocytes in a second experiment. These in vitro cell culture data provide further support that both IL-17RB and IL-17RA are necessary for IL-25 signaling.

### Example 4

This example characterizes the ability of anti-IL-17RB-M735 and anti-IL-25-M819 antibodies to inhibit an IL-25 response in vitro. Single cell suspensions of splenocytes were prepared using spleens from naive BALB/C mice and diluted to 4×10⁷ cells/mL in complete DMEM media (Gibco-Invitrogen, Carlsbad, Calif.) Cells (100 microL) were added to 96 well plates for a final concentration of 4×10⁶ cells/well with the following conditions:

### Medium Only

10 ng/mL mulL-25 (stimulus control)

10 ng/mL mulL-25+100 ng/mL mulL-17RB.muFc (blocking control)

10 ng/mL mulL-25+463, 154, 51, 17, 5.7, 1.9, 0.64, 0.21, 0.07, 0.023, 0.007, 0.003 ng/ml anti mulL-17RB M735

10 ng/mL mulL-25+1000, 100, 10, 1.0 or 0.1 ng/mL anti mulL-25 M819.

Three separate biological samples, each sample consisting of splenocytes from two naive BALB/c mouse spleens, were tested for each condition listed above, and this was repeated three times in three separate experiments. Cultures were incubated for 72 hours at 37° and 10% CO₂, at which time supernatants were harvested and assayed for IL-5 concentrations by ELISA. Both M735 and M819 inhibited IL-25-induced secretion of IL-5 by mouse splenocytes; the calculated IC50 values for inhibition of IL-25 induced IL-5 production by cultured BALB/c splenocytes for each antibody in 3 separate splenocyte experiments are shown below in Tables 15-16.

TABLE 16
IC50 values for anti-IL-25 M819

| Antibody description | IC50 |
|---|---|
| mAb M819, expt 1 | 0.24 ng/mL |
| mAb M819, expt 2 | 4.2 ng/mL |
| mAb M819, expt 2 | 1.12 ng/mL |

TABLE 15
IC50 values for anti-IL-17RB M735

| Antibody description | IC50 |
|---|---|
| mAb M735, expt 1 | 1.32 ng/mL |
| mAb M735, expt 2 | 0.166 ng/mL |
| mAb M735, expt 3 | 2.15 ng/mL |

IL-25 induced IL-5 production was inhibited by both anti-IL-17RB M735 and anti-IL-25-M819. These data provide further support that IL-17RB is necessary for IL-25 signaling in splenocytes.

### Example 5

This example characterizes the ability of various anti-IL-17RA antibodies to inhibit an IL-25 response in vitro. Single cell suspensions of splenocytes were prepared substantially as described above for Example 4. Cells (100 microL) were added to 96 well plates for a final concentration of 4×10⁶ cells/well with the following conditions:

### Medium Only

10 ng/mL mulL-25 (stimulus control)

10 ng/mL mulL-25+100 ng/mL mulL-17RB.muFc (blocking control)

10 ng/mL mulL-25+either 1000, 100, 10, 1.0 or 0.1 ng/mL anti mulL-17RA monoclonal antibodies.

Three separate biological samples, each sample consisting of splenocytes from two mice, were tested for each condition. Cultures were incubated for 72 hours at 37° and 10% CO₂, at which time supernatants were harvested and assayed for IL-5 concentrations by ELISA. A panel of eight different rat anti-mouse IL-17RA monoclonal antibodies was tested. None of these significantly inhibited IL-25-induced secretion of IL-5 by mouse splenocytes.

In addition to these rat anti-mouse antibodies, one mouse anti-mouse IL-17RA monoclonal antibody, M751, was evaluated twice in this splenocyte assay. M751 inhibited IL-25-induced secretion of IL-5 by mouse splenocytes. The calculated IC50 for anti-mIL-17RA M751 in 2 separate splenocyte experiments is shown below in Table 17. Anti-IL-17RA-M751 was therefore the best anti-IL-17RA inhibitor of IL-25 induced IL-5 production in this splenocyte assay, but it was not as potent an inhibitor compared with anti-IL-17RB-M735 (Table 15).

TABLE 17
IC50 values of anti-IL-17RA-M751

| Antibody description | IC50 |
|---|---|
| mAb M751, expt 1 | 4.03 ng/mL |
| mAb M751, expt 2 | 2.79 ng/mL |

### Example 6

This example demonstrates the inhibition of an IL-25 response in vivo with an antibody against IL-17RA, M751, which inhibited IL-25 activity in an in vitro bioassay (described previously). BALB/c mice were given murine serum albumen (MSA; Sigma, 10 μg/mL) or mouse IL-25 (Amgen, TO; 10 pg/mL) intranasally, once per day for four days. On days 1-4, four hours before intranasal instillation of the MSA or IL-25, mice were injected intraperitoneally with either 200 micrograms of a neutralizing anti-IL-17RA antibody (M751), a neutralizing anti-IL-17A antibody (M210), or an isotype control antibody (Murine Fc; Amgen). On Day 5, bronchoalveolar lavage fluid (BALF) and lung tissue were harvested and analyzed as described previously. Results of two separate experiments are shown in Tables 18-21 below.

TABLE 18
Analysis of BALF cellularity, IL-5, and IL-13 concentrations in BALB/c mice treated with IN
IL-25 in the presence or absence of a blocking antibody to mouse IL-17RA M751 - Experiment 1

| | | | | | | IL-5 | IL-13 |
|---|---|---|---|---|---|---|---|
| Treatment | Cellularity | Eosinophils | Neutrophils | Lymphocytes | Macrophages | (pg/mL,) | (pg/mL,) |
| Murine Fc, | 38400 ± 16850 | 0 | 16000 ± 10828 | 2900 ± 4278 | 19500 ± 3482 | 31 | 62 |
| MSA | | | | | | | |
| M751, MSA | 48000 ± 15320 | 0 | 21300 ± 9991 | 2900 ± 2748 | 23800 ± 7059 | 31 | 62 |
| M210, MSA | 44300 ± 16965 | 0 | 19600 ± 9443 | 3600 ± 3362 | 21100 ± 7805 | 31 | 62 |
| Murine Fc, | 156400 ± 37662 | 18300 ± 7571 | 62600 ± 32190 | 15200 ± 3564 | 60300 ± 14025 | 200 ± 131 | 271 ± 291 |
| IL-25 | | | | | | | |
| M751, IL-25 | 28800 ± 16832 | 0 | 14800 ± 12122 | 600 ± 1342 | 13400 ± 5889 | 31 | 62 |
| M210, IL-25 | 138900 ± 94677 | 25200 ± 15291 | 52100 ± 51574 | 16100 ± 8799 | 45500 ± 22453 | 303 ± 64 | 428 ± 112 |

N = 5; values shown are (average ± SD)

Samples below the range of detection of the IL-5 ELISA were assigned the value of 31 pg/mL. Samples below the range of detection of the IL-13 ELISA were assigned the value of 62 pg/mL.

TABLE 19
Analysis of BALF cellularity, IL-5, and IL-13 concentrations in BALB/c mice treated with IN
IL-25 in the presence or absence of a blocking antibody to mouse IL-17RA M751 - Experiment 2

| | | | | | | IL-5 | IL-13 |
|---|---|---|---|---|---|---|---|
| Treatment | Cellularity | Eosinophils | Neutrophils | Lymphocytes | Macrophages | (pg/mL,) | (pg/mL,) |
| Murine Fc, | 68800 ± 18527 | 1300 ± 1600 | 12900 ± 4789 | 2800 ± 3428 | 51800 ± 14176 | 57 ± 3 | 82 ± 9 |
| MSA | | | | | | | |
| M751, MSA | 40600 ± 26640 | 0 | 14400 ± 18372 | 1500 ± 3000 | 24700 ± 7406 | 96 ± 30 | 117 ± 56 |
| M210, MSA | 37200 ± 15455 | 700 ± 1400 | 7300 ± 1860 | 2000 ± 1643 | 27200 ± 13588 | 83 ± 36 | 119 ± 47 |
| Murine Fc, | 235900 ± 76855 | 23800 ± 12476 | 119500 ± 61007 | 16600 ± 4872 | 76000 ± 11811 | 290 ± 123 | 298 ± 127 |
| IL-25 | | | | | | | |
| M751, IL-25 | 38100 ± 11778 | 0 | 5200 ± 3295 | 3200 ± 2993 | 29700 ± 8755 | 58 ± 17 | 122 ± 40 |
| M210, IL-25 | 99500 ± 41105 | 16700 ± 8121 | 26900 ± 18169 | 10100 ± 3916 | 45800 ± 16067 | 281 ± 104 | 233 ± 87 |

N = 5; values shown are (average ± SD)

TABLE 20
Analysis of IL-13, IL-5, IL-17RB, eotaxin, and MCP-1 mRNAs in Lung Tissue from IN IL-25 challenged
mice in the absence or present of a blocking antibody to mouse IL-17RA M751 - Experiment 1

| | IL-13 | IL-5 | IL-17RB | Exotaxin | MCP-1 |
|---|---|---|---|---|---|
| muFc, MSA, | 1.518e−04 ± 2.387e−05 | 2.893e−04 ± 4.011e−05 | 9.885e−04 ± 6.524e−05 | 9.488e−03 ± 1.219e−03 | 5.430e−03 ± 1.798e−03 |
| M751, MSA, | 1.478e−02 ± 2.354e−02 | 2.286e−03 ± 3.47e−03 | 4.241e−03 ± 5.528e−03 | 3.937e−02 ± 5.416e−02 | 8.210e−03 ± 7.656e−03 |
| M210, MSA, | 1.320e−04 ± 2.501e−05 | 2.895e−04 ± 1.507e−05 | 1.109e−03 ± 1.209e−04 | 8.155e−03 ± 1.668e−04 | 3.178e−03 ± 5.785e−04 |
| muFc, IL-25, | 3.845e−02 ± 1.147e−02 | 6.348e−03 ± 1.959e−03 | 1.570e−02 ± 3.551e−03 | 1.638e−01 ± 1.281e−02 | 1.534e−02 ± 4.643e−03 |
| M751, IL-25, | 7.518e−05 ± 1.225e−05 | 2.165e−04 ± 2.145e−05 | 1.330e−03 ± 1.869e−04 | 6.430e−03 ± 2.422e−03 | 2.983e−03 ± 3.795e−04 |
| M210, IL-25, | 1.100e−01 ± 2.483e−02 | 1.123e−02 ± 3.066e−03 | 2.063e−02 ± 8.663e−03 | 2.108e−01 ± 1.601e−02 | 1.468e−02 ± 1.734e−03 |

N = 4; values shown are gene expression relative to GAPDH (2E−ΔCt) (average ± SD);

TABLE 21
Analysis of IL-13, IL-5, IL-17RB, eotaxin, and MCP-1 mRNAs in Lung Tissue from IN IL-25 challenged
mice in the absence or presence of a blocking antibody to mouse IL-17RA M751 - Experiment 2

| | IL-13 | IL-5 | IL-17RB | Eotaxin | MCP-1 |
|---|---|---|---|---|---|
| muFc, MSA, | 1.420e−04 ± 5.510e−05 | 2.173e−04 ± 3.291e−05 | N/D | 6.673e−03 ± 7.293e−05 | 1.715e−03 ± 2.458e−04 |
| M751, MSA, | 2.205e−04 ± 1.286e−04 | 2.465e−04 ± 5.150e−05 | N/D | 7.040e−03 ± 7.584e−04 | 1.703e−03 ± 4.502e−04 |
| M210, MSA, | 1.073e−04 ± 1.891e−05 | 2.583e−04 ± 4.472e−05 | N/D | 8.983e−03 ± 8.737e−04 | 2.185e−03 ± 4.961e−04 |
| MuFc, IL-25, | 1.749e−01 ± 7.166e−02 | 90993e−03 ± 3.952e−03 | N/D | 1.953e−01 ± 7.327e−03 | 1.509e−02 ± 5.096e−03 |
| M751, IL-25, | 4.120e−04 ± 1.738e−04 | 3.755e−04 ± 1.014e−04 | N/D | 7.440e−03 ± 7.159e−04 | 2.130e−03 ± 2.238e−04 |
| M210, IL-25, | 1.412e−01 ± 4.586e−02 | 7.845e−03 ± 1.617e−03 | N/D | 2.348e−01 ± 5.629e−02 | 6.760e−03 ± 6.823e−04 |

N = 4; values shown are gene expression relative to GAPDH (2E−ΔCt) (average ± SD); N/D = not determined

Treatment with anti-IL-17RA mab M751 inhibited IL-25 induced BALF cellularity, as well as IL-25 induced BALF IL-5 and IL-13 concentrations and lung transcript induction. In contrast, treatment with anti-IL-17A mab M210 did not significantly affect IL-25 induced BALF cellularity (although the data suggest possible effects of this antibody on IL-25 induced BALF neutrophil levels). These data, along with those described previously in IL-17RA KO mice, indicate that IL-17RA is required for IL-25 induced BALF cellularity and increases in IL-5 and IL-13 concentrations. The in vivo effects of IL-25 do not appear to be mediated through IL-17A, with the exception of IL-25 induced neutrophil recruitment, as shown by anti-IL-17A treatment significantly reducing IL-25 induced neutrophil influx into the BALF.

### Example 7

This example illustrates the induction of airway hyperresponsiveness (AHR) by IL-25 and the effects thereon of anti-IL-17RA-M751 and anti-IL-17A-M210. BALB/c mice were given MSA or mouse IL-25 IN daily, over a period of four days, substantially as described previously. On day 5, airway responsiveness (AHR) to methacholine (MCh) challenge was first measured noninvasively in conscious, unrestrained mice with a whole body plethysmograph (Buxco Electronics, Troy, N.Y.). Enhanced pause (PENH) was measured based on the pressure waveform in the plethysmograph box in response to increasing concentrations of MCh challenge, and is reported as the percent change relative to baseline readings performed prior to MChS exposure. The PC200 is the concentration of MCh required to induce a PENH 200% above baseline, and is reported here in Tables 22 and 23 below.

TABLE 22
AHR to MCh challenge of BALB/c mice treated with
IN IL-25 in the presence or absence of a blocking
antibody to mouse IL-17RA or IL-17A

| Treatment | PC200, MCh, mg/mL |
|---|---|
| muFc, MSA | 19.3 ± 5.3 |
| M751, MSA | 20.7 ± 7.6 |
| M210, MSA | 25.8 ± 10.6 |
| muFc, IL-25 | 4 ± 4.7 |
| M751, IL-25 | 15.9 ± 1.5 |
| M210, IL-25, | 2 ± 2.2 |

N = 5/group; values shown are (average ± SD)

TABLE 23
AHR to methacholine challenge of BALB/c mice treated
with IN IL-25 in the presence or absence of a
blocking antibody to mouse IL-17RA M751

| Treatment | PC200, MCh, mg/mL |
|---|---|
| muFc, MSA | 12.5 ± 2.6 |
| M751, MSA | 17.5 ± 3.4 |
| M210, MSA | 21.5 ± 3.4 |
| MuFc, IL-25 | 4.3 ± 0.5 |
| M751, IL-25 | 9.0 ± 1.7 |
| M210, IL-25 | 5.3 ± 1.3 |

N = 4/group; values shown are (average ± SD)

Airway hyperresponsiveness was also measured in anesthetized and mechanically ventilated mice intranasally dosed with IL-25 and treated with antiIL-17RA-M751. BALB/c mice were given MSA or mouse IL-25 IN daily, over a period of four days, substantially as described previously. On day 5, mice were sedated with xylazine hydrochloride (20 mg/kg intraperintoneally), and anesthetized with sodium pentobarbital (100 mg/kg interperintoneally). The trachea was cannulated with a metal needle, and the mouse was connected to a small animal ventilator (flexiVent, SCIREQ: Scientific Respiratory Equipment, Montreal, Canada). Each mouse was ventilated with sinusoidal inspiration and passive expiration with a rate of 150 breaths/minute and amplitude of 10 mL/kg mouse weight. A positive end expiratory pressure (PEEP) of 3.0 cmH2O was established by the connection of the mouse to a water column.

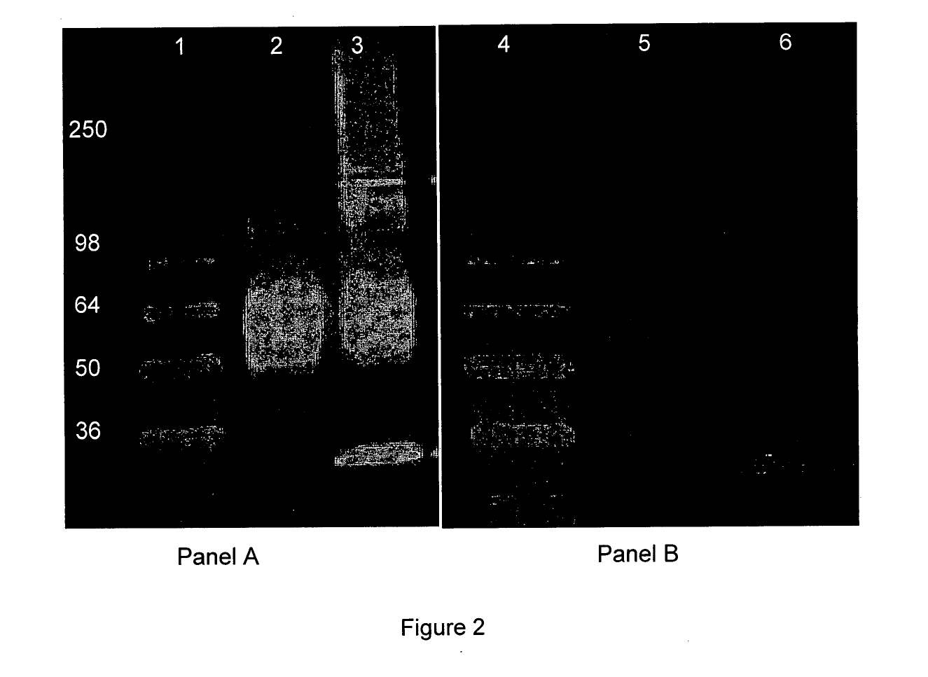

After the mouse was ventilated for one minute, the lungs were expanded twice to total lung capacity (TLC, amplitude pressure of 30 cmH2O). An aerosol of saline or increasing concentrations of acetyl-beta-methylcholine (MCh, Sigma-Aldrich) were delivered to the lung for 15 s followed by 15 s of ventilation. Following aerosol and ventilation, a 2.5 Hz volume driven (VD) oscillation was applied to the airway opening. Each of the 10−2.5 Hz VD oscillations had 0.20 mL amplitude and lasted 1.25 s. Before the next dose of MCh, lungs were expanded twice to TLC. Pressure and volume measurements over time in the respiratory system were recorded by the small animal ventilator, and respiratory system resistance (R) was calculated by fitting the data to the single compartment model of the respiratory system where Pₜᵣ=RV+EV+P_{O} (Pₜᵣ=tracheal pressure, V=volume/time, E=elastance=pressure/volume, V=volume, P_{O}=baseline pressure). Lung resistance measured at different concentrations of MCh are shown in FIG. 2.

These results demonstrated that, in addition to inhibiting IL-25 activity in vitro as well as IL-25 induced BALF cellularity and increased IL-5 and IL-13 concentrations in vivo, M751 inhibited IL-25 induced AHR, indicating that an antibody that binds IL-17RA and inhibits an activity of IL-25 will be useful in treating or ameliorating IL-25 mediated conditions that involve AHR.

### Example 8

This example demonstrates the inhibition of an IL-25 response in vivo with an antibody against IL-17RB (M735) or an antibody against IL-25 (M819), both of which inhibited IL-25 activity in an in vitro bioassay (described above). BALB/c mice were given PBS or mouse IL-25 intranasally, and injected intraperitoneally with 250 micrograms of either a neutralizing mouse anti-mouse IL-17RB antibody (M735), a neutralizing rat anti-mouse IL-25 antibody (M819), an irrelevant control murine IgG1 antibody (mulgGl; Amgen), a murine Fc protein (muFc; Amgen), or whole rat IgG (Pierce, Rockford Ill.). On Day 5, bronchoalveolar lavage fluid (BALF) was harvested and analyzed as described previously. Separate repeat experiments were performed; in the second, BALF IL-5 and IL-13 protein concentrations were not determined. Results are shown in Tables 24-26 below.

TABLE 24
Analysis of BALF cellularity, IL-5 concentrations, and IL-13 concentrations from IN IL-25
challenged mice in the absence or presence of a blocking antibody to IL-17RB (M735)

| | | | | | | IL-5 | IL-13 |
|---|---|---|---|---|---|---|---|
| Treatment | Cellularity | Eosinophils | Neutrophils | Lymphocytes | Macrophages | (pg/mL) | (pg/mL) |
| PBS | 57300 ± 61940 | 600 ± 1200 | 21000 ± 34625 | 3100 ± 6200 | 32600 ± 23484 | 543 ± 155 | 79 ± 21 |
| MuIgG1, IL-25 | 187300 ± 96994 | 28300 ± 13492 | 84100 ± 54232 | 16600 ± 8157 | 58300 ± 23770 | 2680 ± 792 | 754 ± 501 |
| M735, IL-25 | 63500 ± 16646 | 5700 ± 6063 | 17100 ± 5633 | 8600 ± 4127 | 32100 ± 8404 | 909 ± 433 | 82 ± 22 |

N = 5; values shown are (average ± SD)

TABLE 25
Analysis of BALF cellularity, IL-5 concentrations, and IL-13 concentrations
from IN IL-25 challenged mice in the absence or presence of a blocking
antibody to IL-17RB (M735) or a blocking antibody to IL-25 (M819)

| Treatment | Cellularity | Eosinophils | Neutrophils | Lymphocytes | Macrophages |
|---|---|---|---|---|---|
| PBS | 80400 ± 13488 | 0 ± 0 | 17000 ± 7503 | 5400 ± 1364 | 58000 ± 7893 |
| muFc, IL-25 | 277400 ± 38077 | 10400 ± 1363 | 106200 ± 14981 | 29000 ± 2768 | 106200 ± 14981 |
| M735, IL-25 | 50800 ± 8720 | 0 ± 0 | 10400 ± 3501 | 4000 ± 1673 | 50800 ± 8720 |
| rIgG, IL-25, | 144000 ± 43192 | 5400 ± 2293 | 53600 ± 24669 | 20600 ± 4297 | 64400 ± 16157 |
| M819, IL-25 | 99250 ± 29122 | 20600 ± 4261 | 79000 ± 67941 | 23400 ± 9277 | 51260 ± 13668 |

N = 5; values shown are (average ± SD)

TABLE 26
Analysis of BALF cellularity, IL-5 concentrations, and IL-13 concentrations from IN IL-25 challenged mice
in the absence or presence of a blocking antibody to IL-17RB (M735) or a blocking antibody to IL-25 (M819)

| Treat- | | | | | | IL-5 | IL-13 |
|---|---|---|---|---|---|---|---|
| ment | Cellularity | Eosinophils | Neutrophils | Lymphocytes | Macrophages | (pg/mL) | (pg/mL) |
| PBS | 101300 ± 30899 | 8400 ± 1145 | 85900 ± 13106 | 200 ± 200 | 6800 ± 3887 | 68 ± 24 | 90 ± 28 |
| muIgG1, IL-25 | 769400 ± 258902 | 616300 ± 237391 | 50200 ± 10755 | 65800 ± 25649 | 37100 ± 10105 | 230 ± 33 | 598 ± 68 |
| M735, IL-25 | 329800 ± 3774 | 1800 ± 1114 | 23300 ± 2931 | 900 ± 458 | 3800 ± 1007 | 94 ± 16 | 303 ± 125 |
| rIgG, IL-25 | 575700 ± 180123 | 428200 ± 143432 | 48900 ± 10656 | 61300 ± 18219 | 37300 ± 13338 | 230 ± 33 | 598 ± 68 |
| M819, IL-25 | 321400 ± 14445 | 231800 ± 109952 | 30900 ± 11884 | 31400 ± 15182 | 27300 ± 10100 | 121 ± 50 | 476 ± 220 |

N = 5; values shown are (average ± SD)

### Example 9

This example illustrates the induction of airway hypersensitivity reaction (AHR) by IL-25 and the effects thereon of an antibody against IL-17RB (M735) or an antibody against IL-25 (M819). A series of experiments were performed substantially as previously described; AHR was measured noninvasively in conscious, unrestrained mice with a whole body plethysmograph. Results of three separate experiments are shown in tables 27-29 below.

TABLE 27
AHR values from IN IL-25 challenged mice in the absence
or presence of a blocking antibody to anti-IL-17RB (M735)

| Treatment | PC200, MCh, mg/mL |
|---|---|
| PBS | 37.6 ± 15 |
| muIgG1, IL-25 | 5.5 ± 3.2 |
| M735, IL-25, | 16.5 ± 5.7 |

N = 5; values shown are (average ± SD)

TABLE 28
AHR values from IN IL-25 challenged mice in the absence
or presence of a blocking antibody to anti-IL-17RB
(M735) or a blocking antibody to IL-25 (M819)

| Treatment | PC200, MCh, mg/mL |
|---|---|
| PBS | 42 ± 13 |
| muFc, IL-25 | 0.5 ± 0.8 |
| M735, IL-25 | 19.7 ± 6.2 |
| rIgG, IL-25 | 6.1 ± 4.3 |
| M819, IL-25 | 18.4 ± 2.8 |

N = 5; values shown are (average ± SD)

TABLE 29
AHR values from IN IL-25 challenged mice in the absence
or presence of a blocking antibody to anti-IL-17RB
(M735) or a blocking antibody to IL-25 (M819)

| Treatment | PC200, MCh, mg/mL |
|---|---|
| PBS | 29.9 ± 3.3 |
| muIgG1, IL-25 | 6.38 ± 1.2 |
| M735, IL-25 | 16.8 ± 1.6 |
| rIgG, IL-25 | 10.9 ± 1.2 |
| M819, IL-25 | 15.6 ± 1.9 |

These results indicate that IL-25 increases AHR, which effect can be moderated by anti-IL-17RB or anti-IL-25.

### Example 10

This example provides histological confirmation that IL-25 responses in vivo are blocked by treatment with an antibody against IL-17RB (M735), an antibody against IL-25 (M819), or an antibody against IL-17RA (M751). BALB/c mice were given PBS or mouse IL-25 intranasally, and injected intraperitoneally with either 200 micrograms of a neutralizing anti-IL-17RB antibody (M735), 200 micrograms of a neutralizing anti-IL-25 antibody (M819), 200 micrograms of a neutralizing anti-IL-17RA antibody (M751), 200 micrograms of a neutralizing anti-IL17A antibody (M210), or an isotype control antibody substantially as described previously. The mice were euthanized on Day 5 of the study by CO₂ asphyxiation. Lungs were harvested, fixed, processed, sectioned, stained and evaluated as described. A summary of the histopathology results are shown in Table 30 below.

TABLE 30
Histological analysis of lung tissue inflammation and goblet cell
hyperplasia in mice challenged with IN IL-25 and treated with anti-
IL-17RA, anti-IL-17A, anti-IL-25, anti-IL-17RB or control

| | IL-25 + | IL-25 + | IL-25 + | IL-25 + | IL-25 + | MSA + |
|---|---|---|---|---|---|---|
| | M751 | M210 | M819 | M735 | muFc | muFc |
| Goblet cell | 0 | 2.0 ± 1 | 0 | 0.2 ± 0.4 | 2.0 ± 1.4 | 0.0 |
| hyperplasia | | | | | | |
| Peribronchial | 0 | 1.0 ± 1.0 | 0 | 0.0 | 1.8 ± 0.4 | 0.0 |
| inflammation | | | | | | |
| Bronchopneumonia | 1.0 ± 0.7 | 1.8 ± 0.4 | 1.0 ± 1.0 | 0.8 ± 0.8 | 2.0 ± 0 | 1.4 ± 0.5 |
| Pulmonary | 0 | 2.0 ± 0 | 0.4 ± 0.5 | 0.8 ± 0.4 | 1.8 ± 0.4 | 0.4 ± 0.5 |
| perivasculitis/ | | | | | | |
| vasculitis | | | | | | |
| Total Score | 1.0 ± 0.7 | 6.8 ± 1.3 | 1.4 ± 1.1 | 1.8 ± 1.5 | 7.6 ± 2.2 | 1.8 ± 0.8 |

N = 5/group; Average score reported ± SD.

Mice challenged with IL-25 and treated with the isotype control had the most profound lesions and an average score of 7.6±2.2 versus mice challenged with MSA and treated with the isotype control, which had an average score of 1.8±0.8. Treatment of mice with an antibody against IL-17A had essentially no effect on the pulmonary lesions as indicated by the average score of 6.8±1.3. In contrast, treatments with either anti-IL-17RA (score 1.0±0.7), anti-IL-25 (score 1.4±1.1) or anti-IL-17RB (score 1.8±1.5) were all effective at inhibiting IL-25-induced inflammation to the level of background, suggesting that blockade of IL-25 or either one of the proteins involved in the receptor complex were equally effective treatments.

### Example 11

This example demonstrates an association between IL-17RA and IL-17RB. A series of immunoprecipitations was performed using the extracellular domains of human IL-17RA and human IL-17RB fused to the Fc region of human IgG (R&D Systems, Minneapolis, Minn.) or to a polyhistidine tag (Amgen). Fifty microL of Protein G slurry was added to an Eppendorf tube, washed with phosphate buffered saline (PBS), and incubated with 2 microg of IL-17RA.Fc or IL-17RB.Fc protein for one hour at 4° C. with rotation. At the end of this incubation, 2 micrograms of the converse soluble receptor protein (i.e., IL-17RA-HIS was added to IL-17RB:Fc and IL-17RB-HIS was added to IL-17RA:Fc) was added and this final combination was incubated overnight at 4° C. with rotation.

The next morning, the tubes were centrifuged at 12,000 rpm for 1 minute, and the protein G beads were washed with PBS, then RIPA Buffer (Sigma-Aldrich, St. Louis Mo.). The beads were resuspended in 60 microL of 2×Tris-Glycine SDS sample buffer (Invitrogen, Carlsbad Calif.) with 10% beta-mercaptoethanol (Invitrogen, Carlsbad, Calif.) and stored on ice or at −20° C. Samples were analyzed on a 4-20% Tris-Glycine 10 well mini acrylamide gel (Novex®-Invitrogen, Carlsbad Calif.) and transferred to nitrocellulose membrane (Invitrogen, Carlsbad Calif.). Membranes were blocked using Odyssey® Blocking Buffer, a Western Blot blocking buffer optimized for infrared assays (Li-core Biosciences, Lincoln, Nebr.) either at room temperature for 1 hour or overnight at 4° C. with gentle rocking. Membranes were then incubated with primary antibodies diluted 1:1000 to 1:5000 in Odyssey® Blocking Buffer containing 0.1% Tween-20 for 60 minutes at 4° C. with gentle shaking. The membranes were washed 4 times in PBS+0.1% Tween-20, and then incubated in secondary antibody diluted 1:10,000 in Odyssey® Blocking buffer containing 0.1% Tween-20 for 60 minutes at 4° C. with gentle shaking. The membranes were washed 4 times in PBS+0.1% Tween-20 and the proteins were visualized using a Li-Cor® Odyssey® Infrared imaging system. The following antibodies were used:

| Primary Antibodies: | Secondary antibodies: |
|---|---|
| Goat Anti-hIL-17RA affinity | IRDye ® 800CW Donkey anti-Goat |
| purified polyclonal antibody | IgG (H + L), highly adsorbed |
| (R&D Systems, Minneapolis, | (Li-Cor ® Biosciences, Lincoln, NE; |
| MN) | IRDye ® Infrared dyes: US06027709) |
| Goat Anti-hIL-17RB affinity | His•Tag ® Monoclonal Antibody (mouse |
| purified polyclonal antibody | monoclonal antibody (IgG₁) directed |
| (R&D Systems, Minneapolis, | against the His•Tag sequence; Novagen, |
| MN) | EMD Chemicals, Inc., San Diego, CA) |
| Goat Anti-hIL-17RC affinity | |
| purified polyclonal antibody | |
| (R&D Systems, Minneapolis, | |
| MN) | |
| Alexa Fluor ® 680 Rabbit | |
| anti-Mouse IgG (H + L) | |
| (Invitrogen, Carlsbad, CA; | |
| Alexa Fluor680: Berlier J E | |
| et al., J Histochem Cytochem | |
| 51, 1699-712 (2003)) | |

A representative blot is shown in FIG. 2. Over the course of several experiments, IL-17RB.Fc was able to immunoprecipitate IL-17RA.HIS. In this experimental system, IL-17RA.Fc was also able to immunoprecipitate IL-17RC.HIS, demonstrating that this system can reproduce biochemical interactions between proteins that have been demonstrated before in other systems (Toy, D. et al, J I, 2006, 177: 36) Neither IL-17RA.Fc nor IL-17RB.Fc were able to immunoprecipitate IL-17RD.HIS (R&D Systems, Minneapolis, Minn.), suggesting that the IL-17RA and IL-17RB interaction is unique to these proteins and not inherent for all IL-17R family members. This is the first description of a biochemical interaction between IL-17RA and IL-17RB.

### Example 12

The development of fully human monoclonal antibodies directed against human IL-17RA was carried out using Abgenix (now Amgen Fremont Inc.) XenoMouse® technology (U.S. Pat. Nos. 6,114,598; 6,162,963; 6,833,268; 7,049,426; 7,064,244, which are incorporated herein by reference in their entirety; Green et al, 1994, Nature Genetics 7:13-21; Mendez et al., 1997, Nature Genetics 15:146-156; Green and Jakobovitis, 1998, J. Ex. Med. 188:483-495)), as described in U.S. Ser. No. 11/906,094 (incorporated by reference herein) As described therein, fully human anti-IL-17RA antibodies were screened for their ability to inhibit human IL-17A binding to human IL-17RA (and to cynomolgus IL-17RA). A panel of antibodies was identified and selected for further propagation and analysis; the amino acid sequences of the variable heavy and light chains are shown in the sequence listing, and a table summarizing the various sequences is shown below. One antibody, 3.454.1, showed evidence of two versions of the variable light chain.

TABLE 31
Summary of Anti-huIL-17A Antibodies

| Identifier | SEQ ID NO: | Description | SEQ ID NO: | Description |
|---|---|---|---|---|
| 2.133.1 | SEQ ID NO: 1 | AM_{H}1 Vh | SEQ ID NO: 27 | AM_{L}1 Vl |
| 1.98.1 | SEQ ID NO: 2 | AM_{H}2 Vh | SEQ ID NO: 28 | AM_{L}2 Vl |
| 1.235.1 | SEQ ID NO: 3 | AM_{H}3 Vh | SEQ ID NO: 29 | AM_{L}3 Vl |
| 1.185.1 | SEQ ID NO: 4 | AM_{H}4 Vh | SEQ ID NO: 30 | AM_{L}4 Vl |
| 1.166.1 | SEQ ID NO: 5 | AM_{H}5 Vh | SEQ ID NO: 31 | AM_{L}5 Vl |
| 2.98.1 | SEQ ID NO: 6 | AM_{H}6 Vh | SEQ ID NO: 32 | AM_{L}6 Vl |
| 2.67.1 | SEQ ID NO: 7 | AM_{H}7 Vh | SEQ ID NO: 33 | AM_{L}7 Vl |
| 2.46.1 | SEQ ID NO: 8 | AM_{H}8 Vh | SEQ ID NO: 34 | AM_{L}8 Vl |
| 2.225.1 | SEQ ID NO: 9 | AM_{H}9 Vh | SEQ ID NO: 35 | AM_{L}9 Vl |
| 2.159.1 | SEQ ID NO: 10 | AM_{H}10 Vh | SEQ ID NO: 36 | AM_{L}10 Vl |
| 1.124.1 | SEQ ID NO: 11 | AM_{H}11 Vh | SEQ ID NO: 37 | AM_{L}11 Vl |
| 4.357.1 | SEQ ID NO: 12 | AM_{H}12 Vh | SEQ ID NO: 38 | AM_{L}12 Vl |
| 4.180.1 | SEQ ID NO: 13 | AM_{H}13 Vh | SEQ ID NO: 39 | AM_{L}13 Vl |
| 3.1404.1 | SEQ ID NO: 14 | AM_{H}14 Vh | SEQ ID NO: 40 | AM_{L}14 Vl |
| 3.1338.1 | SEQ ID NO: 15 | AM_{H}15 Vh | SEQ ID NO: 41 | AM_{L}15 Vl |
| 4.393.1 | SEQ ID NO: 16 | AM_{H}16 Vh | SEQ ID NO: 42 | AM_{L}16 Vl |
| 4.361.1 | SEQ ID NO: 17 | AM_{H}17 Vh | SEQ ID NO: 43 | AM_{L}17 Vl |
| 4.224.1 | SEQ ID NO: 18 | AM_{H}18 Vh | SEQ ID NO: 44 | AM_{L}18 Vl |
| 4.16.1 | SEQ ID NO: 19 | AM_{H}19 Vh | SEQ ID NO: 45 | AM_{L}19 Vl |
| 3.211.1 | SEQ ID NO: 20 | AM_{H}20 Vh | SEQ ID NO: 46 | AM_{L}20 Vl |
| 3.1545.1 | SEQ ID NO: 21 | AM_{H}21 Vh | SEQ ID NO: 47 | AM_{L}21 Vl |
| 3.381.1.1 | SEQ ID NO: 22 | AM_{H}22 Vh | SEQ ID NO: 48 | AM_{L}22 Vl |
| 3.454.1 | SEQ ID NO: 23 | AM_{H}23 Vh | SEQ ID NO: 49 | AM_{L}23 Vl v 1 |
| 3.454.1.1 | SEQ ID NO: 24 | AM_{H}24 Vh | SEQ ID NO: 50 | AM_{L}23 Vl v 2 |
| 3.891.1 | SEQ ID NO: 25 | AM_{H}25 Vh | SEQ ID NO: 51 | AM_{L}24 Vl |
| 2.23.1 | SEQ ID NO: 26 | AM_{H}26 Vh | SEQ ID NO: 52 | AM_{L}25 Vl |
| | | | SEQ ID NO: 53 | AM_{L}26 Vl |

The antibodies were further characterized with respect to their ability to inhibit IL-17A and/or IL-17F biological activity, and which domains of IL-17RA were important for antibody binding.

IL-17A/IL-17F-Induced Cytokine/Chemokine Secretion Assay

This assays utilizes a human foreskin fibroblast (HFF) cell line. Anti-IL-17RA antibodies are incubated with HFF cells (5000 cells/well in 96 well plate) for 30 minutes at 36° C.; the cultures are then stimulated overnight with either IL-17A (5 ng/ml) alone or IL-17F (20 ng/ml) and TNF-alpha (5 ng/ml). Fibroblast culture supernatants are analyzed by ELISA for the presence of either IL-6 or GRO-alpha. The antibodies were able to inhibit a biological activity of IL-17A and of IL-17F as shown by a reduction in the amount of IL-6 and/or GRO-alpha produced in this assay.

Cross-Competition Assay

Cross-competition studies were performed to determine IL-17RA binding characteristics of certain antibodies, as described in U.S. Ser. No. 11/906,094. A modification of the multiplexed binning method described by Jia, et al. was used (see Jia, et al., J. Immun. Meth., 2004, 288:91-98), employing the Bio-Plex Workstation and software (BioRad, Hercules, Calif.), as well as reagents from Luminex® Corp. (Austin, Tex.). The manufacturers' basic protocols are generally followed. Antibodies were tested in pairwise combinations; if two antibodies cross-competed with each other, they were grouped or “binned” together. Generally speaking, antibodies assigned to different bins bind different parts of IL-17RA and antibodies assigned to the same bin(s) bind similar parts of IL-17RA.

Evaluation of Neutralizing Determinants: Hu/Mu chimeras

Studies were conducted to determine where the various IL-17RA antagonists (in the form of human antibodies) bound to human IL-17RA, using a number of chimeric human/mouse IL-17RA. This method takes advantage of the non-cross reactivity of the various IL-17RA antibodies with mouse IL-17RA. For each chimera, one or two regions of human IL-17RA extracellular domain is/are replaced with the corresponding region(s) of mouse IL-17RA. Six single-region and 8 double-region chimeras were constructed; multiplex analysis using the Bio-Plex Workstation and software (BioRad, Hercules, Calif.) was performed to determine neutralizing determinants on human IL-17RA by analyzing exemplary human IL-17RA mAbs differential binding to chimeric versus wild-type IL-17RA proteins

Evaluation of Neutralizing Determinants: Arginine Scanning

Further studies were conducted using a number of mutant IL-17RA proteins having arginine substitutions at select amino acid residues of human IL-17RA. Arginine scanning is an art-recognized method of evaluating where antibodies, or other proteins, bind to another protein, see for example Nanevicz, T., et al., 1995, J. Biol. Chem., 270:37, 21619-21625 and Zupnick, A., et al., 2006, J. Biol. Chem., 281:29, 20464-20473. In general, the arginine sidechain is positively charged and relatively bulky as compared to other amino acids, which may disrupt antibody binding to a region of the antigen where the mutation is introduced. Arginine scanning is a method that determines if a residue is part of a neutralizing determinant and/or an epitope. Ninety-five amino acids distributed throughout the human IL-17RA extracellular domain were selected for mutation to arginine. The selection was biased towards charged or polar amino acids to maximize the possibility of the residue being on the surface and reduce the likelihood of the mutation resulting in misfolded protein.

Using standard techniques known in the art, sense and anti-sense oligonucleotides containing the mutated residues were designed based on criteria provided by Stratagene Quickchange® protocol kit (Stratagene/Agilent, Santa Clara, Calif.). Mutagenesis of the wild-type (WT) HuIL-17RA-Flag-pHis was performed using a Quickchange® II kit (Stratagene). All chimeric constructs were constructed to encode a FLAG-histidine tag (six histidines) on the carboxy terminus of the extracellular domain to facilitate purification via the poly-His tag, Multiplex analysis using the Bio-Plex Workstation and software (BioRad, Hercules, Calif.) was performed to determine neutralizing determinants on human IL-17RA by analyzing certain human IL-17RA mAbs differential binding to arginine mutants versus wild-type IL-17RA proteins.

Results of these studies are summarized in Table 32 below.

TABLE 32
Summary of the properties of certain IIL-17RA antibodies

| | | Hu/Mu | |
|---|---|---|---|
| Bin | Identifier | Chimera | Arginine Mutants |
| 1 | 4.224.2 | F: 229-319 | S220R, E226R, T228R, S236R, |
| | | | L270R, Q284R |
| 2 | 2.133.1 | B: 75-96 | D152R |
| | | D: 176-197 | |
| 3 | 3.381.1 | C: 128-154 | D152R, D154R, E156R, D184R, |
| | | D: 176-197 | E186R, S198R |
| | 3.1404.1 | D: 176-197 | D152R, D154R, E156R, D184R, |
| | | | E186R, H297R |
| | 4.16.1 | D: 176-197 | D152R, D154R, E156R, D184R, |
| | | | E186R |
| | 3.454.1 | B: 75-96 | E97R, D152R, D154R, E156R, |
| | | C: 128-154 | Q176R, D184R, E186R, S198R, |
| | | D: 176-197 | T235R, H297R |
| 4 | 2.23.1 | F: 229-319 | H138R, K166R, H215R, S220R, |
| | | | E226R, T228R, S236R, E241R, |
| | | | H243R, L270R |
| 5 | 3.1545.1 | D: 176-197 | E113R, S115R, D152R, D154R, |
| | | | E156R, S177R, S198R |
| 6 | 3.211.2 | D: 176-197 | D152R, D154R, E156R, S177R, |
| | | | L270R |

### Example 13

This example describes an IL-25 restimulation assay that is useful for evaluating the effects of IL-17RA-IL-17RB antagonists on a biological activity of IL-25. Human peripheral blood mononuclear cells (PBMC) are isolated from normal donors and stimulated for 24 hours at 5×10⁶ cells/ml in the presence of thymic stromal lymphopoietin (TSLP (Quentmeier et al., Leukemia. 2001 August; 15(8):1286), 100 nanograms/ml; available from R&D Systems, Minneapolis, Minn.). The PBMC are then collected and set up in re-stimulation cultures in the presence of IL-2 (10 nanograms/ml, R&D Systems, Minneapolis, Minn.) and IL-25 (10 nanograms/ml; R&D Systems, Minneapolis, Minn.), in the presence or absence of agents to be tested for inhibitory activity. Restimulation cultures are prepared as a single cell suspension and diluted to 4×10⁷ cells/mL; 100 microL of cells are added to 48 well plates for a final concentration of 4×10⁶ cells/well. After three days, supernatant fluids are harvested and tested for IL-5 by ELISA (R&D Systems, Minneapolis, Minn.). The agents tested include soluble forms of IL-17RB (described previously) and a panel of polyclonal and monoclonal antibodies summarized below:
MAB1771: anti-HuIL-17RA MulgG2b (R&D Systems)
MAB1207: anti-HuIL-17RB MulgG2b (R&D Systems)
AF177: anti-HuIL-17RA Goat Polyclonal IgG (R&D Systems)
Several fully human anti-HuIL-17RA HulgG2 (described in Example 12)

The results are of testing various agents in several different restimulation assays utilizing PBMC from different donors are shown in Table 33 below.

TABLE 33
IL-5 production from TSLP treated human PBMC stimulated
with IL-2 + IL-25 in the presence or absence
of various IL-17RB and IL-17RA inhibitors.

| Inhibitor | [Inhibitor], | [IL-5], | |
|---|---|---|---|
| description | micrograms/mL | pg/mL | % Inhibition |
| None | None | 93.9 ± 9.7 | 0% |
| HuIL-17RB: Fc | 10 | 42.0 ± 10.8 | 55% |
| HuIL-17RB: HIS | 10 | 31.2 ± 11.5 | 67% |
| 3.1404 | 10 | 35.9 ± 5.3 | 62% |
| 3.1404 | 1.0 | 42.9 ± 5.1 | 54% |
| 3.1404 | 0.1 | 83.4 ± 4.6 | 12% |
| None | None | 576 ± 6.8 | 0% |
| HuIL-17RB: Fc | 10 | 72.7 ± 3.8 | 87% |
| MAB1771 | 10 | 437.7 ± 7.8 | 24% |
| MAB1207 | 10 | 499.4 ± 6.3 | 13% |
| AF177 | 10 | 105.8 ± 4.0 | 82% |
| 3.1404 | 10 | 100.5 ± 5.1 | 83% |
| None | None | 191.6 + 4.9 | 0% |
| HuIL-17RB: Fc | 10 | 24.5 ± 4.2 | 88% |
| MAB1771 | 10 | 127.9 ± 3.6 | 33% |
| AF177 | 10 | 26.0 ± 4.0 | 86% |
| 3.1404 | 10 | 19.2 ± 3.4 | 90% |
| 4.16 | 10 | 22.2 ± 3.4 | 88% |
| 3.381 | 10 | 0.0 ± 0.0 | 100% |

A panel of human antibodies that bind IL-17RA was tested, in three separate restimulation assays utilizing different PBMC donor, on different days and with different preparations of antibody. The results are shown in Table 34 below.

TABLE 34
IL-5 production from TSLP treated human PBMC
stimulated with IL-2 + IL-25 in the presence
or absence of various IL-17RA antibodies.

| | | % Inhib., | % Inhib., | % Inhib., |
|---|---|---|---|---|
| Antibody | Bin | Expt. 1 | Expt. 2 | Expt. 3 |
| 3.1404 | 3 | 68% | 76% | nd |
| 4.16 | 3 | 76% | 77% | nd |
| 3.381 | 3 | 90% | 75% | nd |
| 1.243.3 | 1 | 0% | 43% | 0% |
| 1.185.1 | 1 | 0% | 41% | 9% |
| 2.46.1 | 1 | 0% | 6% | 0% |
| 2.67.1 | 1 | 0% | 13% | 3% |
| 2.98.1 | 1 | 0% | 79% | 0% |
| 2.159.2 | 1 | 0% | 86% | 0% |
| 4.224.2 | 1 | 13% | 86% | 53% |
| 2.133.1 | 2 | 0% | 16% | 28% |
| 3.381.1 | 3 | 69% | 82% | 74% |
| 3.454.2 | 3 | 75% | nd | 70% |
| 3.1404.1 | 3 | 77% | 100% | 69% |
| 4.16.1 | 3 | 85% | nd | 100% |
| 4.357.1 | 3 | 0% | nd | 11% |
| 4.361.3 | 3 | 60% | nd | 0% |
| 4.393.1 | 3 | 28% | 72% | 45% |
| 2.23.1 | 4 | 0% | nd | 0% |
| 3.1545.1 | 5 | 0% | 43% | 0% |
| 3.211.2 | 6 | 0% | 47% | 7% |
| 2.225.1 | * | 0% | nd | 0% |
| 4.180.3 | * | 0% | 0% | 0% |

* Results of binning analysis for these antibodies were equivocal.

Substantially similar results were obtained with additional preparations of these antibodies. The results indicate that certain antibodies that bind IL-17RA and inhibit IL-17A also inhibit IL-25.

### Example 14

This example describes a mouse model of asthma. Mice (for example, BALB/c) are sensitized with antigen (for example, ovalbumin [OVA]) by intraperitoneal injection of the antigen in alum or another adjuvant. Several sensitization schemes are known in the art; one scheme is to inject 10 micrograms of OVA in alum three times at one week intervals (i.e., on day −21, day −14 and day −7). The mice are then challenged with antigen either by aerosol exposure (5% OVA) or intranasal administration (0.1 mg OVA). The challenge schedule may be selected from among shorter terms (i.e., daily challenge on days 1, 2 and 3) or longer terms (i.e., weekly challenge for two to three weeks). The endpoints that are measured can include AHR, BAL fluid cell number and composition, in vitro draining lung lymph node cytokine levels, serum IgE levels, and histopathologic evaluation of lung tissue. Other animal models of asthma are known, and include the use of other animals (for example, C57BL/6 mice), sensitization schemes (for example, intranasal inoculation, use of other adjuvants or no adjuvants, etc.) and/or antigens (including peptides such as those derived from OVA or other proteinaceous antigens, cockroach extracts, ragweed extracts or other extracts such as those used in desensitization regimens, etc.). The effects of antibodies to IL-17RA, IL-17RB, IL-17 and IL-25 were evaluated in this model, using groups of mice as shown below.

Female BALB/c mice were immunized IP with OVA in alum on days −21, −14 and −7 and were exposed to an aerosol challenge with OVA in PBS on days 1-3. Mice were injected IV with antibody the day before OVA aerosol challenge (day −1) in Experiments 1 and 2, or IP with antibody the day of the first OVA aerosol challenge (day 1) 30 minutes prior to OVA challenge in Experiment 3, or were injected IP with Dexamethasone (Dex), a positive control, or phosphate buffered saline (PBS), a negative control, 30 minutes before each aerosol exposure to OVA (days 1-3) in Experiments 1 and 3. An age-matched, OVA primed only group was included for comparison. Airway hyperresponsiveness (AHR) to MCh challenge was measured 48 hours after the final OVA challenge. Mice were sacrificed 72 hours after the final OVA challenge and serum, BAL fluid, draining lung lymph nodes, and lungs were collected for analysis. A series of three experiments was performed.

Experiment 1 contained the following treatment groups:
Group 1, primed not challenged mice, n=10
Group 2, PBS, IP, n=10
Group 3, 1 mg/kg Dex, IP, n=10
Group 4, 500 micrograms mIgG1 isotype control ab, IV, n=10
Group 5, 500 micrograms anti-IL-17RB M735 mAb, IV, n=10
Group 6, 500 micrograms chimeric anti-mIL-17RA mAb M751, IV, n=10
Group 7, 500 micrograms rat IgG control ab, IV, n=10
Group 8, 500 micrograms anti-mIL-25 M819, IV, n=10
Group 9, 500 micrograms anti-mIL-17 mAb M210, IV, n=10

Experiment 2 contained the following treatment groups:
Group 1, primed not challenged mice, n=10
Group 2, 500 micrograms mIgG1 isotype control ab, IV, n=10
Group 3, 500 micrograms chimeric anti-mIL-17RA mAb M751, IV, n=10

Experiment 3 contained the following treatment groups:
Group 1, primed not challenged mice, n=10
Group 2, PBS, IP, n=10
Group 3, 1 mg/kg Dex, IP, n=10
Group 4, 500 micrograms mIgG1 isotype control ab, IP, n=10
Group 5, 500 micrograms anti-IL-17RB M735 mAb, IP, n=10
Group 6, 500 micrograms chimeric anti-mIL-17RA mAb M751, IP, n=10
Group 7, 500 micrograms g rat IgG control ab, IP, n=10
Group 8, 500 micrograms anti-mIL-25 M819, IP, n=10
Group 9, 500 micrograms anti-mIL-17 mAb M210, IP, n=10
Group 10, 500 micrograms anti-mIL-17F mAb M850, IP, n=10

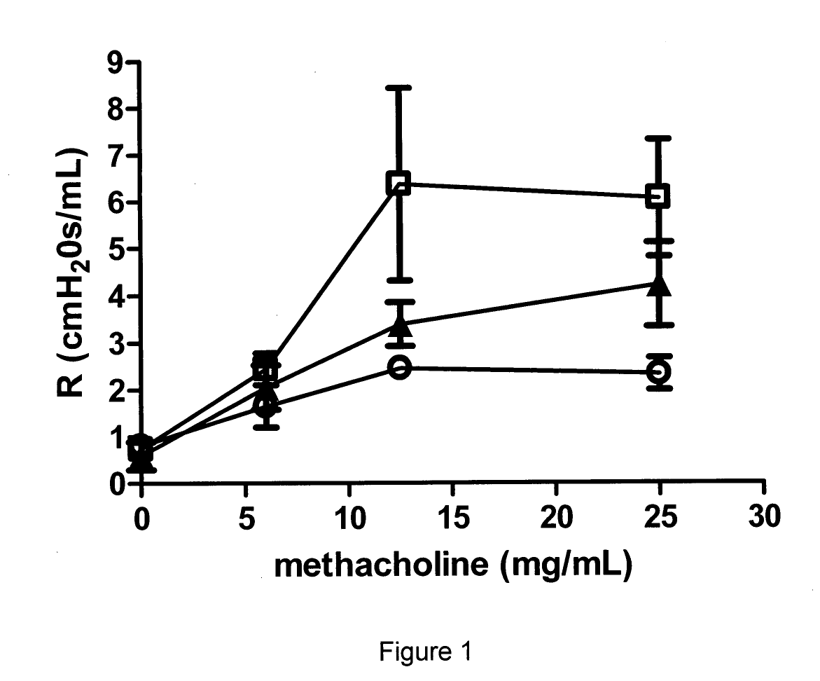

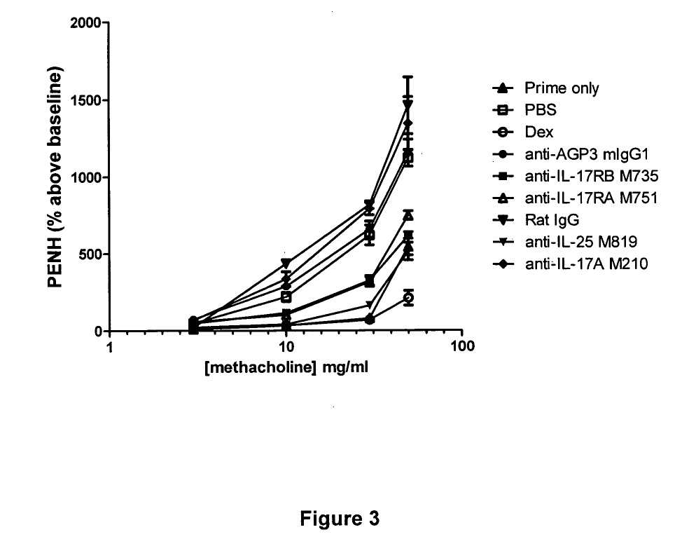

Neutralizing antibodies to IL-17RB, IL-17RA, or IL-25 but not to IL-17A reduced AHR in a mouse OVA asthma model; results are shown in FIGS. 1-3. The mean percent change in PENH relative to baseline is reported for each treatment group±SE from Experiment 1 (FIG. 1). Airway hyperresponsiveness was measured substantially as described previously in Example 7. The degree of bronchoconstriction was expressed as the percentage change in PENH relative to the baseline. Treatment with neutralizing antibodies to IL-17RB, IL-17RA, or IL-25, but not to IL-17A, reduced AHR in response to MCh challenge compared with mice treated with PBS or control antibodies (FIG. 3).

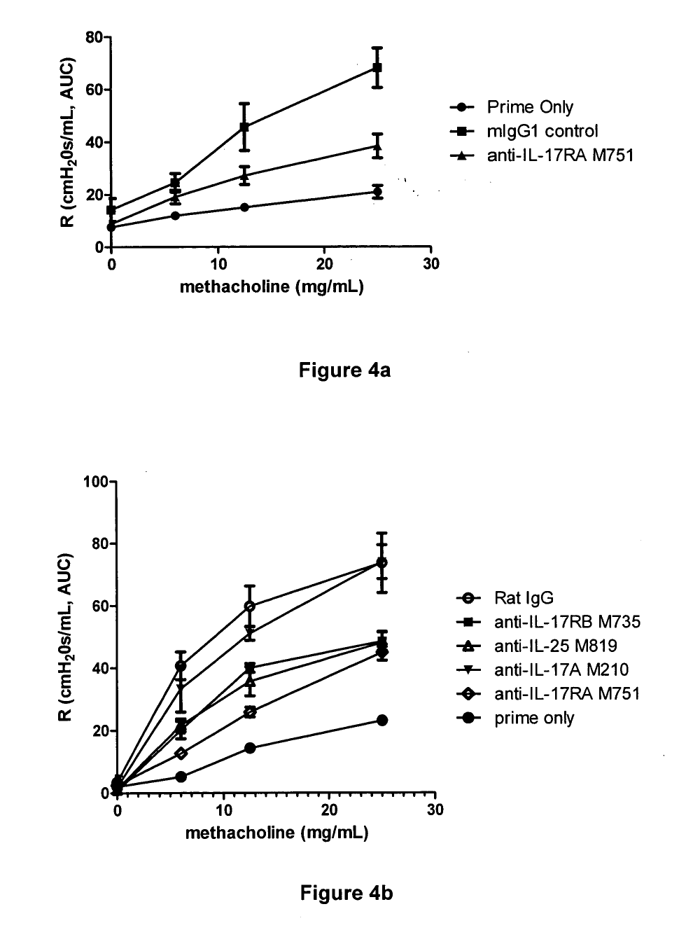

Pulmonary resistance (R_{L}) in response to methacholine challenge was measured in Experiments 2 and 3 in mechanically ventilated mice. Pressure and volume measurements over time in the respiratory system were recorded by the small animal ventilator, and respiratory system resistance (R=cmH₂O/mL) was calculated by fitting the data to the single compartment model of the respiratory system where Pₜᵣ=RV+EV+P_{O} (Pₜᵣ=tracheal pressure, V=volume/time, E=elastance=pressure/volume, V=volume, P_{O}=baseline pressure). Airway resistance (R) area under the curve (AUC) is calculated by taking the sum of all R measurements for each concentration of methacholine for each mouse. In Experiment 2, treatment with a neutralizing antibody to IL-17RA reduced pulmonary resistance in response to methacholine challenge compared with control antibody treated mice (FIG. 4a). In Experiment 3, treatment with neutralizing antibodies to IL-17RB, IL-17RA, or IL-25 but not to IL-17A reduced pulmonary resistance to methacholine challenge compared with control antibody treated mice (FIG. 4b).

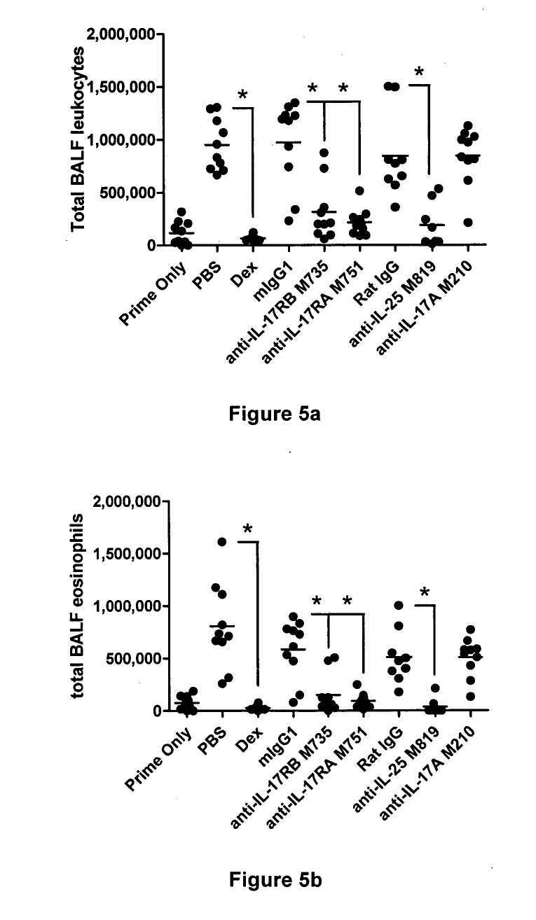

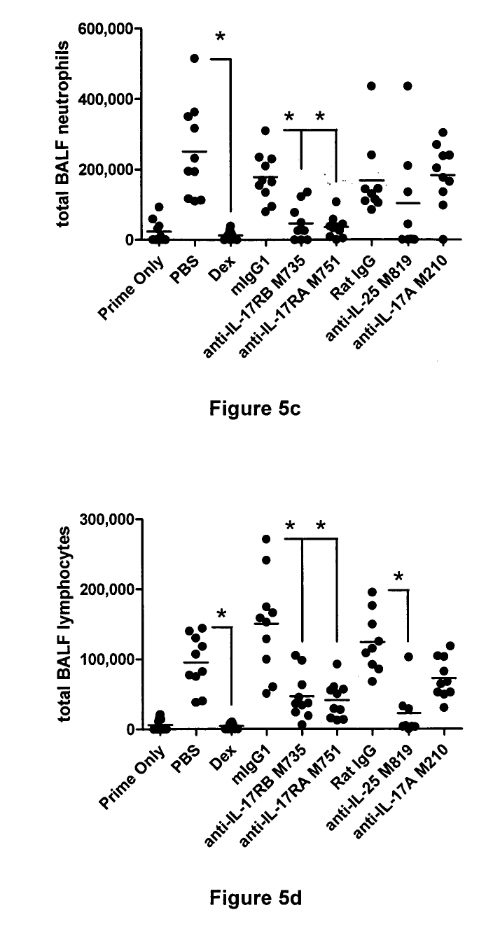

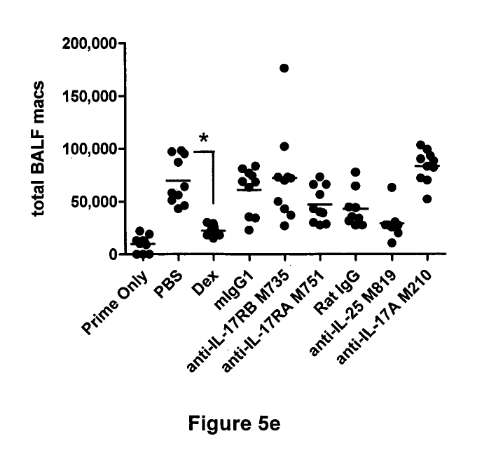

The effects of antibodies on BALF cell number and composition were also determined; results are shown in FIGS. 5-7. In experiment 1, neutralizing antibodies to IL-17RB, IL-17RA, or IL-25 but not to IL-17A significantly reduced BALF total leukocytes (FIG. 5a), eosinophils (FIG. 5b), and lymphocytes (FIG. 5d) compared with the appropriate control antibody treatments in this mouse OVA asthma model. Neutralizing antibodies to IL-17RB or IL-17RA but not to IL-17A significantly reduced BALF total neutrophils (FIG. 5c). The neutralizing antibody to IL-25 reduced BALF total neutrophils but this was not significant (FIG. 5c).

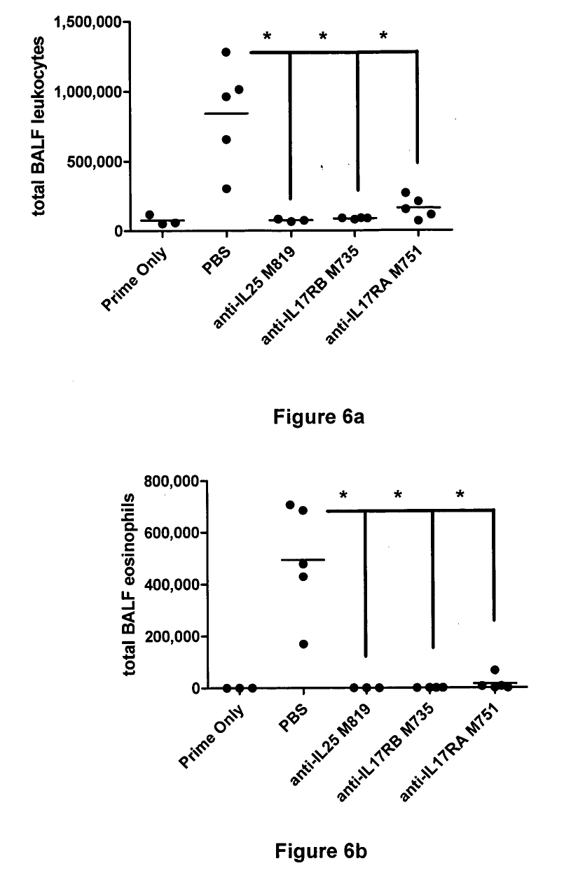

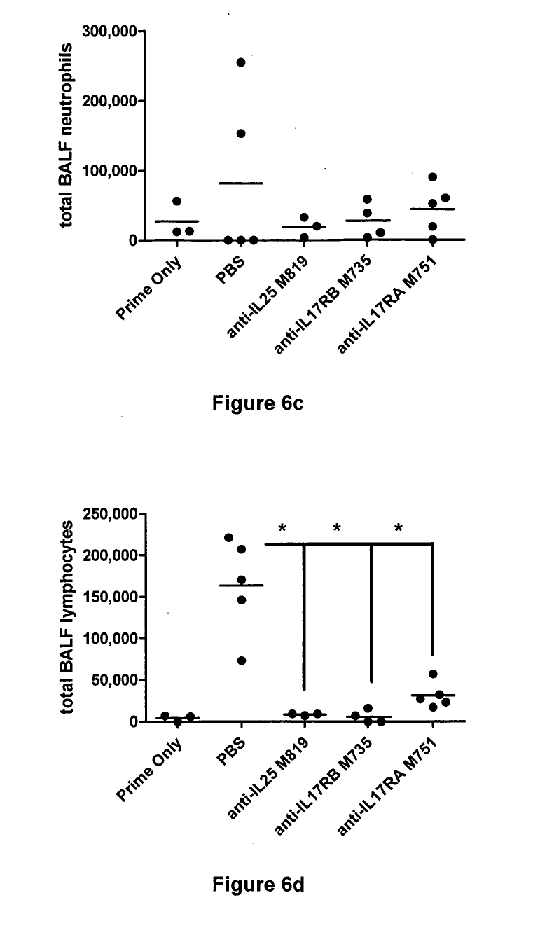

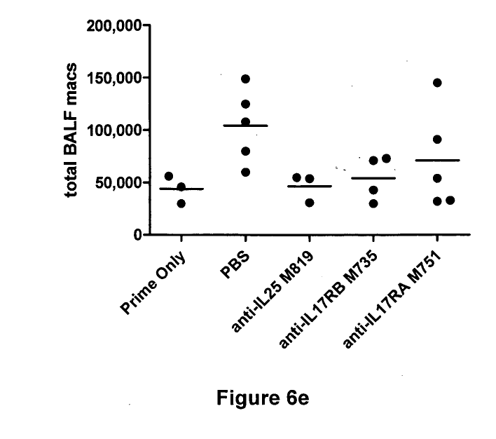

In experiment 2, neutralizing antibodies to IL-25, IL-17RB, and IL-17RA significantly reduced BALF total leukocytes (FIG. 6a), eosinophils (FIG. 6b), and lymphocytes (FIG. 6d) compared with the appropriate control antibody treatments in this mouse OVA asthma model. These antibodies did not have a significant effect on total BALF neutrophil (FIG. 6c) or macrophage (FIG. 6e) numbers.

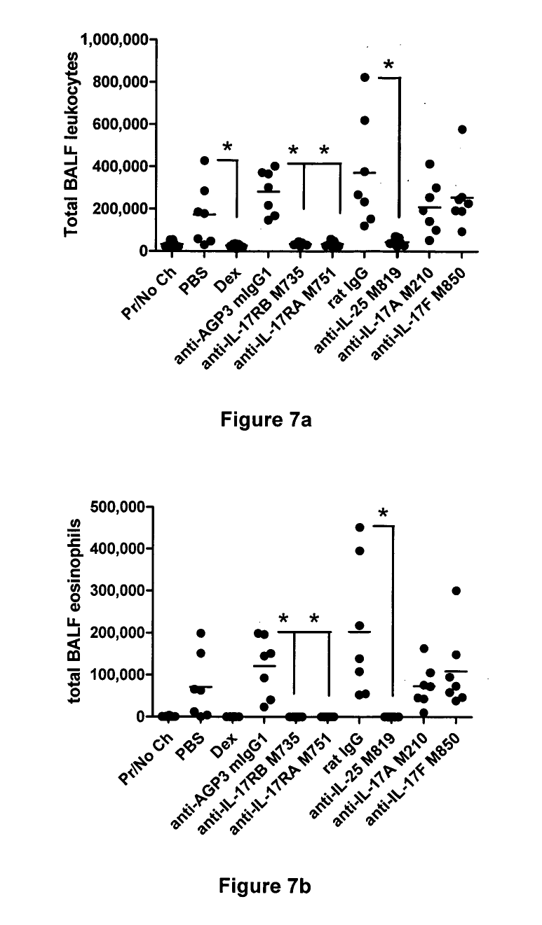

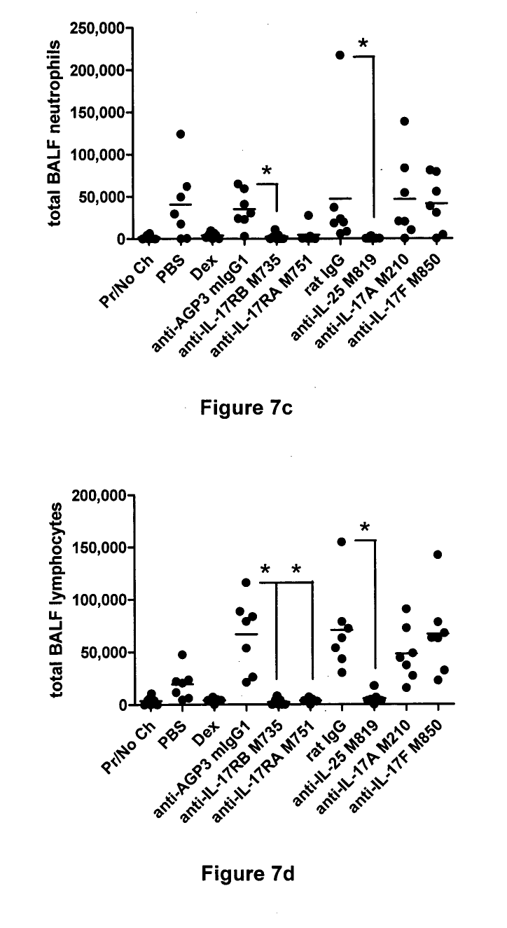

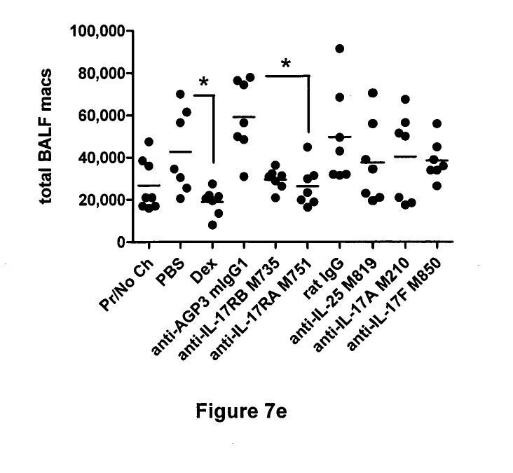

In experiment 3, neutralizing antibodies to IL-17RB, IL-17RA, or IL-25 but not to IL-17A or IL-17F significantly reduced BALF total leukocytes (FIG. 7a), eosinophils (FIG. 7b), and lymphocytes (FIG. 7d) in this mouse OVA asthma model. Neutralizing antibodies to IL-17RB, IL-17RA, or IL-25 but not to IL-17A or IL-17F reduced BALF total neutrophils (FIG. 7c), but only the IL-17RB and IL-25 antibodies had a significant effect. Neutralizing antibodies to IL-17RB or IL-17RA but not to IL-25, IL-17A or IL-17F reduced BALF total macrophages (FIG. 7e), but only the IL-17RA antibody had a significant effect.

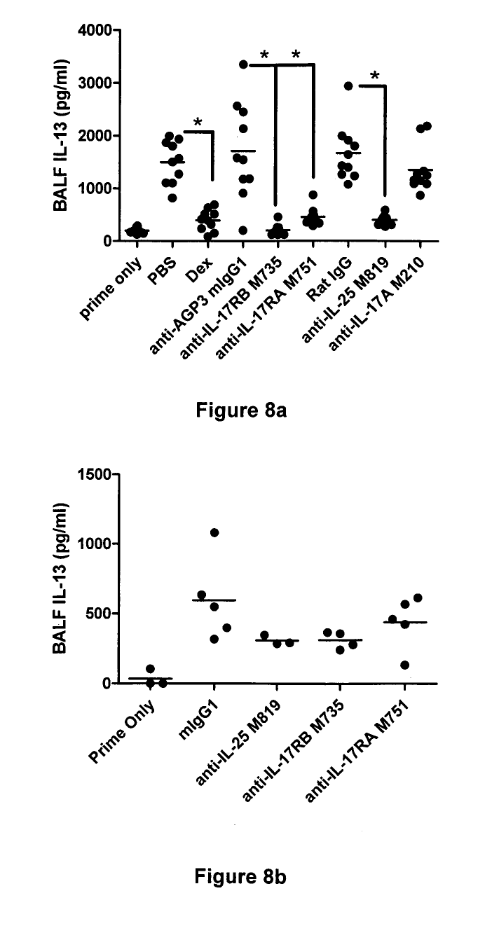

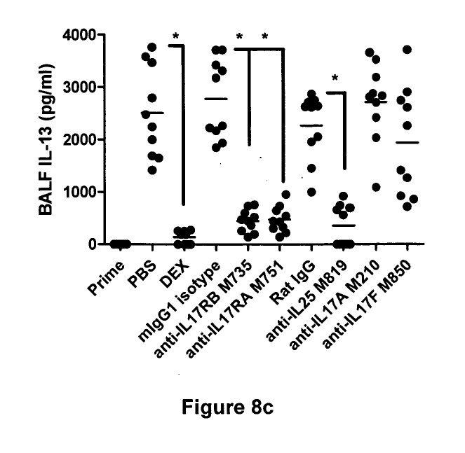

Neutralizing antibodies to IL-17RB, IL-17RA, or IL-25, but not to IL-17A or IL-17F, significantly reduced BALF IL-13 concentrations as shown in FIG. 8a (Experiment 1) and FIG. 8c (Experiment 3). BALF IL-13 concentrations were lower in mice treated with neutralizing antibodies to IL-17RB, IL-17RA, or IL-25 but not significantly in Experiment 2, possibly due to a lower level of IL-13 induction overall compared with that typically observed in this mouse model (FIG. 8b).

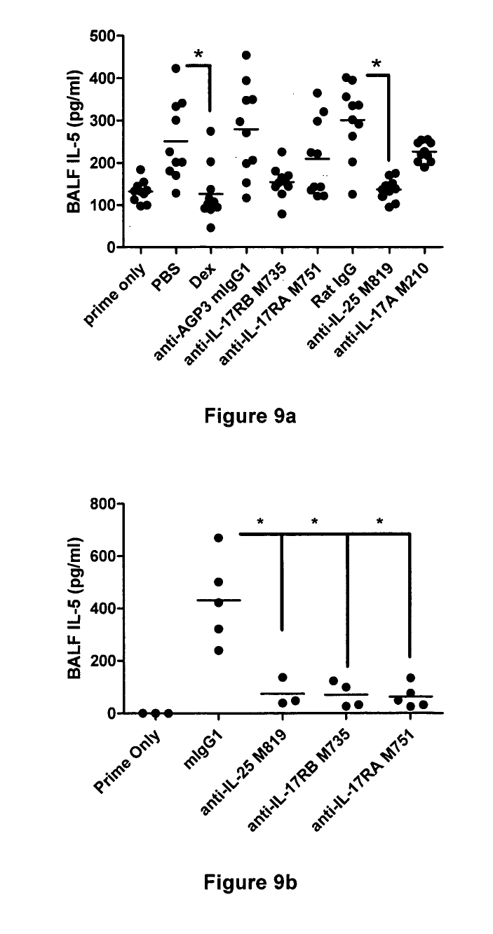

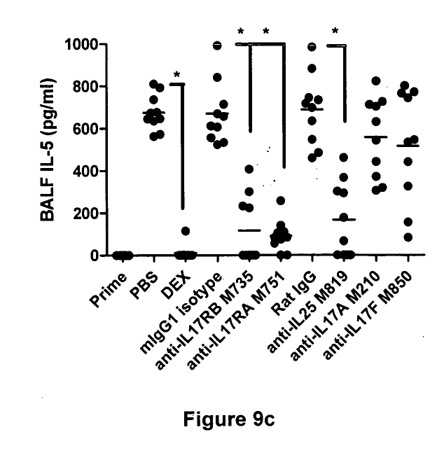

Neutralizing antibodies to IL-17RB, IL-17RA, or IL-25, but not to IL-17A or IL-17F, also reduced BALF IL-5 concentrations in this mouse OVA asthma model, but in Experiment 1 only the anti-IL-25 mAb treated group was significantly lower compared with isotype control antibody treated mice (FIG. 9a), while in Experiment 3 the anti-IL-17RB, anti-IL-17RA, and anti-IL-25 mAb treated groups were all significantly reduced (FIG. 9c). Moreover, BALF IL-5 concentrations were significantly reduced by treatment with neutralizing antibodies to IL-17RB, IL-17RA, and IL-25 in Experiment 2 (FIG. 9b).

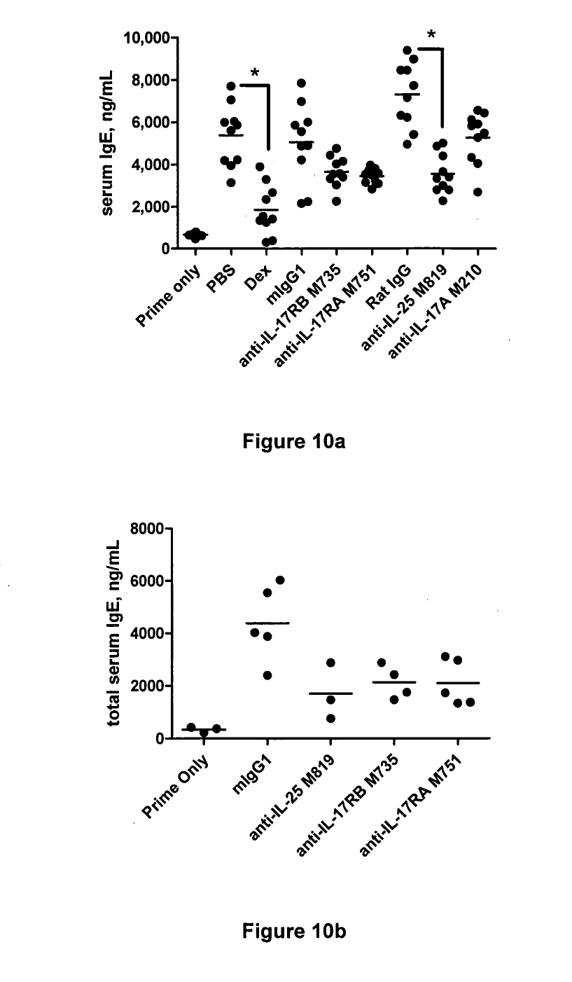

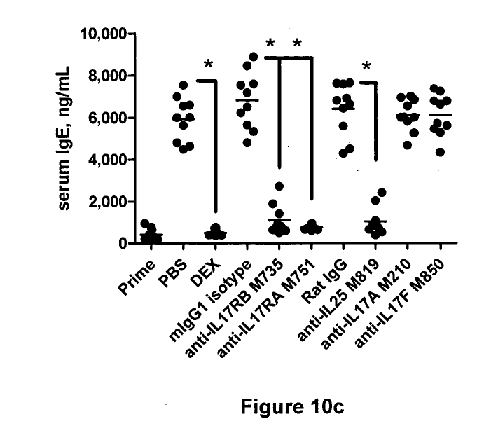

Similarly, neutralizing antibodies to IL-17RB, IL-17RA, or IL-25, but not to IL-17A or IL-17F, reduced total serum IgE concentrations in this mouse OVA asthma model. In Experiment 1, neutralizing antibodies to IL-17RB, IL-17RA, or IL-25 reduced total serum IgE concentrations, but only the group treated with the neutralizing antibody to IL-25 significantly reduced total serum IgE concentrations compared with the appropriate isotype control antibody treated group (FIG. 10a). In Experiment 2, neutralizing antibodies to IL-25, IL-17RB, or IL-17RA reduced total serum IgE concentrations but not significantly compared with the control antibody treated group (FIG. 10b). In Experiment 3, neutralizing antibodies to IL-17RB, IL-17RA, or IL-25 but not to IL-17A or IL-17F significantly reduced total serum IgE concentrations compared with their appropriate control antibody treated groups (FIG. 10c).

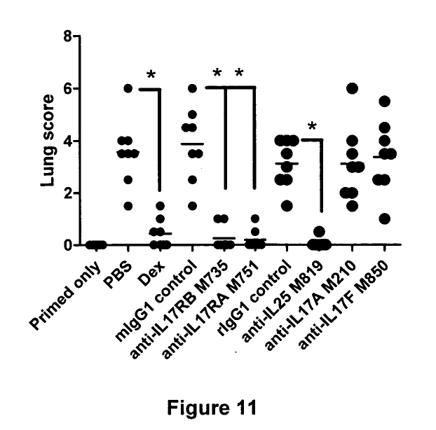

The lungs from 8 mice from each treatment group in Experiment 3 were histologically analyzed. Lung tissue sections were stained with H&E or PAS, and then scored by a pathologist as described previously for Example 1. Treatment with neutralizing antibodies to IL-17RB, IL-17RA, or IL-25 but not to IL-17A or IL-17F significantly reduced inflammation scores in this mouse OVA asthma model (FIG. 11).

These results demonstrated that treatment with anti-IL-17RB mAb M735, anti-IL-17RA mAb M751, or anti-IL-25 mAb M819 significantly decreased multiple parameters of inflammation in this mouse OVA-induced asthma model, which is regarded as a model of pulmonary inflammation conditions such as asthma in humans. In contrast, treatment with anti-IL-17A mAb or anti-IL-17F mAb did not significantly decrease inflammation in this model. Thus, IL-25 and its receptors IL-17RB and IL-17RA play a role in mediating inflammation in this mouse OVA asthma model.

1-15. (canceled)

16. A method of inhibiting IL-17RA-IL-17RB heteromeric receptor complex activation, comprising exposing a cell expressing at least IL-17RA and IL-17RB to an IL-17RA-IL-17RB antagonist such that activation of an IL-17RA-IL-17RB heteromeric receptor complex by IL-25 is partially or fully inhibited.

17. The method of claim 16, wherein the IL-17RA-IL-17RB antagonist is an antigen binding protein.

18. The method of claim 17, wherein the antigen binding protein binds the IL-17RA-IL-17RB heteromeric receptor complex or a subunit thereof.

19. The method of any one of claims 16-18 wherein formation of an IL-17RA-IL-17RB heteromeric receptor complex is partially or fully inhibited.

20. The method of any one of claims 16-18 wherein release of at least one proinflammatory mediator is partially or fully inhibited.

21. The method of claim 20, wherein the proinflammatory mediator is selected from the group consisting of: IL-5, IL-6, IL-8, IL-13, CXCL1, CXCL2, GM-CSF, G-CSF, M-CSF, IL-1β, TNFα, RANK-L, LIF, PGE2, IL-12, MMP3, MMP9, GROα, and NO.

22. A method of inhibiting IL-17RA-IL-17RB heteromeric receptor complex activation in vivo, comprising exposing a cell expressing at least IL-17RA and IL-17RB to an IL-17RA-IL-17RB antagonist such that activation of an IL-17RA-IL-17RB heteromeric receptor complex by IL-25 is partially or fully inhibited.

23. The method of claim 22, wherein the IL-17RA-IL-17RB antagonist is an antigen binding protein.

24. The method of claim 23, wherein the antigen binding protein binds the IL-17RA-IL-17RB heteromeric receptor complex or a subunit thereof.

25. The method of claim 22, wherein formation of an IL-17RA-IL-17RB heteromeric receptor complex is partially or fully inhibited.

26. The method of claim 23, wherein formation of an IL-17RA-IL-17RB heteromeric receptor complex is partially or fully inhibited.

27. The method of claim 24, wherein formation of an IL-17RA-IL-17RB heteromeric receptor complex is partially or fully inhibited.

28. The method of claim 22, wherein release of at least one proinflammatory mediator is partially or fully inhibited.

29. The method of claim 23, wherein release of at least one proinflammatory mediator is partially or fully inhibited.

30. The method of claim 24, wherein release of at least one proinflammatory mediator is partially or fully inhibited.

31. The method of claim 25, wherein release of at least one proinflammatory mediator is partially or fully inhibited.

32. The method of any one of claims 28-31, wherein the proinflammatory mediator is selected from the group consisting of: IL-5, IL-6, IL-8, IL-13, CXCL1, CXCL2, GM-CSF, G-CSF, M-CSF, IL-1β, TNFα, RANK-L, LIF, PGE2, IL-12, MMP3, MMP9, GROα, and NO.

33. The method of claim 22, wherein the IL-17RA-IL-17RB antagonist is administered to an individual afflicted with an autoimmune or inflammatory disease.

34. The method of claim 23, wherein the IL-17RA-IL-17RB antagonist is administered to an individual afflicted with an autoimmune or inflammatory disease.

35. The method of claim 24, wherein the IL-17RA-IL-17RB antagonist is administered to an individual afflicted with an autoimmune or inflammatory disease.

36. The method of claim 25, wherein the IL-17RA-IL-17RB antagonist is administered to an individual afflicted with an autoimmune or inflammatory disease.

37. The method of any one of claims 33-36, wherein the autoimmune or inflammatory disease is selected from the group consisting of Acute Respiratory Disorder Syndrome (ARDS), respiratory distress syndrome, bronchitis, and airway hyperresponsiveness associated with viral-induced conditions such as respiratory syncytial virus (RSV), parainfluenza virus (PIV), rhinovirus (RV) and adenovirus.

38. The method of any one of claims 33-36, wherein the IL-17RA-IL-17RB antagonist partially or fully reduces or ameliorates the signs and/or symptoms of the autoimmune or inflammatory disease.

39. The method of claim 37, wherein the IL-17RA-IL-17RB antagonist partially or fully reduces or ameliorates the signs and/or symptoms of the autoimmune or inflammatory disease.


SEQUENCE LISTING (53 sequences)

SEQ ID NO: 1 - protein, 131 aa, Homo sapiens
QVQLQESGPGLVKPSETLSLTCTVSGGSISNYYWNWIRQSPGKGLEWIGDIYYSGSTNYNPSLKSRVTISVDTSKNQFSLKLSSVTTADTAVYYCARDGELANYYGSGSYQFYYYYGMDVWGQGTTVTVSS

SEQ ID NO: 2 - protein, 127 aa, Homo sapiens
QVQLQQWGAGLLKPSETLSLTCAVSGGSFSGYYWSWIRQPPGKGLEWIGEINHSGRTNYNPSLKSRVTISVDTSKNQFSLKLSSVTAADTAVYYCARGPYYFDSSGYLYYYYGLDVWGQGTTVTVSS

SEQ ID NO: 3 - protein, 114 aa, Homo sapiens
QVQLVESGGGVVQPGRSLRLSCAASGINFSSYGMHWVRQAPGKGLEWVAVIWYDGSNKHYADSVKGRFTISRDNSKNTLYLQMNSLRAEDTAVYYCARDTGVYWGQGTLVTVSS

SEQ ID NO: 4 - protein, 114 aa, Homo sapiens; identical to SEQ ID NO: 3

SEQ ID NO: 5 - protein, 125 aa, Homo sapiens
QVQLQESGPGLVKPSETLPLTCTVSGGSIRSYYWSWIRQPAGKGLEWIGRIYRSGNTIYNPSLKSRVTMSIDTSKNQFSLTLSSVTAADTAVYYCARENYSESSGLYYYYGMDVWGQGTTVTVSS

SEQ ID NO: 6 - protein, 124 aa, Homo sapiens
QVQLVQSGAEVKKPGASVKVSCKASGYTLTRYGISWVRQAPGQGLEWMGWISAYNGNTNYAQKLQGRVTMTTDTSTSTAYMELRSLRSDDTAVYYCARRDYDILTGYYNGFDPWGQGTLVTVSS

SEQ ID NO: 7 - protein, 124 aa, Homo sapiens; identical to SEQ ID NO: 6

SEQ ID NO: 8 - protein, 124 aa, Homo sapiens
QVQLVQSGAEVKKPGASVKVSCKASGNTFTGYGISWVRQAPGQGLEWMGWISAYNGNTNYAQNLQGRVTMTTDTSTSTAYMELRSLRSDDTAVYYCARRDYDILTGYYNGFDPWGQGTLVTVSS

SEQ ID NO: 9 - protein, 124 aa, Homo sapiens
QVQLVQSGVEVKKPGASVKVSCKASGYTLTRYGISWVRQAPGQGLEWMGWISAYNGNTNYAQKLQGRVTMTTDTSTSTAYMELRSLRSDDTAVYYCARRDYDILTGYYNGFDPWGQGTLVTVSS

SEQ ID NO: 10 - protein, 124 aa, Homo sapiens
QVQLQESGPGLVKPSQTLSLTCTVSGGSISSGGYYWSWIRQHPGKGLEWIGYIYFSGSAYYNPSLKSRVAISVDTSKNQFSLKLSSVTAADTAVYYCAREYYDSSGYPDAFDIWGQGTMVTVSS

SEQ ID NO: 11 - protein, 114 aa, Homo sapiens
QVQLVESGGGVVQPGRSLRLSCATSGITFSSYGMHWVRQAPGKGLEWVAVIWYDGSNKYYADSVKGRFTISRDNSKNTLYLQMNSLRAEDTAVYYCARDTKDYWGQGTLVTVSS

SEQ ID NO: 12 - protein, 116 aa, Homo sapiens
QVQLVQSGAEVKKPGASVKVSCKASGYTLTSYGISWVRQAPGQGLEWMGWISTYKGNTNYAQKLQGRVTMTTDTSTSTAYMELRSLRSDDTAVYYCARKQLVFDYWGQGTLVTVSS

SEQ ID NO: 13 - protein, 121 aa, Homo sapiens
QVQLVESGGGVVQPGRSLRLSCAASGFTFSSYGMQWVRQAPGKGLEWVAVIWYDGNKKYYADSVKGRFTISRDNSKNTLYLQMNSLRAEDTAVYYCARGRVRDYYYGMDVWGQGTTVTVSS

SEQ ID NO: 14 - protein, 116 aa, Homo sapiens
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 15 - protein, 121 aa, Homo sapiens; identical to SEQ ID NO: 13

SEQ ID NO: 16 - protein, 116 aa, Homo sapiens
QVQLVQSGAEVKKPGASVKVSCKASGYTFTSYGISWVRQAPGQGLEWMGWISAYNGNTKYAQKLQGRVTMTTDTSTSTVYMELRSLRSDDTAVYYCARKQLVFDYWGQGTLVTVSS

SEQ ID NO: 17 - protein, 116 aa, Homo sapiens
QVQLVQSGAEVKKPGAAVKVSCKATGYTLTSYGISWVRQAPGQGLEWMGWISAYSGNTKYAQKLQGRVTMTTDTSTSTAYMELRSLRSDDTAVYYCARKQLVFDYWGQGTLVTVSS

SEQ ID NO: 18 - protein, 126 aa, Homo sapiens
QVQLVQSGAEVKKPGASVKVSCKASGYSFTDYYMHWVRQAPGQGLEWMGWMHPNSGGTDLAQRFQGRVTMTRDTSISTAYMELSRLRSDDTAVYYCARGGYCSTLSCSFYWYFDLWGRGTLVTVSS

SEQ ID NO: 19 - protein, 116 aa, Homo sapiens
QVQLVQSGAEVKKPGASVKVSCKASGYTLTSYGISWVRQAPGQGLEWMGWISAYSGNTKYAQKFQGRVTMTTDTSTSTAYMELRSLRSDDTAVYYCARRQLALDYWGQGTLVTVSS

SEQ ID NO: 20 - protein, 118 aa, Homo sapiens
EVQLVESGGGLVQPGGSLRLSCAASGFTFSSYSMNWVRQAPGKGLEWVSFISARSSTIYYADSVKGRFTISRDNAKNSLYLQMNSLRDEDTAVYYCARPKVGGGMDVWGQGTTVTVSS

SEQ ID NO: 21 - protein, 118 aa, Homo sapiens
EVQLVESGGGSVQPGGSLRLSCAASGFTFSSYSMNWVRQAPGKGLEWVSIISSRSSIIHYADSVKGRFTISRDNAKNSLYLQMNSLRDEDTAVYYCARPKVGGGMDVWGQGTTVTVSS

SEQ ID NO: 22 - protein, 116 aa, Homo sapiens
QVQLVQSGAEVKKPGASVKVSCKASGYTFTRYGISWVRQAPGQGLEWMGWISAYSGNTNYAQKLQGRVTMTTDTSTSTAYMELRSLRSDDTAVYYCARRQLYFDYWGQGTLVTVSS

SEQ ID NO: 23 - protein, 125 aa, Homo sapiens
QVQLQESGPGLVKPSETLSLTCTVSGGSISSYYWSWIRQPAGKRLEWIGRIYPSGRTNYNPSLKSRVTMSVDTSKNQFSLKLSSVTAADTAVYYCAREAYELQLGLYYYYGMDVWGQGTPVTVSS

SEQ ID NO: 24 - protein, 125 aa, Homo sapiens
QVQLQESGPGLVKPSETLSLTCTVSGGSISSYYWSWIRQAAGKRLEWIGRIYPSGRTNYNPSLKSRVTMSVDTSKNQFSLKLSSVTAADTAVYYCAREAYELQLGLYYYYGMDVWGQGTPVTVSS

SEQ ID NO: 25 - protein, 124 aa, Homo sapiens
QVQLQESGPGLVKPSQTLSLTCTVSGGSISSGGYYWSWIRQHPGKGLEWIGYIYYSGNTYYNPSLRSRVTISVDTSKNQFSLKLNSVTAADTAVYYCAREAGGNSAYYYGMDVWGQGTTVTVSS

SEQ ID NO: 26 - protein, 125 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDRTYYFGSGSYEGMDVWGQGTTVTVSS

SEQ ID NO: 27 - protein, 107 aa, Homo sapiens
DILMTQSPSSLSASVGDRVTITCRASQGIRNDLGWYQQKPGKAPKRLIYAASSLQSGVPSRFSGSGSGTEFTLTISSLQPEDFATYYCLQHNSNPFTFGPGTKVDIK

SEQ ID NO: 28 - protein, 106 aa, Homo sapiens
EIVMTQSPATLSVSPGERATLSCRASQSVSRNLVWYQQRPGQAPRLLIYGASTRANGIPARFSGSGSGTEFTLTISSLQSEDFAVYYCQQYKSWRTFGQGSKVEIK

SEQ ID NO: 29 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHKSYPLTFGGGTKVEIK

SEQ ID NO: 30 - protein, 108 aa, Homo sapiens
EIVMTQSPATLSVSPGERATLSCRASQSVSRNLAWYQQKPGQAPRLLIYGASTRATGIPARFSGSGSGTEFTLTISSLQSEDFAVYYCQQYNNWPTWTFGQGTKVEIK

SEQ ID NO: 31 - protein, 107 aa, Homo sapiens
DIQMTQSPSSLSASVGDRVTITCRASQGIRNDLGWYQQKPGKAPKRLIYAASSFQSGVPSRFSGSGSGTGFTLTISSLQPEDFATYYCLQHNSYPPTFGQGTKVEIK

SEQ ID NO: 32 - protein, 107 aa, Homo sapiens; identical to SEQ ID NO: 29

SEQ ID NO: 33 - protein, 107 aa, Homo sapiens; identical to SEQ ID NO: 29

SEQ ID NO: 34 - protein, 107 aa, Homo sapiens; identical to SEQ ID NO: 29

SEQ ID NO: 35 - protein, 107 aa, Homo sapiens; identical to SEQ ID NO: 29

SEQ ID NO: 36 - protein, 107 aa, Homo sapiens
DIQMTQSPSSVSASVGDRVTITCRASQGIRSWLAWYQQKPGKAPKLLIFAASSLQSGVPSRFSGSGSGTEFTLTISSLQPEDFATYYCQQANNFPRTFGQGTKVEIK

SEQ ID NO: 37 - protein, 108 aa, Homo sapiens
EIVMTQSPATLSVSPGERATLSCRASQSVSSNLAWYQQKPGQAPRLLIYGASTRAAGIPARFSGGGSGTAFTLTISNLQSEDFAVYYCQHYINWPKWTFGQGTKVDIK

SEQ ID NO: 38 - protein, 107 aa, Homo sapiens
EIVMTQSPATLSVSPGERATLSCRASQSISSSLAWYQQKPGQAPRLLIYGASTRATGIPARFSGSGSGTEFTLTISSLQSENFAVYYCQQYDNWPLTFGGGTKVEIK

SEQ ID NO: 39 - protein, 112 aa, Homo sapiens
DIVMTQTPLSLSVTPGQPASIACKSSQSLLHSDGKTYLYWYLQKPGQPPQLLIYEVSTRFSGVPDRFSGSGSGTDFTLKISRVEAEDVGVFYCMQSIQLPLTFGGGTKVEIK

SEQ ID NO: 40 - protein, 107 aa, Homo sapiens
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

SEQ ID NO: 41 - protein, 107 aa, Homo sapiens
EIVMTQSPATLSVSPGERVTLSCRASQSVSSNLAWFQQKPGQAPRPLIYDASTRAAGIPARFSGSGSGTDFTLTISSLQSEDFAVYYCQQYDNWPLTFGGGTKVEIK

SEQ ID NO: 42 - protein, 107 aa, Homo sapiens
EIVMTQSPATLSVSPGERATLSCRASQSISTSLAWYQQKPGQAPRLLIYGTSTRATGIPARFSGSGSGTEFTLTISSLQSEDFAVYFCQQYDIWPLTFGGGTKVEIK

SEQ ID NO: 43 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYSCQQYDNWPLTFGGGTKVEIK

SEQ ID NO: 44 - protein, 113 aa, Homo sapiens
DIVMTQSPDSLAVSLGERATINCKTSQSVLYSSKNKNFLAWYQQKPGQPLNLLIYWASTRESGVPDRFSGSGSGTDFTLTISSLQAEDVAVYYCQQYYSTPFTFGPGTKVDIK

SEQ ID NO: 45 - protein, 107 aa, Homo sapiens
EIVMTQSPATLSVSPGERATLSCRASQSISSNLAWYQQKPGQAPRLLIYGASTRATGIPARFSDNGSGTEFTLTISSLQSEDFAVYFCQQYDTWPLTFGGGTKVEIK

SEQ ID NO: 46 - protein, 107 aa, Homo sapiens
DIQMTQSPSSLSASVGDRVTITCRASQGISNYLAWYQQKPGKFPELLIYAASTLQSGVPSRFSGSGSGTDFTLTISSLQPEDVATYYCQKYNRAPFTFGPGTKVDIK

SEQ ID NO: 47 - protein, 107 aa, Homo sapiens; identical to SEQ ID NO: 46

SEQ ID NO: 48 - protein, 107 aa, Homo sapiens; identical to SEQ ID NO: 41

SEQ ID NO: 49 - protein, 107 aa, Homo sapiens
DIQMTQSPSSLSASVGDRVTISCRASQGIINDLGWYQQKPGKAPKRLIYAASSLQSGVPSRFSGSGSGTEFTFTISSLQPEDFATYYCLQHNSYPPTFGQGTKVEIK

SEQ ID NO: 50 - protein, 113 aa, Homo sapiens
DIVMTQSPLSLPVTLGQPASISCRSSQSLVYSDGHTCLNWFQQRPGQSPRRLIYKVSNWDSGVPDRFSGSGSGTDFTLKISRVEADDVGVYYCMQGTHWPLCSFGQGTKLEIK

SEQ ID NO: 51 - protein, 113 aa, Homo sapiens; identical to SEQ ID NO: 50

SEQ ID NO: 52 - protein, 107 aa, Homo sapiens
DIQMTQSPSSLSASVGDRVTITCRASQAISIYLAWFQQKPGKAPKSLIYAASSLQSGVPSKFSGSVSGTDFTLTISSLQPEDFATYYCQQYSSYPRTFGQGTKVEIK

SEQ ID NO: 53 - protein, 107 aa, Homo sapiens
EILMTQSPATLSVSPGERATLSCRASQSVYSNLAWYQQKPGQAPRLLISGASTRATGIPARFSGSGSGTEFTLTISSLQSEDFAVYYCQQYYNWPWTFGQGTKVEIK